(19) United States
(12) Patent Application Publication
(10) Pub. No.: US 2023/0114119 A1
(43) Pub. Date: Apr. 13, 2023

(54) COMPOSITIONS AND METHODS COMPRISING IMPROVED GUIDE RNAs

(71) Applicant: Cornell University, Ithaca, NY (US)

(72) Inventor: Joseph E. PETERS, Ithaca, NY (US)

(21) Appl. No.: 17/906,134

(22) PCT Filed: Mar. 16, 2021
(86) PCT No.: PCT/US2021/022582

§ 371 Date: Sep. 12, 2022

Related U.S. Application Data
(60) Provisional application No. 63/047,209, filed on Jul. 1, 2020.
(60) Provisional application No. 62/990,111, filed on Mar. 16, 2020.

(51) Int. Cl. C12N 15/113; C12N 15/10

(52) U.S. Cl. CPC C12N 15/113; C12N 15/1089; C12N 2310/20

(57) ABSTRACT

Provided are compositions, methods, systems, and kits, for use in CRISPR-based DNA editing. The compositions include RNA polynucleotides that include one or more atypical repeats, and can include truncated spacers. The RNA polynucleotides are used with proteins in systems that include CRISPR and transposon genes, or proteins encoded by the genes. The genes include transposon genes tnsA, tnsB, tnsC, and tniQ, and Cas genes cas8f, cas5f, cas7f, and cas6f. Use of the RNA polynucleotides as guide RNAs that include atypical repeats with the transposon and CRISPR proteins exhibit enhanced transposition, relative to guide RNAs that do not include atypical repeats. Enhanced transposition is demonstrated using representative IF-3b systems.

### CROSS-REFERENCE TO RELATED APPLICATIONS

This application claims priority to U.S. provisional application No. 62/990,111, filed Mar. 16, 2020, and U.S. provisional application No. 63/047,209, filed Jul. 1, 2020, the entire disclosures of each of which are incorporated herein by reference.

### STATEMENT REGARDING FEDERALLY FUNDED RESEARCH OR DEVELOPMENT

This invention was made with government support under grant number R01GM129118 awarded by National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Mar. 12, 2021, is named 018617_01284_SL.txt and is 2,176,959 bytes in size.

### FIELD

The present disclosure relates generally to approaches for modifying DNA, and more particularly, to improved compositions and methods for CRISPR-based editing, using guide RNAs with sequences that include atypical repeat sequences. The guide RNAs may also or alternatively comprise and shortened spacers.

### BACKGROUND

A wide variety of clustered regularly interspaced short palindromic repeats (CRISPR) arrays have been investigated over the past several years. CRISPR arrays typically comprise an AT-rich leader sequence followed by short repeats that are separated by spacers which each comprise distinct sequences. CRISPR repeats typically span lengths of 28 to 37 base pairs, although shorter and longer sequences have been reported.

RNA polynucleotides transcribed from CRISPR arrays are processed by a variety of mechanisms in order to facilitate RNA-guided editing of polynucleotides using so-called guide RNAs, commonly referred to as gRNAs. The adoption of numerous CRISPR-based RNA guided DNA editing systems and techniques has recently proliferated dramatically, in accordance with increased knowledge and resources that permit rational design of guide RNAs for targeting virtually and DNA sequence in any cell type. However, there remains an ongoing and unmet need for improved compositions and methods to enhance these approaches. The present disclosure is pertinent to this need.

### SUMMARY OF THE DISCLOSURE

The present disclosure provides compositions, methods, systems, and kits, for use in CRISPR-based DNA editing. The disclosure demonstrates that certain CRISPR systems which use privatized guide RNAs exhibit enhanced transposition efficiency. The enhanced transposition efficiency supports use of the described systems to insert cargo DNA at a predetermined location in a DNA substrate, such as a chromosome or plasmid.

The privatized guide RNAs comprise one or more atypical repeat sequences, as further described herein, and may also include truncated spacers. The atypical repeats are, in certain embodiments, derived from one or more repeats that are next to a spacer in a CRISPR array that was not the most recently acquired spacer in the array.

In an embodiment, the disclosure provides an RNA polynucleotide (e.g., a guide RNA, also referred to as a “gRNA”) for use in a CRISPR system that in certain examples is a Type I-F3b CRISPR system.

In embodiments, the RNA polynucleotide comprise contiguously in a 5′ to 3′ direction: i) a 5′ end segment comprising a first CRISPR repeat sequence; ii) a spacer sequence that comprises a targeting sequence that is complementary to a protospacer (e.g., a target sequence) in a DNA target; and iii) a 3′ end segment comprising a second CRISPR repeat sequence. The 5′ end segment, the 3′ end segment, or both, comprise one or more nucleotide changes relative to a first reference repeat sequence, or a second reference repeat sequence, respectively, or a combination of such nucleotide changes. In embodiments, the RNA polynucleotide is functional with a Type IF-3b CRISPR system and exhibits more efficient modification of DNA templates comprising the protospacer than an RNA polynucleotide used as a guide RNA in the Type IF-3b CRISPR system, but wherein the guide RNA does not comprise the one or more nucleotide changes, e.g., the guide RNA does not contain an atypical repeat. In embodiments, the guide RNA includes a 5′ end segment that comprises or consists of 8 nucleotides. In embodiments, the guide RNA includes a 3′ end segment that comprises or consists of 20 nucleotides, and optionally, the 3′ end of the 20 nucleotides is a G. In embodiments, the 3′ end segment of the guide RNA forms a stem loop that comprises palindromic sequences.

In non-limiting examples, an RNA polynucleotide of the disclosure includes, as a reference sequence, a first repeat reference sequence that is encoded by a first occurring repeat sequence that is 3′ to a Cas6 coding sequence in an endogenous prokaryotic CRISPR array. In embodiments, a second reference repeat sequence is encoded by a second occurring repeat sequence that is 3′ to the Cas6 coding sequence in the endogenous prokaryotic CRISPR array. In embodiments, the first and/or second reference repeat sequence is the same as a repeat sequence present in a bacterium or archaea, wherein the repeat sequence in the bacterium or archaea is contiguous with a spacer in a CRISPR array that is not the most recently acquired spacer acquired by the bacterium, e.g., the 3′ end of the first repeat is next to the 5′ end of a spacer that was not the most recent spacer that was inserted into the array. Likewise, the 3′ end of the spacer is next to the 5′ nucleotide of the second repeat in the described repeat-spacer-repeat segment. In embodiments, the endogenous prokaryotic CRISPR array is may be a gammaproteobacteria CRISPR array. In a non-limiting embodiment, the reference repeats, and/or the atypical repeats, may be obtained from an A. salmonicida CRISPR array.

The disclosure includes the described RNA polynucleotides that are provided as a component of a ribonucleoprotein (RNP) complex. In embodiments, the RNP comprises a described guide RNA and proteins that are selected Cas5, Cas6, Cas7, Cas8, and combinations thereof. In an embodiment, the RNP comprises the Cas6, and a stem loop comprising at least a portion of the 3′ end segment of an atypical repeat is recognized by the Cas6 in the RNP. In embodiments, the targeting sequence of the guide RNA is selected for inclusion in the RNA polynucleotide that is processed into a guide RNA, such that the RNA polynucleotide is suitable for use in CRISPR-based modification of a known DNA target sequence comprising a protospacer. In embodiments, the targeting sequence (e.g., the spacer) in the guide RNA may be completely identical to the protospacer, or certain mismatches between the spacer and the protospacer may be included. In certain embodiments, the spacer is not more than 29 nucleotides in length, and thus may constitute a truncated spacer. In embodiments, an RNA polynucleotide of the disclosure comprises only one repeat-spacer-sequence, or more than one repeat-spacer-repeat sequence, wherein at least one of the repeat sequences is an atypical repeat. In embodiments, the spacer in a described repeat-spacer-repeat sequence may, where there is more than one repeat-spacer-repeat segment in the RNA polynucleotide, be the same spacer sequence, or different spacer sequences may be used.

The disclosure includes expression vectors encoding all of the RNA polynucleotides described herein, including but not limited to all atypical repeats, and all combinations of atypical repeats. Isolated RNA polynucleotide transcribed from such an expression vector are also included, as are cells, including eukaryotic and prokaryotic cells, that include the expression vectors.

In one aspect, the disclosure provides a system for modifying a genetic target in one or more cells. The system includes the described RNA polynucleotides, or one or more vectors encoding them, and also includes a first set of transposon genes tnsA, tnsB, tnsC, and tniQ, Cas genes cas8f, cas5f cas7f and cas6f and optionally an xre gene encoding a transcription regulator, or optionally one or more proteins encoded by one or more of said genes. In embodiments, at least two of the described proteins may be present in a fusion protein.

The system also includes a DNA cargo that can be introduced into DNA in a location that is proximal to the protospacer in a DNA target. In non-limiting embodiments, genes, or proteins encoded by the genes, that are used in the described systems, optionally comprise one or more amino acid changes, relative to a reference sequence. In embodiments, the amino acid changes can be in the tnsA gene, the tnsB gene, the tnsC gene, or other genes and proteins described herein as components of the system.

In an aspect, the disclosure includes a method that comprises introducing or expressing a described system in cells. In embodiments, the methods are suitable for modifying prokaryotic or eukaryotic cells. In embodiments, the targeting sequence in the RNA polynucleotide that comprises a described guide RNA is targeted to a protospacer in a chromosome or a plasmid in the cells. The described method includes introducing a cargo DNA into the cells. The cargo DNA is inserted into the chromosome or plasmid in a position that is proximal to the protospacer. In embodiments, the DNA cargo is inserted into the chromosome or the plasmid at a position that is 48 nucleotides from an end of the protospacer. In certain embodiments, the DNA cargo comprises transposon left and right ends.

In another aspect, the disclosure provides a method comprising analyzing CRISPR arrays from a plurality of organisms, determining repeat sequences flanking spacers in the CRISPR arrays, comparing repeat sequences flanking earlier acquired spacers to repeat sequences flanking later acquired spacers, determining differences between repeat sequences flanking the earlier and later acquired spacers, and designating the repeat sequences flanking the earlier acquired spacers that are different from the repeat sequences flanking the later acquired spacers as candidates for use in designing a guide RNA for use in CRISPR-based DNA modification. In embodiments, the disclosure includes producing an RNA polynucleotide that includes sequences identified using the described method. The disclosure further comprises using the RNA polynucleotides identified using the described method in CRISPR-based DNA modifications, which may include insertion of a cargo DNA into a chromosome or plasmid. Thus, in embodiments, the disclosure includes providing, and using, RNA polynucleotides that contain a substitution of a spacer or a repeat, or a combination thereof, in analyzed CRISPR arrays with a different spacer and/or repeat sequence. In embodiments, the spacer is optionally not longer than 29 nucleotides in length. The disclosure includes libraries comprising RNA polynucleotides identified and produced according to the described method, wherein the RNA polynucleotides include a spacer that is targeted to a segment of DNA. The spacer sequence may be designed by a user of the system.

The disclosure also include a database comprising a plurality of entries comprising sequences identified by the described method. The disclosure further comprises selecting a sequence from the described database, and producing an expression vector and/or an RNA polynucleotide that comprises an identified sequence.

In another aspect, the disclosure includes a kit for producing an expression vector for use in CRISPR-based DNA modification. The includes an expression vector comprising one or more restriction endonuclease recognition sites configured for cloning a desired spacer such that the spacer is contiguous with one or more repeat sequences identified according to the method of claim, or other atypical repeat sequences as described further herein. The kit may also include expression vectors that comprise some or all of tnsA, tnsB, tnsC, and tniQ genes, Cas genes cas8f, cas5f cas7f, and cas6f and optionally an xre gene, or one or more proteins encoded by one or more of these genes.

### BRIEF DESCRIPTION OF THE FIGURES

FIG. 1. Tn7-like elements with I—F3 CRISPR-Cas systems found in gamma proteobacteria. A TnsA protein similarity tree representing 802 elements indicated by host strain. Elements that were >90 percent identical are indicated with a single representative. A similarity score was calculated for repeats and indicated in shades of black (high) to grey (low). Spacers are indicated with rectangles (shorter rectangles indicate truncated spacers).

FIG. 2. Selected representatives from four att-site families of Tn7-like elements with I—F3 CRISPR-Cas systems. Representatives for three major families (att sites; yciA, guaC, andffs) and one minor family (rsmJ) are indicated by host. (A) Transposition genes (tnsA, tnsB, tnsC, and tniQ/tnsD), Cas genes cas6, cas7, and cas8/5, and regulator xre are indicated. CRISPR arrays indicated as in FIG. 1. The left (L) and Right (R) ends of the elements are indicated and putative Xre binding sites (asterisks). (B) Matches between the guide RNA and protospacer are shown on each gene (block arrow) with the right end of the element (grey box) and host indicated. Distance from the protospacer to the target site duplication (TSD, grey and bracketed) is shown. FIG. 2B discloses SEQ ID NOS: 5731-5741, respectively, in order of appearance. (C) CRISPR array is indicated with the leader region and spacer (S #) and repeats (R #) indicated showing the sequence of the repeats. Sequence differences from the first repeat (red), noting changes maintaining the stem (light blue) and the inverted repeats that makes the stem (boxed) are indicated. The size of the gap in the array is indicated noting the putative Xre regulatory site. FIG. 2C discloses SEQ ID NOS: 4762, 4762-4766, 374-376, 3021, 3021-3024 and 1286-1289, respectively, in order of columns.

FIG. 3. I-F3b Tn6900 element derived from A. salmonicida S44 allows RNA-guided transposition with typical and atypical repeats. Various transposition targets were tested using the A. salmonicida S44 native array or as individual repeat-spacer-repeat units. (A) Simplified representation of transposition/CRISPR-associated genes, CRISPR array (marked as in FIG. 1) and the resulting typical and atypical guide RNAs with the 5′ and 3′ handles indicated. Position of Cas6 processing is indicated (scissors). FIG. 5A discloses SEQ ID NOS: 5742-5744, respectively, in order of appearance. (B) Frequency of transposition found with the native A. salmonicida S44 array with targets constructed into an F plasmid; A. salmonicida S44 plasmid pS44-1 (pS44-1), chromosomal ffs att site (ffs) or a negative control, lacZ gene. (lacZ) (C-E) Transposition frequency found with a single repeat-spacer-repeat unit in various combinations of spacers with typical or atypical repeats from Tn6900 with the indicated targets constructed into an F plasmid. All data indicate mean+/−standard deviation (n=3).

FIG. 4. Analysis of atypical repeat sequences in Tn7-CRISPR-Cas elements, Tn6677 and Tn6900. (A) Consensus sequence of the typical and atypical repeats as a function of position in the array. Symbols are as in previous figures with the stem-loop indicated (top, n=85 for I-F3a, n=74 for I-F3b), (middle, n=51 for I-F3a, n=41 for I-F3b) or (bottom, n=51 for I-F3a, n=41 for I-F3b). (B-D) Frequency of transposition found with changes in typical and atypical guide RNAs for Tn6900 (B) or Tn6677 (C and D) with the indicated spacers and their associated targets. FIG. 4B discloses SEQ ID NOS: 5743, 5745-5746 and 5744, respectively, in order of appearance. FIG. 4D discloses SEQ ID NOS: 5747-5750, respectively, in order of appearance. Typical guide RNA were tested, comparing with naturally-occurring changes from atypical repeats (red) or engineered mutations (underlined). All data indicate mean+/−standard deviation (n=3).

FIG. 5. P. aeruginosa type I-F1 Cascade can utilize heterologous I—F3 CRISPR arrays in a plasmid interference assay, but mismatches and I-F3b atypical guide RNAs allow privatization. Expression of P. aeruginosa Cas proteins with various arrays reduces transformation efficiency for protospacer containing plasmid, but not control. Single unit arrays from P. aeruginosa PA14 and A. salmonicida S44 Tn6900 with ffs spacer, and V. cholerae HE-45 Tn6677 with guaC spacer were tested in typical and atypical repeat configurations as indicated. Spacers were either perfectly matched to protospacer or contained native mismatches. Repeat configuration sequences are presented in FIG. 10B. All data indicate mean+/−standard deviation (n=3).

FIG. 6. Xre proteins regulate components of RNA-guided transposition. (A-B) Consensus sequence for putative Xre binding motifs in I-F3a and I-Fb elements. (C-D) Xre-binding resolved by EMSA. DNA fragments with the transcription control regions were incubated with increasing amounts of Xre protein from the respective element before electrophoresis (100 nM DNA; protein:DNA ratios=0, 2, 5, 10, 20:1). (E-F) Promoter function resolved by LacZ expression monitored by Miller units at various arabinose controlled Xre expression levels. Transcription control regions are indicated from V. cholerae HE-45 Tn6677 (Vc), V. parahaemolyticus RIMD221063 (Vp). A. salmonicida S44 Tn6900 (As), and Vibrio sp. 10N.286.45.B6 (VB6).

FIG. 7. Xre regulation program allows zygotic induction of transposition function promoters in a new host after conjugal transfer. Transfer of lacZ fused to Xre transcriptional control regions results in a burst of expression in a naïve recipient strain, but not recipients expressing the Xre regulator protein. Donor and recipient were plated together for mating (solid lines) or plated separately (dashed lines) as a control. Cells were harvested and controls mixed and LacZ expression monitored by Miller units as indicated. All data indicate mean+/−standard deviation (n=3).

FIG. 8. Aeromonas element features and transposition. (a) Schematic representation of two nearly-identical I-F3b Tn7-CRISPR-Cas elements found in different bacterial species suggesting recent activity. Core features are indicated as in FIG. 2a. Elements are located either in the chromosomal ffs site in A. hydrophila AFG_SD03 or inserted into a phosphoadenosine phosphosulfate reductase gene (cysH) found on a large conjugal plasmid (pS44-1) in A. salmonicida S44. The A. hydrophila element is split across several contigs interrupted by apparent IS element insertions. (b) Spacers in the leader-proximal position of A. salmonicida S44 and A. hydrophila AFG_SD03 CRISPR arrays match protospacers in a plasmid encoded cysH. Relative position of the protospacers are indicated. Distance from the edge of the protospacer matching A. salmonicida S44 spacer to the central position in the target site duplication (TSD), the 5 bp TSD (underlined), as well as the terminal sequence of the transposon ends are shown. FIG. 8B discloses SEQ ID NOS: 5751-5752, respectively, in order of appearance. (c) Repeats and spacers from Tn7-like CRISPR arrays in A. salmonicida S44 and A. hydrophila AFG_SD03. Repeats are annotated as in FIG. 2c. Differences from the first repeat are indicated in red. Matches between the guide RNA and protospacer are indicated by a short vertical line. The putative I-F PAM is underlined. FIG. 8C discloses SEQ ID NOS: 5802-5803, 5753, 5754, 5804, 5755-5758, 777-781, 5759, 5760, 5757, and 5805, respectively, in order of columns. (d) Protospacers on the chromosome or F plasmid are targeted at high efficiency with atypical guide RNA complexes. The same three lacZ guide RNA complexes were tested with either the F::/acZ plasmid or chromosome (lacZ in its native position) and insertion events were indicated by generating white versus red colonies on MacConkey's lactose indicator media. Graph shows the mean+/−standard deviation of three biological replicates and number of white colonies and total colonies observed. (e) Different genes on the chromosome can be targeted for atypical guide RNA-directed transposition in the E. coli chromosome. Two genes where tested with two spacers each (top and bottom strand) for galactose (galK) and sorbitol (srlD). Transposition frequency was assayed by monitoring gene inactivation leading to loss of sugar catabolism as visualized by white versus red colonies on the appropriate MacConkey's indicator media. Graph shows the mean+/−standard deviation of three biological replicates.

FIG. 9. Assaying full transposition frequency and position. (a) Mate out assay schematic. Target DNAs with the appropriate protospacer are recombined onto an F plasmid and transposition genes and arrays are supplied on expression vectors to mobilize a mini-Tn donor element located in the chromosome (Methods). After induction, transposition frequency is determined by mating out the population of F plasmids into a donor strain and quantifying antibiotic marker presence in transconjugants as shown. (b) Transposition position and orientation in transconjugants are determined by PCR. An internal primer and two primers flanking the target site capture orientation of insertion. For Tn6900, pS44-1-targeted insertions were monitored; for Tn6677, guaC^{Vc}-targeted insertions were monitored. *For Tn6900 typical array insertion isolates 12, 13, and 14, the first PCR reaction failed and was repeated with the same template strain. (c) Transposition position and target site duplication are confirmed by Sanger sequencing for Tn6900 transposition. Arrows indicate position of the central base of the target site duplication for isolated transposition events targeting pS44-1, with distance from protospacer to the central position of the target site duplication listed for eight transposition events, confirming previously described target site wobble. Graph shows one representative of the actual target site duplication (TSD). FIG. 9C discloses SEQ ID NOS: 5759, 5761-5765, and 5763, respectively, in order of appearance.

FIG. 10. Interference assay repeat sequences. Repeat sequences used in interference assays (SEQ ID NOS: 5766-5770, respectively, in order of appearance). Differences from P. aeruginosa repeats are indicated in grey. The box indicates the previously established conserved region that comprises the putative stem-loop in I-F repeats. N32 indicates the position encoded in the spacer.

FIG. 11. I—F3 Xre cluster into two clades with restriction-modification C proteins. (a) Similarity tree of Xre (midpoint rooted) with associated C proteins C.AhdI and C.Csp231I (marked in teal and fuchsia), indicating clustering in the two branches. Features are indicated as in FIG. 1. (b) Predicted regulator sequences for Xre and associated C proteins. Conserved inverted motif sequences are indicated by bold text and black arrows. The start codon of the downstream gene is underlined, except for pAttGuide sequences, where the first three bases of the att-targeting spacer are underlined. FIG. 11B discloses SEQ ID NOS: 5792-5801, respectively, in order of appearance.

FIG. 12. Comparing spacers and protospacers in relation to reading frame. The four major att-site targeting spacers are compared to the protospacer (target) in each host; ffs, guaC, yciA, and rsmJ. The percentage of mismatches is indicated by position in the spacer comparing unique spacer-protospacer combinations (related to a diagram of the guide RNA showing the predicted flipped-out 6th position in red). The amino acid sequence is indicated to relate to the coding sequence (Note that ffs is functional as an RNA and the yciA gene is encoded on the opposite strand of guaC and rsmJ). The consensus sequence for the unique spacers and protospacers are indicated as Weblogos. The total number of mismatches per spacer-protospacer is indicated excluding the 6^{th} position which is flipped-out in the cascade complex in I-F systems. The number included in the tabulations is indicated (n). FIG. 12 discloses SEQ ID NOS: 5771-5773, respectively, in order of appearance.

FIG. 13. Elements with shortened spacers and their insertion positions. (a) ffs-integrated elements (SEQ ID NOS: 5737, 5738, 5774-5780, 5778, 5781, and 5780, respectively, in order of appearance), (b) araC-like integrated elements (SEQ ID NOS: 5782-5785, 5782-5784, 5786-5788, 5784, 5789, 5787, 5790, 5784, and 5791, respectively, in order of appearance). Features are indicated as in FIG. 2b. Arrays are shown for each element, with repeat sequences marked in dark grey, spacers marked in light grey, and conserved Cas6 binding motifs are underlined.

FIG. 14. Schematic representation of elements inserted downstream of pare. Similarity tree of TniQ proteins indicates that Parashewanella curva C51 has representatives that group with elements that target the parE att site and elements that use the I—F3 CRISPR-Cas system. In cases where two TniQs are found in the element, the one used for the similarity tree is indicated with highlighting.

FIG. 15. Cartoon depicting enzymes in association with a processed guide RNA (top) and illustrative upstream and downstream repeat sequences, a matched spacer, 3′ and 5′ handles segments of a repeat, spacer, repeat segment of a CRISPR array. A graphical depiction of nucleotides is included. A generic Type I system is shown. The S proteins are Small Subunit proteins, which are not present in IF-3 systems. In I—F3 systems, the Cas8 and Cas5 proteins are present in a fusion protein.

FIG. 16. Graphical depiction of transposition efficiency as determined using a mate out assay using the experimental approach as described in FIGS. 3B and 9A with the F plasmid lacZ target with a single guide RNA, lacZ4 spacer (see also, FIG. 3E). Guide RNAs contained atypical repeats from A. salmonicida S44. The 854GC construct contains a fusion of TnsA and TnsB proteins, with a deletion of HG at the C terminus of the TnsA protein, and an insertion of an A at the deletion site. The TnsA-TnsB 855GC contains a fusion of TnsA and TnsB proteins, with a deletion of HG at the C terminus of the TnsA protein, and an insertion of an R at the deletion site. The K. ocytoca linker construct contains a fusion of TnsA-TnsB proteins, separated by insertion of an 8 amino acid linker from K. ocytoca, as described below. The NLS-Strep construct contains a fusion of TnsA-TnsB in which the two protein segments are separated by, in a contiguous sequence in an N—C terminal direction, a GSG linker, a nuclear localization signal, a Strep affinity tag, and another GSG linker. The TnsABC vector expresses unfused TnsA and TnsB and TnsC proteins as a control. All of the experiments included the TniQ and Cascade proteins, which are further described herein. The data demonstrate that removal of certain amino acids (i.e., the HG), addition of amino acids (e.g., A and R), addition of a tag (e.g., the Strep tag) and addition of linkers (e.g., GSG and the K. ocytoca linker) are tolerated, and the described systems retain their transposition function.

### DETAILED DESCRIPTION

The present disclosure provides compositions and methods that relate to CRISPR systems for use in DNA modification. In particular, the disclosure provides guide RNAs (gRNAs) and expression vectors encoding the gRNAs, wherein the gRNAs comprise atypical repeat sequences (e.g. sequences that are RNA equivalents of atypical repeat sequences, such as those found in CRISPR arrays), as further described below. The gRNAs may also include truncated spacers. The gRNAs cooperate with proteins to form systems for use in enhanced DNA editing.

Unless defined otherwise herein, all technical and scientific terms used in this disclosure have the same meaning as commonly understood by one of ordinary skill in the art to which this disclosure pertains.

Every numerical range given throughout this specification includes its upper and lower values, as well as every narrower numerical range that falls within it, as if such narrower numerical ranges were all expressly written herein.

The disclosure includes all polynucleotide and amino acid sequences described herein. Each RNA sequence includes its DNA equivalent, and each DNA sequence includes its RNA equivalent. Complementary and anti-parallel polynucleotide sequences are included. Every DNA and RNA sequence encoding polypeptides disclosed herein is encompassed by this disclosure. Amino acids of all protein sequences and all polynucleotide sequences encoding them are also included, including but not limited to sequences included by way of sequence alignments. Sequences of from 80.00%-99.99% identical to any sequence (amino acids and nucleotide sequences) of this disclosure are included.

The disclosure includes all polynucleotide and all amino acid sequences that are identified herein by way of a database entry. Such sequences are incorporated herein as they exist in the database on the filing date of this application or patent. Unprocessed (e.g., RNAs that are not trimmed by described CRISPR proteins) and processed RNA polynucleotides (e.g., RNAs that are trimmed by the described CRISPR proteins) are included in this disclosure. The disclosure includes all of the sequences presented the sequence listing and figures, longer sequences comprising each of those sequences, e.g., sequences that comprise or consist of the described sequences with additional sequences at the 5′ and 3′ ends, and all contiguous segments of the described sequences. In embodiments, the described gRNA sequences are 28 to 37 nucleotides long, including but not limited to all atypical repeat sequences and spacer sequences, including but not limited to truncated spacer sequences. In embodiments, the gRNAs may comprise a spacer that is 29 nucleotides in length. Combinations of distinct sequences and segments thereof are included in this disclosure. RNA equivalents (e.g., replacement of T with U) for each sequence is also included, as are gRNAs that comprise such RNA equivalents, regardless of any additional sequences, including spacers. Expression vectors encoding any one or combination of repeat sequences described herein, are included. cDNA sequences of the sequences are included. Where a gene is described, the disclosure includes the protein encoded by the gene.

Any RNA polynucleotide of this disclosure may be initially transcribed as a guide RNA precursor, including but not limited to a crRNA, and may be transcribed from a DNA template that includes only one repeat-spacer-segment, or more than one repeat-spacer-segment, the latter of which includes but is not limited to all or a portion of CRISPR array, e.g., a segment of DNA that encodes more than one repeat-spacer-repeat segment. The disclosure also includes hybrid arrays, wherein at least one or some repeat sequences comprise atypical repeats as discussed below, whereas other repeats may be the same as one or more reference repeats, as also discussed below.

It is expected that guide RNAs (referred to from time to time as “gRNAs”) of this disclosure may be suitable for use in a number of CRISPR systems. The gRNAs of this disclosure include but are not limited to sequences that are expressly described herein by way of complete sequences that are the RNA equivalent of DNA repeat sequences, or such sequences that may vary in nucleotide position at certain positions as described herein. The disclosure also includes gRNAs and uses thereof that include gRNA that can be made according to methods described herein.

In embodiments, gRNAs of this disclosure may function with any of the Class 1 or 2 CRISPR systems. In embodiments, gRNAs of this disclosure are used with CRISPR systems, e.g., CRISPR systems initially found in bacteria or archaea, that include transposon proteins. In embodiments, the gRNAs of this disclosure are used with type I-F or Type I-B CRISPR-Cas systems. In embodiments, the disclosure provides gRNAs for use in CRISPR systems that are associated with Tn7-like transposons. In particular, bacterial genomes shows that many Tn7-like transposons contain ‘minimal’ type I-F CRISPR-Cas systems that contain of fused cas8f and cas5f, cas7f and cas6f genes, and a short CRISPR array. In embodiments, gRNAs of this disclosure are used with I-F CRISPR/Cas elements. Such systems, along with additional components described herein, provide for representative uses of the gRNAs that are at least one aspect of this disclosure.

Regardless of the type of CRISPR system used, recent analysis of CRISPR-facilitated genetic editing, including but not limited to insertions, indicates all insertions can be explained by guide RNAs. Further, families of conserved insertion sites that are found in chromosomes and originate from certain CRISPR systems used multiple times include attachment sites, abbreviated “att” sites.

In embodiments, the guide RNAs include repeat sequences that are the RNA equivalents of segments of repeat sequences identified in certain CRISPR arrays as having mutations, relative to other repeats in the same arrays, and such repeat sequences are referred to herein as from time to time as “atypical.” “RNA” equivalent as used herein means the RNA polynucleotide has the same sequence and same orientation as a described DNA sequence, except for the conventional substitution of uracil for thymine in the RNA polynucleotide.

Without intending to be bound by any particular theory, it has previously been considered that “older” repeats, e.g., those that flank less recently acquired spacers, have mutations that impede the function of guide RNAs that include the repeats and less recently acquired spacer sequences. However, as revealed in the present disclosure, it is considered that gRNAs comprising atypical repeats (which may or may not flank truncated spacers) may be preferentially complexed and processed by certain Cas enzymes, such as Cas5 and Cas6, which recognize 5′ and 3′ repeats (relative to a 5′->3′ oriented, intervening spacer) make more active guide RNA-Cas protein complexes. Alternatively, or additionally, the resulting guide RNA Cas protein complexes have enhanced activities that are not found in complexes made with the typical repeats.

In embodiments, the disclosure provides RNA polynucleotides (e.g., gRNAs) for use in CRISPR-based modification of DNA, as further described herein. In embodiments, the RNA polynucleotides comprise contiguously in a 5′ to 3′ orientation the following components, listed for clarity as A, B and C:

A) A 5′ end segment comprising a first RNA sequence that is the RNA equivalent of, or is transcribed, from an atypical first repeat sequence in a guide-RNA encoding DNA template, including but not limited to a CRISPR array. In embodiments, the 5′ end segment of the guide RNA that is, for example, derived from a repeat sequence, when in operation (e.g., during DNA binding of an RNA-protein complex to facilitate, for example, insertion of a DNA template) comprises or consists of 8 nucleotides.

B) An RNA sequence for DNA targeting (a targeting sequence, e.g., a spacer), wherein the targeting sequence is complementary to a protospacer in the DNA, and wherein the spacer may have a nucleotide length as further described herein.

C) A 3′ end segment comprising a second RNA sequence that is the RNA equivalent of, or is transcribed from, a second atypical repeat sequence in the guide RNA encoding DNA template, wherein optionally the 3′ end segment comprises or consists of 20 nucleotides. But additional nucleotides can be included, as further described below.

The described RNA polynucleotides, e.g., the described guide RNAs, comprise a spacer sequence, including but not limited to a truncated spacer sequence, that may be selected by a user of the described system, to direct the CRISPR system to a selected location in a DNA substrate, thereby facilitating insertion of any desired DNA template at a predetermined location.

In embodiments, an RNA polynucleotide is a recombinant RNA polynucleotide. A “recombinant” polynucleotide means an RNA polynucleotide that has been experimentally changed relative to a naturally occurring RNA polynucleotide. Thus, a recombinant RNA polynucleotide has been engineered to, for example, include a selected spacer to target a DNA sequence. A recombinant RNA polynucleotide may also include one or more atypical repeats that have been placed in the context of the selected spacer. Recombinant polynucleotides can include RNAs that are expressed from an expression vector designed to encode the desired RNA, or may be chemically synthesized. Recombinant RNA polynucleotides may also include modifications that are further described herein. The disclosure also includes recombinant DNA molecules that, for instance, encode an RNA polynucleotide and/or a protein described herein.

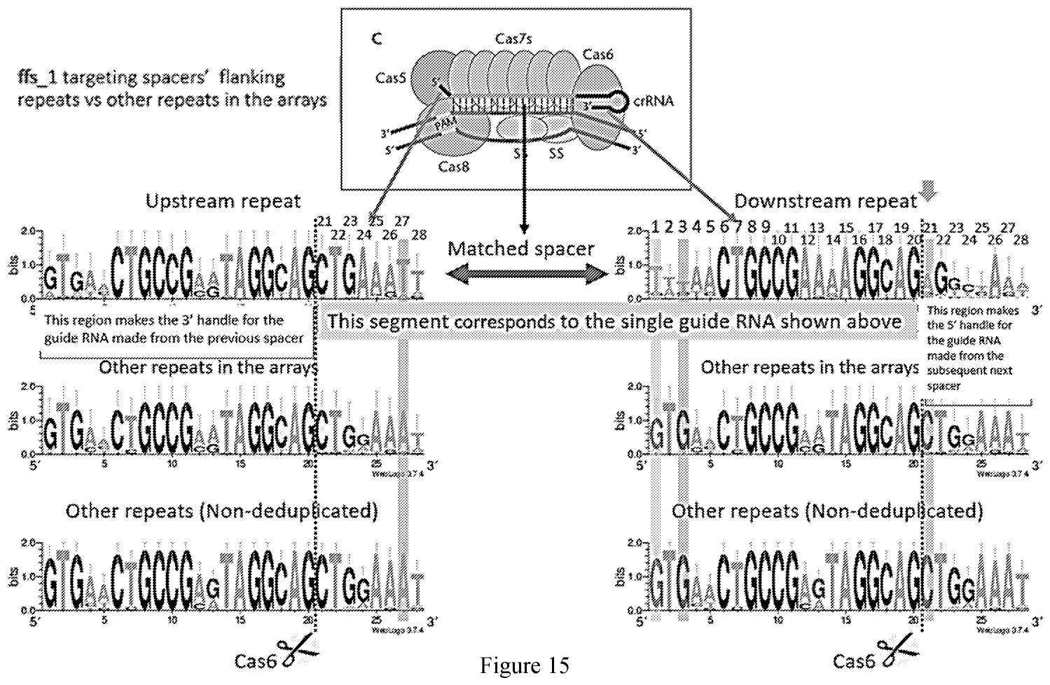

Certain aspects of the disclosure are illustrated generally by reference to FIG. 15. FIG. 15 shows representative “upstream” and “downstream” repeat sequences (e.g., 5′ and 3′ of an intervening spacer) which, in the natural array setting, is derived from a protospacer with appropriate protospacer adjacent motif (PAM). The DNA sequence is shown in the 5′->3′ orientation, and encodes a guide RNA that can form a complex with the indicated Cas proteins, as shown in the inset. For simplicity, only a single strand of DNA comprising a repeat-spacer-repeat segment of a CRISPR array is shown. The sequence shown in FIG. 15 is identical to the guide RNA, except each T is replaced with a U. The segment of the DNA that encodes the segment of the guide RNA that associates as a single unit with the CRISPR proteins is labeled “This region makes corresponds to the guide RNA shown above.” Thus, the described region illustrates what will be processed as a single guide RNA, and its interaction with a DNA template. Above the Matched Spacer is a cartoon depiction of the processed single guide RNA (labeled “crRNA”), with the relative location of the PAM in the double stranded DNA target. The Cas6 cleavage sites are indicated by the scissors. In FIG. 15, the nucleotides, as numbered with respect to repeats in the array, illustrate the 5′ and 3′ segment boundaries that flank the intervening spacer. RNA polynucleotides for use as guide RNAs according to this disclosure comprise an RNA sequence targeted to a DNA substrate as shown in the “Matched Spacer” segment. The guide RNAs provided by this disclosure comprises RNA nucleotide sequences that are the RNA equivalents to the upstream and downstream repeats, with a variable nucleotide positions illustrated by the relative size of the nucleotides, using FIG. 15 as a non-limiting illustration.

With respect to FIG. 15, RNA polynucleotides used in the CRISPR-based DNA modification techniques as described herein may be produced from a double-stranded DNA template that includes at least one repeat-spacer-repeat sequence, wherein FIG. 15 shows a single DNA sequence that includes representative and non-limiting upstream and downstream repeats in the CRISPR array.

Without intending to be bound by any particular theory, it is considered that nucleotides that define atypical repeats as described herein, at least portions of which may be incorporated into guide RNAs of this disclosure, affect the function of the CRISPR systems described herein. However, and again without intending to be bound by theory, it is also considered that nucleotides present in the upstream and downstream atypical repeat sequences in the DNA may influence performance of the CRISPR-based modification of target DNA, even if such RNA equivalent nucleotides are not ultimately present in the processed guide RNA. For example, using FIG. 15 as a non-limiting illustration, in certain embodiments, a first 5′ end segment of a guide RNA of this disclosure may include only nucleotides 21-28 that correspond to the upstream repeat, but this segment, as well as nucleotides in the DNA template further upstream of nucleotide 21, may diverge from reference repeat sequences, and such diverged, atypical sequences, may also contribute to improved performance of the presently provided systems. Thus, in certain embodiments, any one or more of the nucleotides in the first (atypical) 5′/upstream segment repeat of an RNA polynucleotide of this disclosure may differ from a reference repeat in one or any combination of nucleotide positions 21, 22, 23, 24, 25, 26, 27 or 28 of the upstream repeat. In embodiments, only 1, 2, 3, 4, 5, 6, 7, or all 8 such nucleotides are changed relative to a reference repeat. In certain embodiments, nucleotide 21 in the upstream repeat may be the same as, or different from, a reference repeat. The same applies to nucleotide locations further upstream in the atypical repeat, e.g., atypical nucleotides in any one or combination of positions 22-41 of the upstream repeat may affect, and improve, the function of a system of this disclosure. This is considered to be the case, even if such atypical nucleotides are not present in the sequence of a particular guide RNA after it is processed and functions in modification of a sequence dictated at least in part by the targeting DNA segment. Likewise, a 3′ end segment (e.g., a “downstream” segment) of a guide RNA of this disclosure may include nucleotides 1-20 of the downstream repeat, which can include at least some nucleotides that are different from a reference downstream repeat, but the disclosure includes RNA polynucleotides that may extend beyond nucleotide 20 in the downstream repeat.

In certain embodiments, the 3′ segment will generally, but not necessarily always, include a G as its 3′ terminal nucleotide as a component of a functional guide RNA, e.g., a guide RNA as depicted in the cartoon of FIG. 15. In certain embodiments, nucleotide changes in the downstream repeat either retain the reference repeat sequence in nucleotides 6-9 and 16-20, such as to facilitate formation of an appropriate 3′ hairpin structure, as shown in the cartoon of FIG. 15. However, the disclosure includes changes in the nucleotides 6-9 and 16-20 in the downstream repeat relative to a reference repeat, provided the changed nucleotides are collectively capable of forming a hairpin structure that is believed to be required for guide RNA processing. The disclosure thus includes making RNA polynucleotides that will function as processed guide RNAs from templates that include atypical nucleotides in positions 21-28 of the downstream repeat in the DNA template, even if the RNA equivalents of such sequences are not present in the processed RNA that functions to modify the intended target DNA.

In certain embodiments, the RNA sequence that targets a DNA targeting sequence (e.g., a spacer) is complementary to a protospacer sequence. The DNA targeting sequence is selected for inclusion in the RNA polynucleotide such that the RNA polynucleotide is suitable for use in CRISPR-based modification of a known DNA target sequence comprising a sequence that is complementary to the targeted DNA sequence in the RNA polynucleotide. In embodiments, the CRISPR modification of DNA using the described RNA polynucleotide as a guide RNA comprises introduction of a transposable element into the DNA that is part of a chromosome or a plasmid.

In non-limiting examples, in the 5′ end segment, and/or in the 3′ end segment of the RNA polynucleotide, at least one nucleotide within nucleotides positions 1-4 of the nucleotide 5′ end or 3′ end segment sequence is changed in the first and/or second sequence, relative to the same nucleotide position in the reference repeat sequence. Non-limiting illustrations of locations of nucleotide variations in atypical repeats are shown in the figures of this disclosure, and are provided in the sequence listing. The 5′ and 3′ end sequences may vary in 1-10 positions, inclusive, relative to the reference repeat positions. In embodiments, the ribonucleoprotein complex comprising a described RNA polynucleotide is present in a complex with one or a combination of Cas5, Cas6, Cas7, and Cas8. Such a complex may be in vitro or in vivo, such as in a prokaryotic or eukaryotic cell.

In embodiments, the 5′ end segment and 3′ end segments of the described RNA polynucleotides comprise palindromic sequences that are the same, or are different, from palindromic sequences in the reference repeat sequence(s). In embodiments, the first and/or second reference repeat sequence is the same as a repeat sequence present in a bacterium, or in Archaea, wherein the repeat sequence in the bacterium is contiguous with the last spacer acquired by the organism, or with a spacer that was acquired less recently than another spacer in the same array. Proteins suitable for use with the described guide RNAs are further described below.

Expression vectors encoding an RNA polynucleotide that comprise atypical repeats (e.g., RNA sequences that are RNA equivalents of atypical repeats or portions of such repeats) described herein, or identified by a method described herein, are included in the disclosure. In embodiments, the disclosure includes RNA polynucleotide transcribed from such an expression vector, wherein the RNA polynucleotides may be isolated and/or purified. Cells comprising such RNA polynucleotides and expression vectors encoding them are included.

In non-limiting embodiments, the proteins used in the described system comprise at least one protein that is from, or derived from, one or more organisms that include I-F3b transposons. In embodiments, a protein is derived from an organisms by, for example, expressing the protein using an expression vector, or an mRNA that is produced by a user of a described system for modifying a DNA template, as further described herein. A protein derived from a naturally occurring protein may also have modifications, such as nuclear localization signals, and/purification tags.

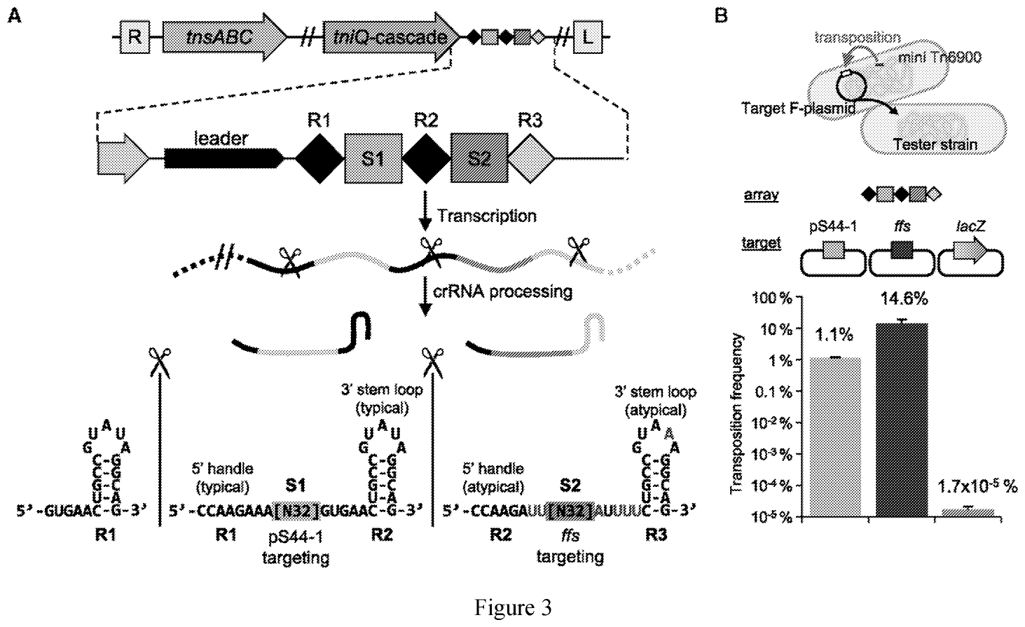

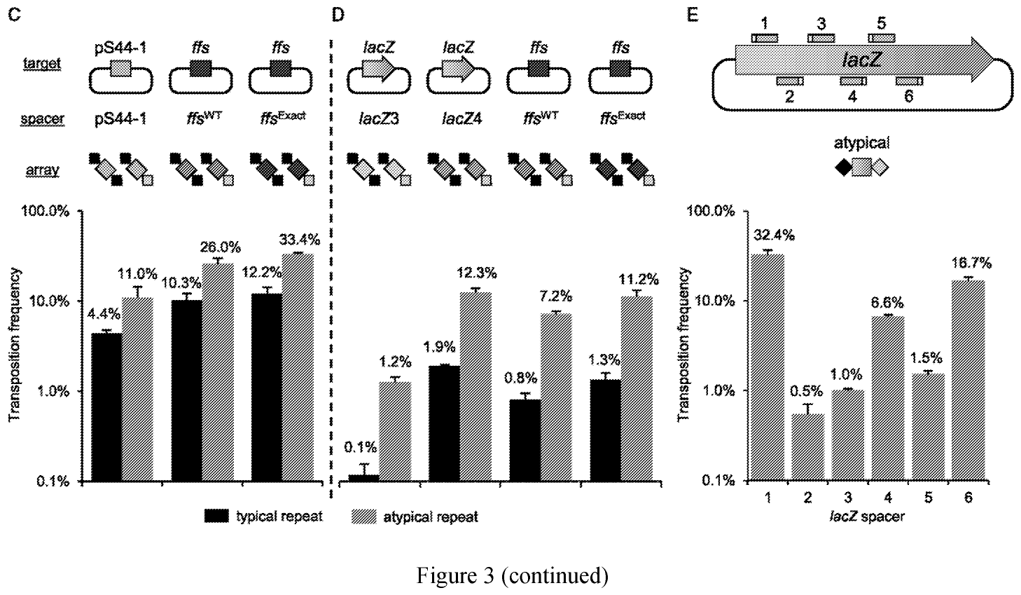

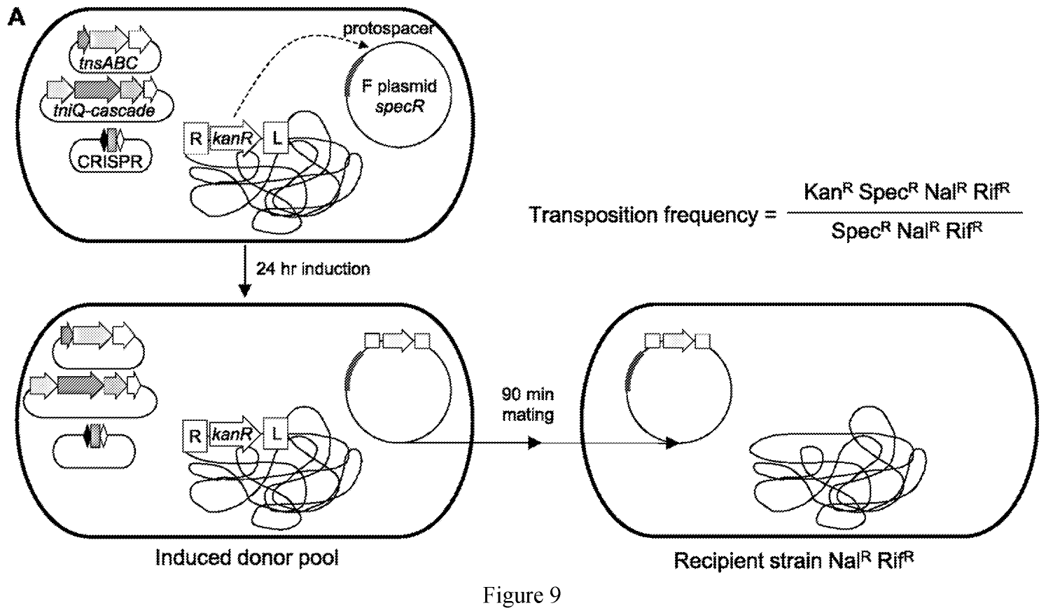

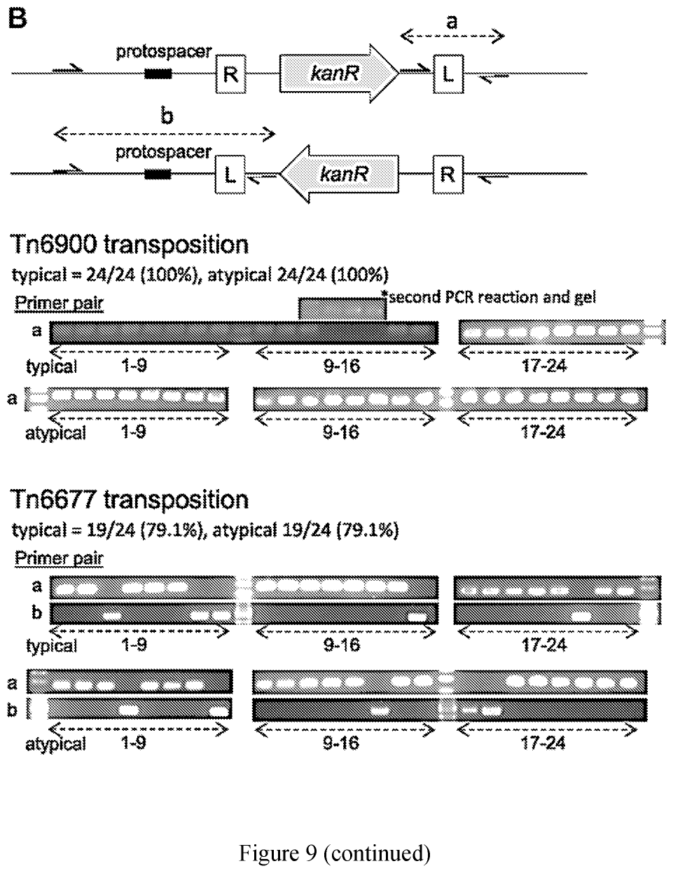

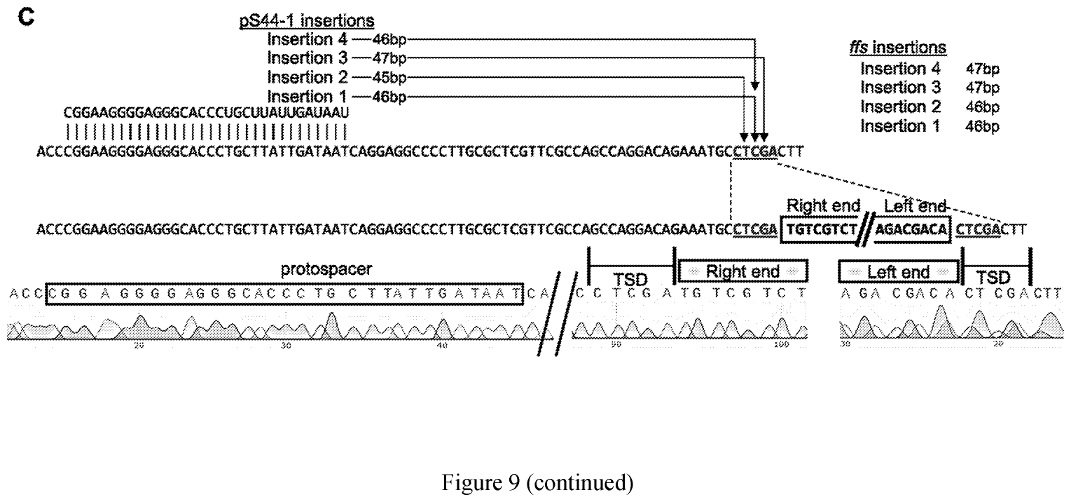

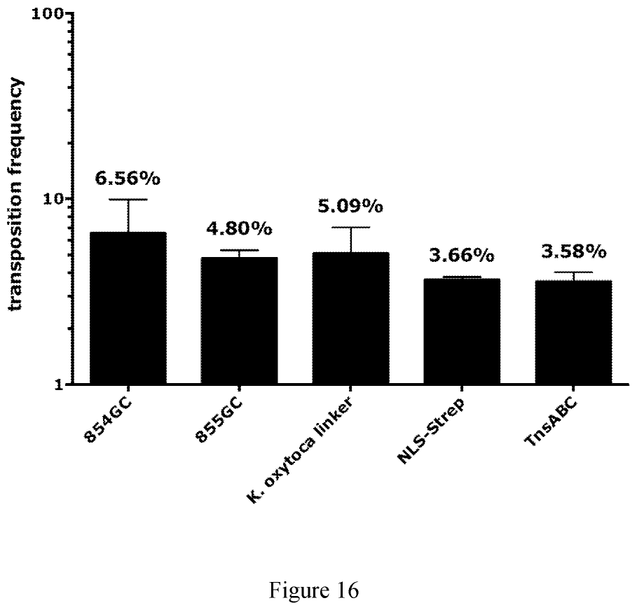

In embodiments, the one or more I-F3b proteins include I-F3b transposon proteins TnsA, TnsB, TnsC, TniQ, and I-F3b Cas proteins Cas8, Cas5, Cas7, and Cas6. One or more of the proteins may be fused together, with or without other proteins. In embodiments, Cas8 and Cas5 are present in a single fusion protein. In embodiments, TnsA and TnsB are present in a single fusion protein. In embodiments, TniQ is fused to another of the described proteins. In embodiments, TniC and TniQ are fused to one another. In embodiments, more than two of the described proteins may be present in a fusion protein. In embodiments, the proteins are fused to one another without linking amino acids. In alternative embodiments, linking amino acids can be included. In non-limiting embodiments, linking amino acids may form a flexible linker, and as such may comprise one or more amino acids to provide flexibility, such as Glycine rich linker. In non-limiting embodiments, the linker comprises Glycine and Serine. In an embodiment, the linker comprises 1-12 amino acids. In an embodiment, the linker comprises or consists of a GSG sequence. In embodiments, more than one linker can be used. In an embodiment, the linker comprises a segment of a protein from K. oxytoca. In an embodiment, the K. oxytoca linker comprises a contiguous sequence in an N-terminal to C-terminal direction that comprises all of KYA, QQN, SLF, ICS, and FP. In embodiments, a protein of this disclosure can include a tag, such as a purification tag, or other tags. In an embodiment, the tag comprises a Strep-tag. The amino acid sequence of a suitable Strep-tag is known in the art. In an embodiment, the Strep tag comprises in an N-terminal to C-terminal direction all of WSH, PQF, and EK. In embodiments, a protein of this disclosure comprises a nuclear localization signal (NLS). Suitable NLS sequence are known in the art. In an embodiment, the NLS comprises a contiguous sequence in an N-terminal to C-terminal direction that comprises all of PKK, KRK, and V. In an embodiment, a protein of this disclosure comprises a contiguous sequence that comprises in an N-terminal to C-terminal direction a linker, an NLS, a Strep-tag, and another linker, which may comprise the same sequence as the first linker. In an embodiment, a change to the described amino acid sequence includes a deletion of amino acids. In an embodiment, the terminal HG of, for example, TnsA encoded by Aeromonas salmonicida strain S44 plasmid pS44-1, may be deleted in a fusion protein. In embodiments, deletion of HG is accompanied by an insertion of an A or R at the deleted position. Representative fusion proteins have been constructed and determined to function for transposition in a standard mate out assay (said assay being described in conjunction with FIGS. 3B and 9A) with the F plasmid lacZ target with a single guide RNA, lacZ4 spacer (See FIG. 3E) in the context of atypical repeats. Results using such fusion proteins are presented in FIG. 16. In embodiments, proteins expressed from the described systems may be expressed from a coding sequence that includes a ribosomal skipping sequence. Ribosomal skipping sequences are known in the art and include, in non-limiting embodiments, the ribosomal skipping peptides T2A, P2A, E2A, and F2A

The described system also provides a DNA cargo sequence for use in insertion into a DNA substrate. The DNA cargo sequence can include left and right end transposon sequences. The transposon left and right end sequences may also be inserted with a DNA cargo. The DNA cargo sequence is inserted into a DNA substrate by cooperation of the described proteins and the targeting RNA to produce the DNA editing. Those skilled in the art will be able to understand the terms “left” and “right” transposon sequences, and recognize such sequences.

For use with I-F3b systems, the one or more I-F3b proteins may be obtained from, and modified if desired, from any of the organisms that encode I-F3b proteins that are described herein, including in the text, tables and figures. In embodiments, the I-F3b proteins are from, or are derived from, any member of a subset of the described I-F3b transposon containing organisms. In embodiments, an I-F3b protein is encoded by the genome of an organism with an attachment site downstream of the ffs gene encoding the signal recognition particle, and those that are downstream of the downstream of the rsmJ gene.

Suitable I-F3b proteins and organisms which use them are shown, for example, in the figures. Such organisms which include functional IF-3b systems may be also include other transposable elements.

In embodiments, the I-F3b proteins are functional with targeting RNAs that include spacer sequences that are shorter than 29 nucleotides, as further described herein, and can exhibit greater transposition frequency than that achieved with other I-F proteins, such as

IF-3a systems. Further, the increased transposition frequency may be influenced by the presence of one or more atypical repeat sequences, from which at least some nucleotides are included in the targeting RNA when it is operational in DNA editing. Thus, in embodiments, the DNA template from which the targeting RNA is produced comprises one or more atypical repeats, as further described below. Representative examples of atypical repeats are described herein, including in the figures, text and sequence listing. In embodiments, the targeting RNAs include repeat sequences that are the RNA equivalents of segments of repeat sequences in CRISPR arrays, such repeat sequences comprising atypical repeats.

As discussed above, it has previously been considered that older repeats that flank less recently acquired spacers include mutations that hamper the function of guide RNAs that include the repeats than less recently acquired spacer sequences. In embodiments, the older repeats are located at increasing distances from the AT-rich leader region of the CRISPR array where the repeats are originally inserted. Those skilled in the art will be able to recognize a CRISPR array leader sequence. Further, as is known in the art, new spacer/repeat combinations are added at the leader region near the cas6-encoding gene.

The present disclosure includes targeting RNAs, which may include precursors, e.g., longer RNA polynucleotides that are transcribed from a CRISPR array and are recognized and/or processed by Cas proteins, which utilize nucleotide sequences that are from repeat sequences that flank a spacer that is not the most recent spacer inserted into the CRISPR array. In embodiments, the targeting RNA is encoded by a template that includes one or more repeat sequences that flank the oldest spacer in a CRISPR array, or a spacer that was not the most recently acquired. In embodiments, a CRISPR array comprises at least two spacers, but the disclosure does not necessarily exclude use of atypical repeat sequences that may be present in a CRISPR RNA coding template that includes only one spacer.

In more detail, mutations due to DNA replication are more likely to arise and persist in repeats (in repeat-spacer-repeat segments) that are present in a CRISPR array for longer periods of time than their more recently acquired counterparts, resulting in degenerate repeat sequences that have previously been considered not functional for processing into viable guide RNA effector complexes. In particular, to the extent such mutated sequences have been observed, it has been assumed that degenerated repeats in the CRISPR array would, if used to produce a guide RNA, render the guide RNA non-functional, or less functional, in its CRISPR editing functions. However, the present disclosure demonstrates that in some cases the altered repeats are actually enhanced for editing function, at least insofar as certain types of transposon elements are involved in the process, as further described below. As noted above, these repeats with enhanced function are referred to as “atypical” repeats. In embodiments, degenerated repeats may be distinct from changes that arise from a recombination process, or by another homology-driven process where the DNA polymerase skips nucleotides on the template DNA of the repeat to the next repeat, also causing a deletion.

Again, and without intending to be constrained by any particular interpretation, it is considered that the atypical repeats may be preferentially complexed and processed by certain Cas enzymes, such as Cas5 and Cas6, which recognize 5′ and 3′ repeats (relative to a 5′->3′ oriented, intervening spacer) including but not necessarily limited to the RNA equivalent of the repeats, respectively to make more active guide RNA-Cas protein complexes. Alternatively, or additionally, the resulting guide RNA/Cas protein complexes have enhanced activities that are not found in complexes made with the typical repeats. Accordingly, in embodiments, the disclosure provides an RNA polynucleotide that can be used in CRISPR-based modification of DNA, the RNA polynucleotide comprising contiguously in a 5′ to 3′ orientation: a 5′ end segment comprising a first RNA sequence that is the RNA equivalent of an atypical first repeat sequence in a guide-RNA encoding DNA template, an RNA sequence for DNA targeting (a targeting sequence), wherein the targeting sequence is fully or at least partially complementary to a protospacer in the targeted DNA; and a 3′ end segment comprising a second RNA sequence that is the RNA equivalent of a second atypical repeat sequence in the guide-RNA encoding DNA template. The 5′ end segment, the 3′ end segment, or both, comprise one or more nucleotide changes relative to a first reference repeat sequence, and/or a second reference repeat sequence, respectively. In embodiments, the 5′ end segment and the 3′ end segment of the RNA polynucleotide each comprise one or more nucleotide changes relative to the first and second reference repeat sequences, respectively, and as further described below.

The reference sequence can be any suitable sequence that is different from the first and/or second repeat sequences that are components of the RNA polynucleotide and may include additional sequences found in repeats that are not necessarily included in a processed guide RNA that is used during DNA editing. In embodiments, the reference sequence comprises a repeat sequence that is immediately adjacent to a more recently acquired spacer in the same array as the atypical repeat. Thus, in embodiments, the 5′ end segment, the 3′ end segment, or both, in a targeting RNA, each comprise one or more nucleotide changes relative to the first and second reference repeat sequences, respectively. Thus, the disclosure includes use of repeat sequences that flank earlier acquired spacers. In this regard, and as is known in the art generally for certain CRISPR systems, repeats in the CRISPR array encode the guide RNA “handles” that are bound by Cas proteins, the guide RNAs being processed from a crRNA.

A non-limiting illustration of the processing of a crRNA that includes typical and atypical repeats is shown in FIG. 3, which is in addition to FIG. 15, and other figures of this disclosure. In FIG. 3A, bottom panel, the first R1 on the left shows the 5′ end of an unprocessed CRISPR array transcript. The second R1 from the left shows a 5′ handle transcribed from a typical repeat and its cleavage site shown by the first scissors and vertical line. S1 shows the location of a representative 32 nucleotide spacer that was more recently acquired in the CRISPR array, relative to the S2 spacer. The second R2 shows a typical 3′ stem loop. The second scissors and vertical line show the location of cleavage to produce the 3′ end of a first, e.g., more recently acquired spacer, and downstream repeat with a typical 3′ stem loop. To the right of the second scissors shows an atypical 5′ handle that is produced by the cleavage illustrated by the second scissors, followed by a later acquired spacer S2, and an atypical 3′ stem loop designated by R3. Differences between the repeat-spacer-repeat segments are apparent in the two UU nucleotides preceding S2, the A immediately following S2, the UUU sequence prior to the first strand of the stem loop, and the A in the fourth position of the atypical loop portion of the stem loop. FIGS. 3B, 3C and 3D provide graphical representations of data comparing use of targeting RNA that is transcribed from the described earlier and later acquired spacers. These data show that the targeting RNA transcribed and processed from the template that includes atypical repeats can facilitate enhanced transposition of a DNA element, relative to the targeting RNA that is transcribed from the segment of the template that includes typical repeat sequences. Thus, the disclosure demonstrates that use of a targeting RNA that is transcribed from a template that includes atypical repeats provides a beneficial effect on transposition efficiency. More discussion of FIG. 3 is provided in the Examples below.

Accordingly, use of targeting RNAs that are transcribed from a DNA template that includes atypical repeats may improve the function of any guide-RNA directed CRISPR system, and while the disclosure illustrated certain advantages of using the described gRNAs with Type IF-3b systems, the disclosure includes use of atypical repeats with any suitable CRISPR system, including but not limited to any Tn7-CRISPR/Cas elements, including but not limited to any I-F elements, and Type III, II, IV, V, VI systems, Type 1 and Type 2 CRISPR systems, Cas12K and multiple Type I-B systems. Further, the described atypical repeats may be used with any other Cas enzymes that can recognize the described handles. Such systems may include altered spacers, such as shortened spacers. In this regard, the present disclosure expands the demonstration of enhanced function of atypical repeats by demonstrating that targeting RNAs that are transcribed from a template that includes atypical repeats can be effective in increasing transposition frequency when used with Cas12K and multiple Type I-B systems. Further, enhanced transposition can be achieved, such as with I-F3b systems, but when shorter spacers (which may be accompanied by one or two atypical repeats) than those shown in FIG. 3 are used. For instance, while FIG. 3 depicts 32 nucleotide spacers ([N32]), the present disclosure includes use of shorter spacers to enhance transposition efficiency, which in embodiments is performed using an I-F3b system. A “system” as used herein means a combination of proteins and a guide RNA that are together necessary and sufficient to achieve DNA modification, non-limiting examples of which are discussed herein.

Notwithstanding the foregoing description, it is considered that the use of the described guide RNAs are suitable in one embodiment, for use with IF-3b systems, as described further herein. Additionally, the disclosure provides demonstrations that use of the described IF-3b systems exhibits increased transposition efficiency, relative to a control, such as an IF-3a system. Accordingly, in embodiments, the disclosure provides for use of the described guide RNAs, which may comprise and/or be transcribed from a CRISPR array that comprises at least one atypical repeats and may also comprise a shortened spacer.

It should be noted the sequence listing included as a part of this disclosure includes spacers from certain organisms that are only 31 nucleotides long. It is considered that certain systems use spacers that are generally 32 nucleotides long, but length variations can be present and still not provide enhanced transposition in the same manner as the truncated spacers of this disclosure. Thus, in embodiments, the present disclosure provides targeting RNAs that comprise spacer sequences may be fewer than 29 nucleotides in length. In this regard, targeting RNAs with shorter (e.g., 18-20 nucleotides) spacers are shown to have reduced or no detectable transposition function when used with, for example, I-F3a systems (Klompe et. al. 2019a).

A non-limiting demonstration showing that truncated spacers are functional with I-F3b systems is provided the figures. Thus, the disclosure provides an unexpected advantage in using targeting RNAs that are transcribed from templates that include atypical repeats, and using truncated spacers. The disclosure thus includes targeting RNAs that are transcribed from templates that include one or two atypical repeats and optionally, a truncated spacer. In embodiments, a guide RNA of this disclosure may include a segment transcribed from only one atypical repeat, or more than segment transcribed from an atypical repeat, wherein each segment includes a sequence that is the same as the atypical repeat. In embodiments, a guide RNA of this disclosure comprises more than one copy of the same atypical repeat. In embodiments, a guide RNA of this disclosure may include two atypical repeats flanking the same, or different spacers. In embodiments, the guide RNA may contain only one spacer, or more than one copy of the same spacer, or two or more different spacers. The guide RNAs are different from those produced naturally at least because the selected spacer does not appear in nature in the context of atypical repeats. The guide RNAs of the disclosure may also be different from those that appear in nature due to having at least a segment that is transcribed from an atypical repeat that is configured to operate with a selected spacer that was not encoded in an endogenously occurring CRISPR array.

In embodiments, a spacer of this disclosure may consist of 18, 19, 20, 21, 22, 23, or 24 nucleotides. In embodiments, a spacer comprises 1, 2, 3, 4, or 5 nucleotides that is/are transcribed from what is designated as an atypical repeat sequence in a CRISPR array, as described further herein. In embodiments, the 5′ end segment and 3′ end segments of the described RNA polynucleotides comprise palindromic sequences that are the same, or are different, from palindromic sequences in the reference repeat sequence(s). In embodiments, a spacer becomes atypical by reducing the size of a loop structure.

In embodiments, a handle of a guide RNA of this disclosure includes a 5′ nucleotide sequence that is CCUAC or a truncation of this sequence that is UAC, said sequences being encoded by the CRISPR array, which can include sequences encoding atypical repeat sequences. In embodiments, a CC sequence is part of a repeat sequence or part of a spacer sequence, or both, depending on which end of the spacer is being considered.

In more detail, guide RNAs (also referred to as targeting RNAs as discussed above) may be encoded by a CRISPR construct, including but not necessarily limited to a CRISPR array. In embodiments, a suitable guide RNA or a guide RNA precursor includes only one set of atypical repeats that flank one spacer sequence, or more than one set of the same or distinct atypical repeats that flank the same or distinct spacer sequences, may be used. It is expected that based on the present disclosure, a suitable targeting RNA can be produced with any guide RNA that is an aspect of this disclosure, e.g., the typical 5′ end or 3′ end that forms the guide RNA can be engineered to form a sequence that is the RNA equivalent of an atypical repeat.

As also discussed above, the described I-F3b systems use I-F3b CRISPR associated proteins (or Cas proteins) to make a complex (Cas proteins+guide RNA) to target DNAs that match the guide RNA sequence, with tolerance for certain mismatches between the spacer and a protospacer, as described further herein. Naturally occurring elements have evolved to use a subset of the I-F3b Cas proteins (Cas8/5f, Cas7f, and Cas6f) to process a cognate CRISPR array containing the guide RNA to target a cognate element to direct transposition adjacent to the DNA match to the guide RNA sequence, again with certain potential mismatches. I-F3b Cas8/5f (also referred to as Cas8-5) are naturally fused, and the present disclosure includes such fusion proteins. The I-F3b transposon proteins TnsA, TnsB, TnsC, and TnsD/TniQ recognize cognate “left” and “right” transposon DNA sequences that may be present in the targeted DNA substrate or in an insertion DNA template. As is known in the art, each left and right end sequence pair is ordinarily associated with a particular set of tnsA, tnsB and tnsC genes, and the left and right end sequences are considered “cognate” with respect to the particular tnsA, tnsB and tnsC cassette.

The disclosure includes intact proteins described herein, and also includes functional fragments thereof. A “functional fragment” means one or more segments of contiguous amino acids of a polypeptide described herein which retain sufficient capability to participate in target RNA programmed insertion of the DNA insertion template. In embodiments, a functional fragment may therefore comprise or consist of, for example, a core domain, a catalytic domain, a polynucleotide binding domain, and the like. A single domain, or more than one domain, can be present in a functional fragment.

In embodiments, combinations of naturally occurring proteins, wherein the proteins are from distinct sources, are used.

In embodiments, the compositions and methods of this disclosure are functional in a heterologous system. “Heterologous” as used herein means a system, e.g., a cell type, in which one or more of the components of the system are not produced without modification of the cells/system. A non-limiting embodiment of a heterologous system is any bacteria that is not Aeromonas salmonicida, including but not necessarily limited to Aeromonas salmonicida strain S44. In embodiments, a representative and non-limiting heterologous system is any type of E. coli. A heterologous system also includes any eukaryotic cell. In embodiments, the heterologous cell is a member of any group that does not endogenously use an I-F3b system. In embodiments, the disclosure includes adapting any proteins, repeat sequences, and guide RNA sequences, that are described in the sequence listing and the figures, which have a matched spacer length that is fewer than 31 nucleotides in length.

In embodiments, the presently described systems are used to insert a DNA insertion template to virtually any position in a bacterial genome, any episomal element, or a eukaryotic chromosome, in an orientation dependent fashion, but in certain instances may require a PAM sequence. In embodiments, the system is targeted via a targeting RNA to a sequence in a chromosome in a eukaryotic cell, or to a DNA extrachromosomal element in a eukaryotic cell, such as a DNA viral genome. Thus, the disclosure includes modifying eukaryotic chromosomes, and eukaryotic extrachromosomal elements, such as DNA in any organelle. Accordingly, the type of extrachromosomal elements that can be modified according to the presently described compositions and methods are not particularly limited.

In embodiments, systems of this disclosure include a DNA cargo for insertion into a eukaryotic chromosome or extrachromosomal element, or in the case of prokaryotes, a chromosome or a plasmid. Thus, instead of transposing an existing segment of a genome in the manner in which transposons ordinarily function, the disclosure provides for insertion of DNA cargo that can be selected by the user of the system. The DNA cargo may be provided, for example, as a circular or linear DNA molecule. The DNA cargo can be introduced into the cell prior to, concurrently, or after introducing a system of the disclosure into a cell. The sequence of the DNA cargo is not particularly limited, other than a requirement for suitable right and left ends that are recognized by proteins of the system. The right and left end sequences that are required for recognition are typically from about 90-150-bp in length. As is known in the art, such 90-150 bp length comprises multiple 22 bp binding sites for the I-F3b TnsB transposase in the element in each of the ends that can be overlapping or spaced.

The minimum length of the DNA cargo is typically about 700 bp, but it is expected that from 700 bp to 120 kb can be used and inserted. The disclosure provides for insertion of a DNA cargo without making a double-stranded break, and without disrupting the existing sequence, except for residual nucleotides at the insertion site, as is known in the art for transposons. In embodiments, the insertion of the DNA cargo occurs at a position that is from approximately 47, 48, or 49 nucleotides from a protospacer in the target (e.g., chromosome or plasmid) sequence.

Without intending to be constrained by any particular theory, it is considered that, other than a requirement for certain sequences to function with the I-F3b sequences as described herein, the presently provided systems are ambivalent with respect to the DNA sequence of the DNA insertion template. Accordingly, in embodiments, the DNA insertion template may be devoid of any sequence that can be transcribed, and as such may be transcriptionally inert. Such sequences may be used, for example, to alter a regulatory sequence in a genome, e.g., a promoter, enhancer, miRNA binding site, or transcription factor binding site, to result in knockout of an endogenous gene, or to provide an interval in the dsDNA substrate between two loci, and may be used for a variety of purposes, which include but are not limited to treatment of a genetic disease, enhancement of a desired phenotype, study of gene effects, chromatin modeling, enhancer analysis, DNA binding protein analysis, methylation studies, and the like.

In embodiments, the DNA sequence comprises a sequence that may be transcribed by any RNA polymerase, e.g., a eukaryotic RNA polymerase, e.g., RNA polymerase I, RNA polymerase II, or RNA polymerase III. In embodiments, the RNA that is transcribed may or may not encode a protein, or may comprise a segment that encodes a protein and a non-coding sequence that is functional. For example, functional RNAs include any catalytic RNA, or an RNA that can participate in an RNAi-mediated process. In embodiments, the functional RNA comprises all or a fragment of an siRNA, an shRNA, a tRNA, a spliceosomal RNA, or any type of micro RNA (miRNA), a snoRNA, or the like. In embodiments, the RNA that does not code for a protein encodes a long noncoding RNA (lncRNA).

In embodiments, the functional RNA may comprise a catalytic segment, and thus may be provided as a ribozyme. In embodiments, the ribozyme comprises a hammerhead ribozyme, a hairpin ribozyme, or a Hepatitis Delta Virus ribozyme. Such agents can be used, for example, to modulate any RNA to which they are targeted.

In embodiments, the DNA insertion template includes one or more promoters. The promoter may be constitutive or inducible. The promoter may be operably linked to a sequence that encodes any protein or peptide, or a functional RNA.

In embodiments, the DNA insertion template comprises one or more splice junctions. Thus, the insertion template may comprise a GU near a 5′ end of a coding sequence, and a branch site near the 3′ end of the coding sequence. In embodiments, the DNA insertion templates results in exon skipping, or it provides a mutually exclusive exon, or it provides an alternative 5′ splice junction as a donor site, or an alternative 3′ splice junction as an acceptor site, or a combination thereof. In embodiments, the DNA insertion template reduces or eliminates intron retention.

In embodiments, the DNA insertion template comprises at least one open reading frame, which may be operably linked to a promoter that is included with the DNA insertion template, or the DNA insertion template is linked to an endogenous cell promoter once integrated. The open reading frame, and thus the protein encoded by it, is not limited. In non-limiting embodiments, the DNA insertion template comprises an open reading frame that encodes a peptide, e.g., a peptide that can be translated and which may be, for example, from several to 50 amino acids in length, whereas longer sequences are considered proteins.

In embodiments, a protein encoded by the DNA insertion template includes a cellular localization signal, and thus may be transported to any particular cellular compartment. In embodiments, the encoded protein comprises a secretion signal. In embodiments, the encoded protein comprises a transmembrane domain, and thus may be trafficked to, and anchored in a cell membrane. In embodiments, the anchored protein may comprise either or both of an intracellular domain and an extracellular domain, and may accordingly be displayed on the cells surface, and may further participate in, for example, signal transduction, e.g., the protein comprises a surface receptor. In embodiments, a protein encoded by the DNA integrate template comprise a nuclear localization signal. In embodiments, a protein encoded by the DNA integrate template comprises one or more glycosylation sites.

In embodiments, the protein encoded by the DNA insertion template comprises at least one antigenic determinant, e.g., an epitope, and thus may be used to produce cells, such as antigen presenting cells, that may display a peptide comprising an epitope on the cell surface via MHC (e.g, HLA) presentation.

In embodiments, the protein encoded by the DNA insertion template encodes a binding partner, such as an antibody or antigen binding fragment of an antibody. In embodiments, the binding partner comprises an intact immunoglobulin, or as fragments of an immunoglobulin including but not necessarily limited to antigen-binding (Fab) fragments, Fab′ fragments, (Fab′)₂ fragments, Fd (N-terminal part of the heavy chain) fragments, Fv fragments (two variable domains), dAb fragments, single domain fragments or single monomeric variable antibody domains, isolated CDR regions, single-chain variable fragment (scFv), and other antibody fragments that retain antigen binding function. In embodiments, one or more binding partners are encoded by the DNA insertion template and encode all or a component of a Bi-specific T-cell engager (BiTE), a bispecific killer cell engager (BiKE), or a chimeric antigen receptor (CAR), such as for producing chimeric antigen receptor T cells (e.g. CAR T cells). In embodiments, the binding partners are multivalent, and as such may include tri-specific antibodies or other tri-specific binding partners.

In embodiments, the DNA insertion template encodes a T cell receptor, and thus may encode both an alpha and beta chain T cell receptor, or separate DNA insertion template s may be used.

In embodiments, the DNA insertion template encodes an enzyme; a structural protein; a signaling protein, a regulatory protein; a transport protein; a sensory protein; a motor protein; a defense protein; or a storage protein. In embodiments, the DNA insertion template encodes a protein or peptide hormone. In embodiments, the DNA insertion template encodes hemoglobin. In embodiments, the DNA insertion template encodes all or a segment of dystrophin. In embodiments, the DNA insertion template encodes a rod or cone protein. In embodiments, the DNA insertion template encodes a selectable or detectable marker. In embodiments, the detectable marker comprises a fluorescent protein, such as green fluorescent protein (GFP), enhanced GFP (eGFP), mCherry, and the like. In embodiments, the DNA insertion template encodes an auxotrophic marker, such as for use in yeast. In embodiments, the DNA insertion template encodes one or more proteins that are involved in a metabolic pathway.

In embodiments, the DNA insertion template encodes a peptide or protein that is intended to stimulate an immune response, which may be a humoral and/or cell mediated immune response, and may also include a peptide or protein that is intended to induce tolerance, such as in the case of an autoimmune disease or an allergy. In embodiments, the DNA insertion template encodes a Toll-like-receptor (TLR), or a TLR ligand, which may be an agonist or an antagonistic TLR ligand.

In embodiments, the DNA insertion template comprises a sequence that is intended to disrupt or replace a gene or a segment of a gene. Thus, the disclosure includes producing both knock in and knock out gene modifications in cells, and transgenic non-human animals that contain such cells, as well as prokaryotic cells modified in a similar manner.

In embodiments, the transposable DNA cargo sequence is inserted into the chromosome or extrachromosomal element within a 5 nucleotide sequence that includes the nucleotide that is located 47 nucleotides 3′ relative to the 3′ end of the protospacer. In embodiments, a DNA cargo insertion comprises an insertion at the center of a 5 bp target site duplication (TSD). Thus, in non-limiting embodiments, a suitable guide RNA directs an editing complex to a DNA target comprising PAM that is cognate to the protospacer, so that precise integration of a DNA cargo can be achieved. In embodiments, the PAM comprises or consists of TACC or CC, NC, or CN (where “N” is any nucleotide).

The I-F3b transposon and I-F3b Cas genes, or those from any other suitable system, can be expressed from any of a wide variety of existing mechanism that can replicate separately in the cell or be integrated into the host cell genome. Alternatively, they could be expressed transiently from an expression system that will not be maintained. In certain embodiments, the proteins themselves could be directly transformed into the host strain to allow their function. The disclosure allows for multiple copies of distinct transposon gene cassettes, multiple copies of Cas genes, CRISPR arrays, and multiple distinct cargo coding sequences to be introduced and to modify genetic material in the same cell. In embodiments a first set of I-F3b genes tnsA, tnsB, tnsC, and one or more I-F3b tniQ genes, and I-F3b Cas genes cas8f, cas5f, cas7f, and cas6f, and a sequence encoding at least a first guide RNA that is functional with I-F3b proteins encoded by the Cas genes, wherein at least one of the first set of I-F3b transposon genes, the I-F3b Cas genes, or the sequence encoding the first guide RNA are present within and/or are encoded by a recombinant polynucleotide that is introduced into heterologous bacteria, or eukaryotic cells. The disclosure thus includes second, third, fourth, fifth, or more copies of distinct I-F3b transposon genes, I-F3b Cas genes, and distinct cargo coding sequences.

The delivery vector can be based on any number of plasmid, bacteriophage or another genetic element, when used in prokaryotes. The vector can be engineered so it is maintained, or not maintained (using any number of existing plasmid, bacteriophage or other genetic elements). Delivery of these DNA constructions in bacteria can be by conjugation, bacteriophage or any transformation processes that functions in the bacterial host of interest.

Modifications of this system may include adapting the expression system to allow expression in eukaryotic or archaeal hosts. In embodiments, for eukaryotic cells, the disclosure includes use of at least one nuclear localization signal (NLS) in one or more proteins. In general, a suitable NLS includes one or more short sequences of positively charged lysines or arginines exposed on the protein surface. In embodiments, a system of this disclosure is introduced into eukaryotic cells using, for example, one or more expression vectors, or by direct introduction of ribonucleoproteins (RNPs). In embodiments, expression vectors comprise viral vectors. In embodiments, a viral expression vector is used. Viral expression vectors may be used as naked polynucleotides, or may comprises any of viral particles, including but not limited to defective interfering particles or other replication defective viral constructs, and virus-like particles. In embodiments, the expression vector comprises a modified viral polynucleotide, such as from an adenovirus, a herpesvirus, or a retrovirus, such as a lentiviral vector. In embodiments, a baculovirus vector may be used. In embodiments, any type of a recombinant adeno-associated virus (rAAV) vector may be used. In embodiments, a recombinant adeno-associated virus (rAAV) vector may be used. rAAV vectors are commercially available, such as from TAKARA BIO® and other commercial vendors, and may be adapted for use with the described systems, given the benefit of the present disclosure. In embodiments, for producing rAAV vectors, plasmid vectors may encode all or some of the well-known rep, cap and adeno-helper components. In certain embodiments, the expression vector is a self-complementary adeno-associated virus (scAAV). Suitable ssAAV vectors are commercially available, such as from CELL BIOLABS, INC.® and can be adapted for use in the presently provided embodiments when given the benefit of this disclosure.

Further modification of this approach can include expression and isolation of the proteins required for this process and carrying out some or all of the process in vitro to allow the assembly of novel DNA substrates. These DNA substrates can subsequently be delivered into living host cells or used directly for other procedures. Thus, the disclosure includes compositions, methods, vectors, and kits for use in the present approach to DNA editing.

In one example, the disclosure provides a system for modifying a genetic target in bacteria and/or eukaryotic cells. The system comprises a first set of I-F3b transposon genes tnsA, tnsB, tnsC, one or more I-F3b tniQ, Cas genes cas8f cas5f cas7f, and cas6f and a sequence encoding a first guide RNA as described herein that is functional at least with proteins encoded by the I-F3b Cas genes, wherein at least one of the first set of transposon genes, the Cas genes, and/or or the sequence encoding the first guide RNA are present within and/or are encoded by a recombinant polynucleotide.

In embodiments, use of the described I-F3b systems exhibit a greater transposition frequency than transposition reference frequency. In embodiments, use of the described I-F3b systems exhibit a greater transposition frequency than a transposition frequency using the same set of proteins and guide RNA but where the proteins are from an I-F3a system. In embodiments, for instance in bacteria, transposition frequency can be determined using, for example, a bacteriophage (i.e. viral) vector that cannot replicate or integrate into the bacterial strain used in the assay. Therefore, while the viral vector injects its DNA into the cell, it is lost during cell replication. Encoded in the phage DNA is a miniature Tn7 element where the Right and Left ends of the element flank a gene that encodes resistance to an antibiotic, such as Kanamycin (KanR). If the transposon remains on the bacteriophage DNA the cell will still be killed by the antibiotic because the bacteriophage cannot be maintained in that particular strain of bacteria. However if the TnsA, TnsB, TnsC and other required I-F3b transposon proteins and nucleotide sequences described herein are added to the cell, transposition will occur because the transposon can move from the bacteriophage DNA into the chromosome (or plasmid) where it will be maintained and allow a colony of bacteria to grow that is antibiotic resistant. Therefore, when the number of infectious bacteriophage particles are in the assay is known, it permits calculation of a frequency of transposition as antibiotic resistant colonies of bacteria per bacteriophage used in the experiment. Thus, in embodiments, using one or a combination of the I-F3b proteins described herein increases transposition frequency. Accordingly, in some embodiments, one or more I-F3b proteins and guide RNA elements as described herein may be used to enhance CRISPR mediated insertion that is accompanied by the transposon-based constructs that are described herein.

In alternative embodiments, detectable markers and selection elements can be used. In embodiments, transposition frequency can be measured, for example, by a change in expression in a reporter gene. Any suitable reporter gene can be used, non-limiting examples of which include adaptations of standard enzymatic reactions which produce visually detectable readouts. In embodiments, adaptations of β-galactosidase (LacZ) assays are used. In embodiments, transposition of an element from one chromosomal location to another, or from a plasmid to a chromosome, or from a chromosome to a plasmid, results in a change in expression of a reporter protein, such as LacZ. In embodiments, use of a system described herein causes a change in expression of LacZ, or any other suitable marker, in a population of cells. In embodiments, transposition efficiency is determined by measuring the number of cells within a population that experience a transposition event, as determined using any suitable approach, such as by reporter expression, and/or by any other suitable marker and/or selection criteria. In embodiments, the disclosure provides for increased transposition, such as within a population of cells, relative to a control. As described above, the control can be any suitable control, such as a reference value, or any value using a control experiment with I-F3a transposon proteins. In embodiments, the reference value comprises a standardized curve(s), a cutoff or threshold value, and the like. In embodiments, transposition efficiency comprises use of a system of this disclosure to transpose all or a segment of DNA from one location to another within the same or separate chromosomes, from a chromosome to a plasmid, or from a plasmid or other DNA cargo to a chromosome. In embodiments, transposition efficiency is greater than a control value obtained or derived from transposition efficiency using the described system.

In one aspect, the disclosure provides a system for modifying a genetic target in one or more cells, the system comprising a first set of transposon genes tnsA, tnsB, tnsC, and tniQ, Cas genes cas8f, cas5f cas7f and cas6f and optionally an xre gene encoding a transcription regulator, or optionally one or more proteins encoded by one or more of said genes, and wherein optionally at least two of said proteins are within a fusion protein, and a sequence encoding an RNA polynucleotide comprising a sequence that is partially or fully an RNA equivalent of an atypical repeat. Wild type or modified genes, and proteins encoded by wild type or modified genes, may be used. For example, in non-limiting embodiments, the tnsA gene optionally comprises a change in sequence such that at least one amino acid in the TnsA protein encoded by the tnsA gene is changed relative to its wild type sequence. In embodiments, at least one of the following is true:

i) the tnsB gene comprises a change in sequence such that at least one amino acid in the TnsB protein encoded by the tnsB gene is changed relative to its wild type sequence or if the protein is used the protein comprises said change;

ii) the tnsC gene comprises a change in sequence such that at least one amino acid in the TnsC protein encoded by the tnsC gene is changed relative to its wild type sequence or if the protein is used the protein comprises said change.

In embodiments, a change in the TnsA protein comprises a change of Ala at position 125 of an Aeromonas salmonicida TnsA protein, wherein optionally the change is to an Asp, or is a homologous change in a homologous TnsA protein.

In another embodiment the disclosure provides a method comprising expressing an RNA polynucleotide as described above in cells comprising first transposon genes tnsA, tnsB, tnsC, and optionally at least one tniQ, Cas genes cas8f, cas5f, cas7f, and cas6f, and optionally xre, wherein optionally at least one of the first set of transposon genes or the Cas genes are present within a recombinant polynucleotide. In embodiments, spacer is in the RNA polynucleotide is targeted to a DNA segment in a chromosome or plasmid in the cells, which may comprise a protospacer and may be adjacent to a suitable PAM.

In another embodiment, the disclosure provides a method for identifying and using atypical repeat sequences and/or truncated spacer sequences that can be used as templates for producing RNA polynucleotides as described herein. This method comprises analyzing CRISPR arrays and determining repeat sequences flanking spacers in the CRISPR arrays, comparing repeat sequences flanking earlier acquired spacers to repeat sequences flanking later acquired spacers, determining differences between repeat sequences flanking the earlier and later acquired spacers, and designating the repeat sequences flanking the earlier acquired spacers that are different from the repeat sequences flanking the later acquired spacers as candidates for use in CRISPR-based DNA modification with improved efficiency, relative to CRISPR-based DNA modification using the RNA comprising segments that are RNA equivalents of repeat sequences flanking the later acquired spacers. The same approach applies to identifying truncated spacers, e.g., spacers that are shorter in nucleotide length than were previously believed to be non-functional, or exhibiting reduced function, relative to normal spacer lengths.

In embodiments, the method further comprises producing an RNA polynucleotide comprising the 5′ and 3′ ends that are RNA equivalents of the repeats flanking the earlier acquired spacers (and may include spacers that are shorter than previously used for targeting any suitable protospacer). In embodiments, this method further comprises using the described RNA polynucleotide in a CRISPR-based DNA modification. In embodiments, the method is such that the RNA polynucleotide comprises a substitution of the spacer in the analyzed CRISPR array with a distinct sequence targeted to a predetermined DNA sequence present in a chromosome or plasmid. The disclosure includes RNA polynucleotides produced according to the described method, and expression vectors that encode such RNA polynucleotides. In one embodiment, a library of atypical repeat sequences is provided. In embodiments, a library of expression vectors encoding RNA polynucleotides identified by a described method is provided.

In another embodiment, the disclosure provides a database comprising a plurality of entries, the entries comprising or consisting of repeat sequences flanking earlier acquired spacers identified according to a method of this disclosure, and thus also comprises RNA sequences that are complete or partial RNA equivalents of such repeat sequences. In embodiments, the disclosure includes selecting one or more repeat sequences from the database, and producing an expression vector encoding segments that are RNA equivalents of all or a portion of the one or more repeats, and/or producing an RNA polynucleotide comprising the one or more RNA equivalent sequences, which may or may not include a sequence targeted to any protospacer.

In another embodiment, the disclosure provides a kit for producing an expression vector for use in CRISPR-based DNA modification, the kit comprising a vector comprising one or more restriction endonuclease recognition sites configured for cloning a desired targeting DNA such that the targeting DNA is contiguous with one or more sequences that are RNA equivalents of repeat sequences identified according to a method of this disclosure, and/or any particular atypical repeat sequence that is described herein.

An RNA polynucleotide (e.g., a guide RNA) for use in CRISPR-based modification of DNA, the RNA polynucleotide comprising contiguously in a 5′ to 3′ orientation: A) A 5′ end segment comprising a first RNA sequence that is the RNA equivalent of, or is transcribed, from an atypical first repeat sequence in a guide-RNA encoding DNA template. In embodiments, the 5′ end segment of the guide RNA, when in operation is associate with CRISPR proteins (e.g., during DNA binding of an RNA-protein complex to facilitate, for example, insertion of a DNA template) comprises or consists of 8 nucleotides; B) an RNA sequence for DNA targeting (a targeting sequence, e.g., a spacer), wherein the targeting sequence is complementary to a protospacer in the DNA; C) and a 3′ end segment comprising a second RNA sequence that is the RNA equivalent of, or is transcribed from, a second atypical repeat sequence in the guide-RNA encoding DNA template, wherein optionally the 3′ end segment comprises or consists of 20 nucleotides, but additional nucleotides can be included, as further described below. The described RNA polynucleotides, e.g., the described guide RNAs, may comprise a spacer sequence that is selected by a user of the described system, to direct the CRISPR system to a selected location in a DNA substrate, thereby facilitation of insertion of a DNA template, which also may be selected by the user of the described system.

In embodiments, increased transposition frequency is believed to be influenced by the presence of one or more atypical repeat sequences, from which at least some nucleotides are included in the targeting RNA when it is operational in DNA editing. Accordingly, the disclosure demonstrates increased transposition efficiency using the I-F3b system, relative to transposition frequency using an IF-3b system with the same guide RNAs.

As discussed above, a representative IF-3b system includes the described guide RNAs, and proteins obtained or derived from Aeromonas salmonicida, including but not necessarily limited to Aeromonas salmonicida strain S44. Additional organisms that include IF-3b systems are provided in Table A. However, it is considered that non IF-3b systems, if present in any of these organisms, will not exhibit enhanced transposition when used with the described guide RNAs and CRISPR systems.

TABLE A
Representative organisms containing IF-3b systems.

| | | | SEQ ID |
|---|---|---|---|
| Genus | species | strain | NOS |
| Shewanella | piezotolerans | WP3 | 1-7 |
| Pseudoalteromonas | spongiae | UST010723-006 | 8-14 |
| Pseudoalteromonas | undina | NCIMB 2128 | 15-20 |
| Grimontia | indica | AK16 | 21-26 |
| Vibrio | parahaemolyticus | CFSAN007435 | 27-34 |
| Vibrio | vulnificus | VV4-03 | 35-40 |
| Vibrio | parahaemolyticus | 856404 | 41-46 |
| Vibrio | crassostreae | J5-20 | 47-52 |
| Vibrio | crassostreae | LGP7 | 53-58 |
| Vibrio | splendidus | UCD-SED7 | 59-64 |
| Vibrio | splendidus | UCD-SED10 | 65-70 |
| Pseudoalteromonas | rubra | SCSIO 6842 | 71-76 |
| Aliivibrio | sp. | 1S165 | 77-78 |
| Vibrio | parahaemolyticus | GCSL_R144 | 79-84 |
| Vibrio | cholerae | TP | 85-92 |
| Vibrio | parahaemolyticus | MAVP4 | 93-100 |
| Vibrio | parahaemolyticus | MAVP78 | 101-108 |
| Vibrio | parahaemolyticus | MAVP-112 | 109-116 |
| Vibrio | parahaemolyticus | CTVP34C | 117-124 |
| Vibrio | coralliilyticus | RE87 | 125-130 |
| Vibrio | lentus | 10N.286.46.A11 | 131-136 |
| Vibrio | breoganii | 10N.222.49.A8 | 137-141 |
| Vibrio | breoganii | 10N.222.51.A12 | 142-146 |
| Vibrio | tasmaniensis | 10N.261.51.E11 | 147-152 |
| Vibrio | breoganii | 10N.261.51.E6 | 153-157 |
| Vibrio | splendidus | ZS_138 | 158-163 |
| Vibrio | splendidus | ZS_90 | 164-169 |
| Vibrio | crassostreae | 28_O_19 | 170-175 |
| Vibrio | cholerae | 1 | 176-179 |
| Pseudoalteromonas | ruthenica | S3245 | 180-188 |
| Pseudoalteromonas | ruthenica | S2898 | 189-197 |
| Vibrio | parahaemolyticus | F10_3 | 198-203 |
| Vibrio | parahaemolyticus | B6_5 | 204-211 |
| Vibrio | cholerae | N2780 | 212-217 |
| Vibrio | mimicus | VM223 | 218-221 |
| Vibrio | crassostreae | 9ZC13 | 222-227 |
| Vibrio | sp. | J2-12 | 228-233 |
| Vibrio | sp. | J2-15 | 234-238 |
| Vibrio | cholerae | YB3G04 | 239-244 |
| Vibrio | cholerae | YB2A06 | 245-250 |
| Vibrio | fluvialis | FDAARGOS_104 | 251-254 |
| Vibrio | natriegens | ATCC 14048 | 255-260 |
| Vibrio | natriegens | CCUG 16373 | 261-263 |
| Vibrio | parahaemolyticus | CDC_K5276 | 264-271 |
| Vibrio | sp. | 10N.261.45.E1 | 272-279 |
| Pseudoalteromonas | sp. | A601 | 280-286 |
| Vibrio | parahaemolyticus | MAVP56 | 287-294 |
| Vibrio | parahaemolyticus | MAVP-90 | 295-302 |
| Vibrio | parahaemolyticus | MAVP-46 | 303-308 |
| Pseudoalteromonas | nigrifaciens | KMM 661 | 309-316 |
| Pseudoalteromonas | arctica | MelAa3 | 317-322 |
| | | | and |
| | | | 1303-1308 |
| Pseudoalteromonas | sp. | GutCa3 | 323-328 |
| | | | and |
| | | | 1743-1748 |
| Vibrio | lentus | 10N.286.46.A10 | 329-334 |
| Vibrio | lentus | 10N.261.45.E12 | 335-340 |
| Vibrio | lentus | 10N.261.46.A1 | 341-346 |
| Vibrio | lentus | 10N.261.51.F9 | 347-352 |
| Vibrio | vulnificus | 162 | 353-358 |
| Vibrio | vulnificus | 32 | 359-364 |
| Vibrio | sp. | E4404 | 365-367 |
| Klebsiella | oxytoca | 67 | 368-373 |
| Aeromonas | salmonicida | AJ83 | 374-379 |
| Vibrio | penaeicida | CAIM 285 | 380-386 |
| Vibrio | crassostreae | 16BF1_95 | 387-392 |
| Vibrio | crassostreae | 30_P_66 | 393-397 |
| Vibrio | kanaloae | 10N.261.48.E7 | 398-403 |
| Vibrio | cholerae | A12JL4W4 | 404-411 |
| Vibrio | cholerae | A120618Z1 | 412-417 |
| Marinomonas | polaris | DSM 16579 | 418-424 |
| Pseudoalteromonas | sp. | JB197 | 425-432 |
| Photobacterium | damselae | NCTC11646 | 433-439 |
| Pseudoalteromonas | nigrifaciens | NCTC10691 | 440-447 |
| Vibrio | vulnificus | A14 | 448-454 |
| Vibrio | vulnificus | 95-8-7 | 455-461 |
| Vibrio | parahaemolyticus | UCM-V493 | 462-467 |
| Vibrio | splendidus | FF_139 | 468-480 |
| Vibrio | splendidus | FF_139 | 468-480 |
| Vibrio | lentus | 10N.261.52.F1 | 481-486 |
| Vibrio | lentus | 10N.286.51.F12 | 487-492 |
| Vibrio | cyclitrophicus | 10N.286.54.E5 | 493-498 |
| Vibrio | crassostreae | 34_P_122 | 499-503 |
| Oleiphilus | messinensis | DSM 13489 | 504-518 |
| Pseudoalteromonas | sp. | JB197 | 425-432 |
| Vibrio | azureus | LC2-005 | 519-525 |
| Vibrio | splendidus | 10N.286.52.C6 | 526-531 |
| Vibrio | splendidus | 10N.261.46.B10 | 532-537 |
| Vibrio | sp. | EJY3 | 538-542 |
| Vibrio | cyclitrophicus | 1F97 | 543-547 |
| Photobacterium | phosphoreum | FS-1.2 | 548-554 |
| Vibrio | tapetis | — | 555-560 |
| Vibrio | anguillarum | NCTC12159 | 561-566 |
| Vibrio | sp. | 10N.261.45.A1 | 567-574 |
| Vibrio | sp. | 10N.261.45.A6 | 575-582 |
| Vibrio | lentus | 10N.261.48.C12 | 583-588 |
| Vibrio | breoganii | 10N.261.48.E3 | 589-595 |
| Photobacterium | leiognathi | ajapo.3.1 | 596-601 |
| Halomonas | sp. | Soap Lake #6 | 602-609 |
| Pseudoalteromonas | sp. | S1727 | 610-615 |
| Pseudoalteromonas | sp. | S558 | 616-622 |
| Vibrio | sp. | BEI176 | 623-635 |
| Vibrio | alginolyticus | 12G01 | 636-641 |
| Pseudoalteromonas | sp. | P1-11 | 642-650 |
| Pseudoalteromonas | sp. | P1-25 | 651-657 |
| Photobacterium | aquae | CGMCC 1.12159 | 658-667 |
| Photobacterium | ganghwense | DSM 22954 | 668-674 |
| Vibrio | vulnificus | VA-WGS-18042 | 675-680 |
| Vibrio | cholerae | P7-CHT61-04 | 681-687 |
| Vibrio | cholerae | A12JL36W17 | 688-693 |
| Pseudoalteromonas | sp. | — | 694-701 |
| Vibrio | parahaemolyticus | CFSAN007440 | 702-707 |
| Vibrio | navarrensis | ATCC 51183 | 708-715 |
| Vibrio | parahaemolyticus | 9.5357 | 716-721 |
| Vibrio | parahaemolyticus | 856038 | 722-727 |
| Vibrio | parahaemolyticus | MAVP-E | 728-735 |
| Vibrio | toranzoniae | Vb 10.8 | 736-739 |
| Vibrio | parahaemolyticus | 926501 | 740-747 |
| Shewanella | sp. | UCD-FRSSP16_17 | 748-754 |
| Vibrio | parahaemolyticus | GCSL_R146 | 755-760 |
| Halomonas | sp. | Soap Lake #7 | 761-768 |
| Vibrio | parahaemolyticus | MAVP-Q | 769-776 |
| Aeromonas | salmonicida | S44 | 777-781 |
| Vibrio | parahaemolyticus | G6928 | 782-789 |
| Vibrio | parahaemolyticus | MAVP-R | 790-797 |
| Vibrio | cholerae | OYP8C06 | 798-803 |
| Vibrio | cholerae | OYP8F12 | 804-809 |
| Vibrio | sp. | dhg | 810-815 |
| Vibrio | cholerae | N2808 | 816-822 |
| Vibrio | parahaemolyticus | CFSAN018753 | 823-827 |
| Vibrio | parahaemolyticus | CDC_K5073 | 828-833 |
| Pseudoalteromonas | issachenkonii | KCTC 12958 | 834-839 |
| Vibrio | natriegens | ATCC 14048 | 255-839 |
| Vibrio | fluvialis | NCTC11327 | 840-843 |
| Vibrio | parahaemolyticus | VP2007-095 | 844-851 |
| Aliivibrio | fischeri | ZF-211 | 852-853 |
| Vibrio | cholerae | YB4H02 | 854-859 |
| Vibrio | cholerae | OYP2C05 | 860-866 |
| Vibrio | cholerae | A110926W4 | 867-875 |
| Vibrio | vulnificus | BAA87 | 876-881 |
| Vibrio | splendidus | ZS_82 | 882-887 |
| Photobacterium | sanguinicancri | ME15 | 888-893 |
| Vibrio | cholerae | Drakes2013 | 894-900 |
| Shewanella | baltica | OS223 | 901-905 |
| Vibrio | sp. | 16 | 906-911 |
| Vibrio | sp. | HI00D65 | 912-917 |
| Vibrio | parahaemolyticus | CFSAN018752 | 918-922 |
| Vibrio | breoganii | 10N.286.49.E9 | 923-929 |
| Vibrio | lentus | 10N.286.51.C2 | 930-935 |
| Vibrio | sp. | 10N.261.46.E8 | 936-943 |
| Vibrio | crassostreae | 31_O_69 | 944-948 |
| Pseudoalteromonas | sp. | S1688 | 949-955 |
| Vibrio | parahaemolyticus | CFSAN007439 | 956-961 |
| Vibrio | parahaemolyticus | CFSAN007432 | 962-969 |
| Vibrio | parahaemolyticus | CFSAN007429 | 970-977 |
| Vibrio | cidicii | 2423-01 | 978-986 |
| Pseudoalteromonas | luteoviolacea | NCIMB 1942 | 987-995 |
| Aliivibrio | fischeri | 5F33 | 996-998 |
| Vibrio | parahaemolyticus | GCSL_R145 | 999-1004 |
| Vibrio | vulnificus | DAL-79087 | 1005-1012 |
| Vibrio | parahaemolyticus | MAVP-P | 1013-1020 |
| Vibrio | parahaemolyticus | MAVP-L | 1021-1028 |
| Vibrio | parahaemolyticus | MAVP30 | 1029-1036 |
| Vibrio | parahaemolyticus | MAVP75 | 1037-1044 |
| Vibrio | parahaemolyticus | MA561 | 1045-1052 |
| Vibrio | parahaemolyticus | MAVP-94 | 1053-1060 |
| Pseudoalteromonas | sp. | A757 | 1061-1070 |
| Vibrio | toranzoniae | CECT 7225 | 1071-1074 |
| Vibrio | cholerae | A12JL4W81 | 1075-1080 |
| Vibrio | vulnificus | VV9-09 | 1081-1086 |
| Vibrionales | bacterium | C3R12 | 1087-1092 |
| Vibrio | crassostreae | 43_P_280 | 1093-1098 |
| Vibrio | kanaloae | 10N.286.45.A9 | 1099-1104 |
| Enterovibrio | coralii | CAIM 912 | 1105-1111 |
| Vibrio | sp. | 2521-89 | 1112-1118 |
| Vibrio | diazotrophicus | 65.10M | 1119-1121 |
| Vibrio | vulnificus | 95-8-161 | 1122-1128 |
| Aeromonas | veronii | ML09-123 | 1129-1134 |
| Vibrio | vulnificus | VA-WGS-18036 | 1135-1140 |
| Vibrio | parahaemolyticus | B6_4 | 1141-1148 |
| Vibrio | sinaloensis | AD048 | 1149-1154 |
| Vibrio | vulnificus | Vv002 | 1155-1160 |
| Vibrio | alginolyticus | UCD-30C | 1161-1166 |
| Vibrio | crassostreae | J2-9 | 1167-1171 |
| Vibrio | splendidus | 10N.286.45.A10 | 1172-1177 |
| Vibrio | campbellii | ABR2-13 | 1178-1185 |
| Photobacterium | damselae | NCTC11648 | 1186-1192 |
| Vibrio | anguillarum | DSM 21597 | 1193-1198 |
| Vibrio | cyclitrophicus | 10N.286.54.C7 | 1199-1204 |
| Vibrio | lentus | 10N.286.49.C5 | 1205-1210 |
| Vibrio | lentus | 10N.261.48.B11 | 1211-1216 |
| Vibrio | diazotrophicus | 60.6F | 1217-1220 |
| Vibrio | crassostreae | LGP107 | 1221-1226 |
| Pseudoalteromonas | translucida | KMM 520 | 1227-1232 |
| Vibrio | coralliilyticus | AIC-7 | 1233-1240 |
| Vibrio | tasmaniensis | 10N.261.52.A6 | 1241-1246 |
| Vibrio | lentus | 10N.261.52.C5 | 1247-1252 |
| Vibrio | crassostreae | 26_O_11 | 1253-1258 |
| Vibrio | crassostreae | 38_P_219 | 1259-1264 |
| Vibrio | crassostreae | 30_P_64 | 1265-1269 |
| Shewanella | sp. | ANA-3 | 1270-1277 |
| Vibrio | sp. | 10N.286.45.B6 | 1278-1285 |
| Pseudoalteromonas | arctica | A 37-1-2 | 1286-1293 |
| Vibrio | natriegens | ATCC 14048 | 255-1293 |
| Photobacterium | kishitanii | GCSL-P50 | 1294-1302 |
| Pseudoalteromonas | arctica | MelAa3 | 317-322 |
| | | | and |
| | | | 1303-1308 |
| Pseudoalteromonas | sp. | GutCa3 | 323-328 |
| | | | and |
| | | | 1743-1748 |
| Vibrio | splendidus | 10N.222.46.B1 | 1309-1316 |
| Vibrio | breoganii | 10N.222.51.B10 | 1317-1321 |
| Enterovibrio | norvegicus | 10N.261.45.A10 | 1322-1329 |
| Photobacterium | kishitanii | DSMZ 2167 | 1330-1338 |
| Photobacterium | kishitanii | calba.1.1 | 1339-1347 |
| Vibrio | splendidus | ZS_185 | 1348-1353 |
| Vibrio | breoganii | 10N.222.46.E5 | 1354-1359 |
| Pseudoalteromonas | sp. | S4498 | 1360-1365 |
| Vibrio | crassostreae | J5-15 | 1366-1371 |
| Photobacterium | leiognathi | Res.4.1 | 1372-1381 |
| Vibrio | cholerae | YB4G06 | 1382-1387 |
| Vibrio | lentus | 10N.261.45.C3 | 1388-1393 |
| Pseudoalteromonas | sp. | S4389 | 1394-1401 |
| Photobacterium | sp. | BEI 247 | 1402-1407 |
| Enterovibrio | sp. | CAIM 600 | 1408-1417 |
| Vibrio | parahaemolyticus | CDC-AM50933 | 1418-1423 |
| Shewanella | putrefaciens | NCTC10695 | 1424-1430 |
| Vibrio | azureus | NBRC 104587 | 1431-1437 |
| Vibrio | parahaemolyticus | A4EZ703 | 1438-1443 |
| Photobacterium | proteolyticum | 13-12 | 1444-1449 |
| Vibrio | cholerae | OYPIG01 | 1450-1457 |
| Pseudoalteromonas | sp. | SW0106-04 | 1458-1467 |
| Parashewanella | spongiae | KCTC 22492 | 1468-1476 |
| Photobacterium | angustum | S14 | 1477-1483 |
| Vibrio | breoganii | ZF-29 | 1484-1489 |
| Vibrio | cholerae | 09_113 | 1490-1496 |
| Pseudoalteromonas | sp. | SCSIO_11900 | 1497-1504 |
| Aliivibrio | fischeri | MJ11 | 1505-1507 |
| Vibrionales | bacterium | SWAT-3 | 1508-1513 |
| Pseudoalteromonas | sp. | BSi20495 | 1514-1520 |
| Vibrio | parahaemolyticus | CFSAN007436 | 1521-1528 |
| Vibrio | parahaemolyticus | CFSAN007431 | 1529-1536 |
| Vibrio | parahaemolyticus | CFSAN007430 | 1537-1544 |
| Vibrio | parahaemolyticus | CFSAN007433 | 1545-1552 |
| Vibrio | parahaemolyticus | CFSAN007434 | 1553-1560 |
| Vibrio | vulnificus | 491771 | 1561-1566 |
| Vibrio | diabolicus | V2 | 1567-1572 |
| Vibrio | sp. | J2-3 | 1573-1578 |
| Vibrio | parahaemolyticus | S487-4 | 1579-1586 |
| Vibrio | cholerae | YB2G01 | 1587-1592 |
| Vibrio | harveyi | FDAARGOS_106 | 1593-1600 |
| Vibrio | anguillarum | 90-11-286 | 1601-1606 |
| Vibrio | parahaemolyticus | — | 1607-1614 |
| Aliivibrio | sp. | 1S175 | 1615-1616 |
| Vibrio | parahaemolyticus | KVp10 | 1617-1622 |
| Vibrio | parahaemolyticus | CDC_K4762 | 1623-1629 |
| Vibrio | parahaemolyticus | CDC_K5582 | 1630-1637 |
| Vibrio | mimicus | SCCF01 | 1638-1643 |
| Oceanospirillum | linum | ATCC 11336 | 1644-1658 |
| Vibrio | campbellii | ATCC 25920, | 1659-1664 |
| | | CAIM 519T | |
| Vibrio | parahaemolyticus | MAVP-A | 1665-1672 |
| Vibrio | parahaemolyticus | MAVP-T | 1673-1680 |
| Vibrio | parahaemolyticus | MAVP39 | 1681-1688 |
| Vibrio | parahaemolyticus | MAVP74 | 1689-1696 |
| Vibrio | parahaemolyticus | G149 | 1697-1704 |
| Vibrio | parahaemolyticus | MAVP-109 | 1705-1712 |
| Vibrio | parahaemolyticus | MEVP12 | 1713-1720 |
| Vibrio | parahaemolyticus | CTVP31C | 1721-1728 |
| Vibrio | parahaemolyticus | CTVP27C | 1729-1736 |
| Pseudoalteromonas | issachenkonii | KMM 3549 | 1737-1742 |
| Pseudoalteromonas | sp. | GutCa3 | 323-328 |
| | | | and |
| | | | 1743-1748 |
| Vibrio | lentus | 10N.286.45.C8 | 1749-1754 |
| Vibrio | lentus | 10N.286.51.B9 | 1755-1760 |
| Vibrio | breoganii | 10N.222.49.A5 | 1761-1765 |
| Vibrio | sp. | 10N.261.46.F12 | 1766-1773 |
| Vibrio | sp. | 10N.261.49.E11 | 1774-1781 |
| Photobacterium | damselae | 940804-1/1 | 1782-1787 |
| Photobacterium | ganghwense | JCM 12487 | 1788-1797 |
| Vibrio | splendidus | ZS_173 | 1798-1803 |
| Vibrio | cholerae | 3523-03 | 1804-1811 |
| Vibrio | cholerae | 20000 | 1812-1818 |
| Vibrio | crassostreae | 29_O_38 | 1819-1824 |
| Vibrio | crassostreae | 16BF1_56 | 1825-1830 |
| Psychromonas | sp. | RZ5 | 1831-1836 |
| Pseudoalteromonas | sp. | S983 | 1837-1843 |
| Vibrio | parahaemolyticus | E1_5 | 1844-1849 |
| Pseudoalteromonas | luteoviolacea | H2 | 1850-1855 |
| Vibrio | cholerae | A110621W3 | 1856-1863 |
| Neptunomonas | qingdaonensis | CGMCC 1.10971 | 1864-1871 |

CRISPR I—F3 system elements, e.g. proteins or nucleic acid sequences encoding such proteins may be derived from any one of the organisms as set forth in Table A or Table B. In some embodiments, the I—F3 system is a I-F3b system and the proteins or elements of the I-F3b system are derived or obtained from an organism in Table A. Organisms that are listed in both Table A and Table B may be excluded from the Table B list, to the extent they express a non-I-F3b system that may function only with conventional guide RNAs. In general, it is considered, and as further described below, that I-F3a systems primarily use attachment sites adjacent to the yciA and guaC (IMPDH) genes. I-F3b elements are primarily found in an attachment site downstream of the ffs gene encoding the RNA component of the signal recognition particle and a minor branch with elements residing downstream of the rsmJ gene.

TABLE B
Organisms with IF-3a systems.

| | | | SEQ ID |
|---|---|---|---|
| Genus | species | strain | NOS |
| Vibrio | parahaemolyticus | CFSAN007436 | 1872-1878 |
| Vibrio | parahaemolyticus | CFSAN007462 | 1879-1885 |
| Vibrio | parahaemolyticus | CFSAN006131 | 1886-1892 |
| Vibrio | parahaemolyticus | CFSAN007458 | 1893-1899 |
| Vibrio | parahaemolyticus | CFSAN007435 | 1900-1906 |
| Vibrio | parahaemolyticus | CFSAN007454 | 1907-1913 |
| Vibrio | parahaemolyticus | EN9701173 | 1914-1920 |
| Vibrio | parahaemolyticus | EN9901310 | 1921-1927 |
| Vibrio | parahaemolyticus | 856769 | 1928-1934 |
| Vibrio | parahaemolyticus | 867361 | 1935-1941 |
| Vibrio | parahaemolyticus | 858960 | 1942-1949 |
| Vibrio | parahaemolyticus | HS-13-1 | 1950-1956 |
| Vibrio | parahaemolyticus | S499-7 | 1957-1963 |
| Vibrio | parahaemolyticus | FDAARGOS_120 | 1964-1970 |
| Vibrio | parahaemolyticus | A4EZ927 | 1971-1978 |
| Vibrio | parahaemolyticus | A4EZ724 | 1979-1985 |
| Vibrio | parahaemolyticus | A2EZ614 | 1986-1992 |
| Vibrio | parahaemolyticus | A2EZ743 | 1993-1999 |
| Vibrio | parahaemolyticus | A2EZ523 | 2000-2006 |
| Vibrio | parahaemolyticus | A3EZ634 | 2007-2013 |
| Vibrio | parahaemolyticus | 106744 | 2014-2021 |
| Vibrio | parahaemolyticus | A5Z273 | 2022-2029 |
| Vibrio | parahaemolyticus | CFSAN025067 | 2030-2038 |
| Vibrio | parahaemolyticus | CFSAN025068 | 2039-2047 |
| Vibrio | parahaemolyticus | CFSAN025058 | 2048-2056 |
| Vibrio | parahaemolyticus | CFSAN025061 | 2057-2065 |
| Vibrio | parahaemolyticus | GCSL_R26 | 2066-2072 |
| Vibrio | parahaemolyticus | GCSL_R75 | 2073-2079 |
| Vibrio | parahaemolyticus | GCSL_R145 | 2080-2086 |
| Vibrio | parahaemolyticus | CDC_K5428 | 2087-2093 |
| Vibrio | parahaemolyticus | CDC_K5328 | 2094-2100 |
| Vibrio | parahaemolyticus | CDC_K5010G | 2101-2108 |
| Vibrio | parahaemolyticus | CDC_K5010W | 2109-2116 |
| Vibrio | parahaemolyticus | CDC_K5439 | 2117-2124 |
| Vibrio | parahaemolyticus | CDC_K5457 | 2125-2131 |
| Vibrio | parahaemolyticus | CAIM 1772 | 2132-2138 |
| Vibrio | parahaemolyticus | PMA11.14 | 2139-2146 |
| Vibrio | parahaemolyticus | PMA32.14 | 2147-2154 |
| Vibrio | parahaemolyticus | 09-3216_1 | 2155-2161 |
| Vibrio | parahaemolyticus | HS-13-1_1 | 2162-2168 |
| Vibrio | parahaemolyticus | CFSAN001598 | 2169-2175 |
| Vibrio | parahaemolyticus | CFSAN018768 | 2176-2182 |
| Vibrio | parahaemolyticus | CFSAN018774 | 2183-2189 |
| Vibrio | parahaemolyticus | CFSAN018777 | 2190-2196 |
| Vibrio | parahaemolyticus | MAVP4 | 2197-2203 |
| Vibrio | parahaemolyticus | MAVP78 | 2204-2210 |
| Vibrio | parahaemolyticus | MAVP-94 | 2211-2217 |
| Vibrio | parahaemolyticus | MEVP12 | 2218-2224 |
| Vibrio | anguillarum | VIB43 | 2225-2227 |
| Vibrio | diazotrophicus | 60.18M | 2228-2236 |
| Vibrio | diazotrophicus | 65.7M | 2237-2243 |
| Photobacterium | iliopiscarium | NCIMB 13478 | 2244-2252 |
| Photobacterium | iliopiscarium | NCIMB 13355 | 2253-2266 |
| Vibrio | parahaemolyticus | FORC_071 | 2267-2274 |
| Vibrio | parahaemolyticus | Vp46 | 2275-2281 |
| Vibrio | parahaemolyticus | G1_3 | 2282-2289 |
| Vibrio | parahaemolyticus | S175 | 2290-2297 |
| Vibrio | parahaemolyticus | F3_1 | 2298-2305 |
| Vibrio | parahaemolyticus | B4_5 | 2306-2312 |
| Vibrio | parahaemolyticus | VP130064 | 2313-2320 |
| Vibrio | parahaemolyticus | VP161109 | 2321-2329 |
| Vibrio | parahaemolyticus | VP161504 | 2330-2337 |
| Vibrio | parahaemolyticus | VP150615 | 2338-2345 |
| Vibrio | parahaemolyticus | HZ17-083 | 2346-2353 |
| Vibrio | parahaemolyticus | HZ17-018 | 2354-2360 |
| Vibrio | parahaemolyticus | VPCR-2010 | 2361-2368 |
| Vibrio | parahaemolyticus | SBR10290 | 2369-2375 |
| Vibrio | parahaemolyticus | CFSAN007443 | 2376-2386 |
| Vibrio | parahaemolyticus | CFSAN007445 | 2387-2393 |
| Vibrio | parahaemolyticus | CFSAN007430 | 2394-2400 |
| Vibrio | parahaemolyticus | CFSAN007432 | 2401-2407 |
| Vibrio | parahaemolyticus | CFSAN001613 | 2408-2414 |
| Vibrio | parahaemolyticus | CFSAN007449 | 2415-2422 |
| Vibrio | parahaemolyticus | 846 | 2423-2429 |
| Vibrio | parahaemolyticus | VP551 | 2430-2437 |
| Vibrio | parahaemolyticus | 877952 | 2438-2444 |
| Vibrio | parahaemolyticus | 1934965 | 2445-2451 |
| Vibrio | parahaemolyticus | 389167 | 2452-2460 |
| Vibrio | parahaemolyticus | 08-0278 | 2461-2468 |
| Vibrio | parahaemolyticus | MAVP-V | 2469-2475 |
| Vibrio | parahaemolyticus | PMA37.5 | 2476-2483 |
| Vibrio | parahaemolyticus | ATC210 | 2484-2491 |
| Vibrio | fluvialis | FDAARGOS_104 | 2492-2499 |
| Vibrio | parahaemolyticus | H11523 | 2500-2506 |
| Vibrio | parahaemolyticus | A0EZ664 | 2507-2513 |
| Vibrio | parahaemolyticus | F1419 | 2514-2520 |
| Vibrio | parahaemolyticus | 482000 | 2521-2527 |
| Vibrio | parahaemolyticus | C144 | 2528-2534 |
| Vibrio | parahaemolyticus | 1056404 | 2535-2541 |
| Vibrio | parahaemolyticus | A4EZ964 | 2542-2548 |
| Vibrio | parahaemolyticus | 237500 | 2549-2555 |
| Vibrio | parahaemolyticus | CFSAN029656 | 2556-2564 |
| Vibrio | parahaemolyticus | CFSAN025054 | 2565-2573 |
| Vibrio | parahaemolyticus | CFSAN001174 | 2574-2580 |
| Vibrio | parahaemolyticus | CFSAN018763 | 2581-2587 |
| Vibrio | parahaemolyticus | GCSL_R47 | 2588-2594 |
| Vibrio | parahaemolyticus | GCSL_R51 | 2595-2601 |
| Vibrio | parahaemolyticus | GCSL_R131 | 2602-2608 |
| Vibrio | parahaemolyticus | GCSL_R136 | 2609-2615 |
| Vibrio | parahaemolyticus | CDC_K5324W | 2616-2622 |
| Vibrio | parahaemolyticus | CDC_K5308 | 2623-2629 |
| Vibrio | parahaemolyticus | CDC_K4639W | 2630-2636 |
| Vibrio | parahaemolyticus | CDC_K4639G | 2637-2643 |
| Vibrio | parahaemolyticus | CDC_K5276 | 2644-2650 |
| Vibrio | parahaemolyticus | CDC_K5582 | 2651-2657 |
| Vibrio | parahaemolyticus | CDC_K5618 | 2658-2664 |
| Vibrio | parahaemolyticus | GCSL_R130 | 2665-2671 |
| Vibrio | parahaemolyticus | CFSAN026729 | 2672-2678 |
| Vibrio | parahaemolyticus | CFSAN001615 | 2679-2685 |
| Vibrio | parahaemolyticus | CFSAN001604 | 2686-2692 |
| Vibrio | parahaemolyticus | CFSAN018769 | 2693-2699 |
| Vibrio | parahaemolyticus | CFSAN018775 | 2700-2706 |
| Vibrio | parahaemolyticus | MAVP-A | 2707-2713 |
| Vibrio | parahaemolyticus | MAVP-P | 2714-2720 |
| Vibrio | parahaemolyticus | MAVP30 | 2721-2727 |
| Vibrio | parahaemolyticus | MAVP-90 | 2728-2734 |
| Vibrio | parahaemolyticus | MAVP-50 | 2735-2741 |
| Vibrio | parahaemolyticus | CDC-AM43539 | 2742-2748 |
| Vibrio | lentus | 10N.286.46.B8 | 2749-2756 |
| Vibrio | cyclitrophicus | 10N.261.55.A11 | 2757-2759 |
| Photobacterium | sp. | GB-72 | 2760-2766 |
| Photobacterium | leiognathi | ATCC 25521 | 2767-2775 |
| Vibrio | sp. | 10N.222.48.A9 | 2776-2778 |
| Vibrio | parahaemolyticus | G1_6 | 2779-2786 |
| Vibrio | parahaemolyticus | F3_7 | 2787-2794 |
| Vibrio | parahaemolyticus | F2_9 | 2795-2802 |
| Vibrio | parahaemolyticus | E3_10 | 2803-2809 |
| Vibrio | parahaemolyticus | D7_4 | 2810-2816 |
| Vibrio | parahaemolyticus | VP090004 | 2817-2824 |
| Vibrio | parahaemolyticus | VP170075 | 2825-2832 |
| Vibrio | parahaemolyticus | VP161603 | 2833-2840 |
| Vibrio | parahaemolyticus | VP162180 | 2841-2848 |
| Vibrio | parahaemolyticus | HZ13-088 | 2849-2856 |
| Vibrio | parahaemolyticus | HZ02-J7 | 2857-2864 |
| Vibrio | parahaemolyticus | AQ3810 | 2865-2872 |
| Vibrio | parahaemolyticus | 10290 | 2873-2879 |
| Vibrio | parahaemolyticus | 50 | 2880-2886 |
| Vibrio | parahaemolyticus | S089 | 2887-2894 |
| Vibrio | parahaemolyticus | F3_4 | 2895-2902 |
| Vibrio | parahaemolyticus | F2_7 | 2903-2910 |
| Vibrio | parahaemolyticus | C2_1 | 2911-2917 |
| Vibrio | parahaemolyticus | VP080007 | 2918-2925 |
| Vibrio | parahaemolyticus | VP140095 | 2926-2933 |
| Vibrio | parahaemolyticus | VP160744 | 2934-2941 |
| Vibrio | parahaemolyticus | VP161602 | 2942-2949 |
| Vibrio | parahaemolyticus | HZ16-338 | 2950-2957 |
| Vibrio | parahaemolyticus | EKP-021 | 2958-2965 |
| Vibrio | parahaemolyticus | VP161904 | 2966-2973 |
| Vibrio | campbellii | ABR2-13 | 2974-2981 |
| Vibrio | rotiferianus | CAIM 577 | 2982-2988 |
| Vibrio | cyclitrophicus | 10N.286.55.F1 | 2989-2995 |
| Photobacterium | leiognathi | PL-721 | 2996-3003 |
| Vibrio | parahaemolyticus | S176 | 3004-3010 |
| Vibrio | parahaemolyticus | AQ3810 | 2865-2872 |
| Vibrio | cholerae | FORC_076 | 3011-3020 |
| Vibrio | parahaemolyticus | O3:K6 substr. RIMD | 3021-3028 |
| | | 2210633 | |
| Vibrio | parahaemolyticus | CFSAN001617 | 3029-3035 |
| Vibrio | parahaemolyticus | CFSAN007456 | 3036-3042 |
| Vibrio | parahaemolyticus | CFSAN007429 | 3043-3049 |
| Vibrio | parahaemolyticus | CFSAN007433 | 3050-3056 |
| Vibrio | parahaemolyticus | CFSAN001620 | 3057-3063 |
| Vibrio | parahaemolyticus | CFSAN006132 | 3064-3070 |
| Vibrio | parahaemolyticus | 863 | 3071-3078 |
| Vibrio | parahaemolyticus | EN9701121 | 3079-3085 |
| Vibrio | parahaemolyticus | 857499 | 3086-3092 |
| Vibrio | parahaemolyticus | 855308 | 3093-3099 |
| Vibrio | parahaemolyticus | 04-1290 | 3100-3106 |
| Vibrio | parahaemolyticus | 07-1339 | 3107-3114 |
| Vibrio | parahaemolyticus | FDAARGOS_52 | 3115-3121 |
| Vibrio | parahaemolyticus | S383-6 | 3122-3128 |
| Vibrio | parahaemolyticus | S440-7 | 3129-3135 |
| Vibrio | parahaemolyticus | ATC220 | 3136-3143 |
| Vibrio | parahaemolyticus | S372-5 | 3144-3150 |
| Vibrio | parahaemolyticus | A4EZ700 | 3151-3157 |
| Vibrio | parahaemolyticus | A1EZ919 | 3158-3164 |
| Vibrio | parahaemolyticus | 926501 | 3165-3171 |
| Vibrio | parahaemolyticus | A2EZ715 | 3172-3178 |
| Vibrio | parahaemolyticus | F63267 | 3179-3186 |
| Vibrio | parahaemolyticus | A3EZ710 | 3187-3193 |
| Vibrio | parahaemolyticus | A3EZ936 | 3194-3200 |
| Vibrio | parahaemolyticus | C147 | 3201-3207 |
| Vibrio | parahaemolyticus | C140 | 3208-3216 |
| Vibrio | parahaemolyticus | A5Z924 | 3217-3223 |
| Vibrio | parahaemolyticus | CFSAN025065 | 3224-3232 |
| Vibrio | parahaemolyticus | CFSAN025059 | 3233-3241 |
| Vibrio | parahaemolyticus | CFSAN025053 | 3242-3250 |
| Vibrio | anguillarum | 90-11-286 | 3251-3257 |
| Vibrio | parahaemolyticus | — | 3258-3264 |
| Vibrio | parahaemolyticus | Klin | 3265-3272 |
| Vibrio | parahaemolyticus | GCSL_R31 | 3273-3279 |
| Vibrio | parahaemolyticus | GCSL_R146 | 3280-3286 |
| Vibrio | parahaemolyticus | GCSL_R150 | 3287-3293 |
| Vibrio | parahaemolyticus | CDC_K5281 | 3294-3300 |
| Vibrio | parahaemolyticus | CDC_K4764D | 3301-3307 |
| Vibrio | parahaemolyticus | CDC_K5331 | 3308-3315 |
| Vibrio | parahaemolyticus | CDC_K5512 | 3316-3322 |
| Vibrio | parahaemolyticus | CDC_K5638 | 3323-3329 |
| Vibrio | parahaemolyticus | GIMxtfL61-2011.05 | 3330-3337 |
| Vibrio | parahaemolyticus | GIMxtfL65-2011.05 | 3338-3345 |
| Vibrio | parahaemolyticus | PMC53.7 | 3346-3352 |
| Vibrio | parahaemolyticus | PMA31.14 | 3353-3360 |
| Vibrio | parahaemolyticus | CFSAN026730 | 3361-3367 |
| Vibrio | vulnificus | VN-0206 | 3368-3374 |
| Vibrio | parahaemolyticus | FDAARGOS_191 | 3375-3382 |
| Vibrio | parahaemolyticus | Vp230 | 3383-3385 |
| Vibrio | parahaemolyticus | CFSAN001605 | 3386-3392 |
| Vibrio | parahaemolyticus | CFSAN001597 | 3393-3399 |
| Vibrio | parahaemolyticus | CFSAN022334 | 3400-3406 |
| Vibrio | parahaemolyticus | CFSAN022335 | 3407-3413 |
| Vibrio | parahaemolyticus | MAVP-T | 3414-3420 |
| Vibrio | parahaemolyticus | MAVP74 | 3421-3427 |
| Vibrio | mediterranei | QT6D1 | 3428-3434 |
| Vibrio | parahaemolyticus | MAVP-71 | 3435-3441 |
| Vibrio | parahaemolyticus | CTVP31C | 3442-3448 |
| Vibrio | parahaemolyticus | L70 | 3449-3456 |
| Vibrio | mediterranei | 117-T6 | 3457-3463 |
| Vibrio | cholerae | N2822 | 3464-3472 |
| Vibrio | parahaemolyticus | MAVP-109 | 3473-3479 |
| Vibrio | parahaemolyticus | CFSAN018753 | 3480-3486 |
| Vibrio | parahaemolyticus | GCSL_R87 | 3487-3493 |
| Vibrio | parahaemolyticus | GCSL_R88 | 3494-3500 |
| Vibrio | parahaemolyticus | CDC_K4636 | 3501-3507 |
| Vibrio | parahaemolyticus | CDC_K4637G | 3508-3515 |
| Vibrio | parahaemolyticus | CDC_K5073 | 3516-3522 |
| Vibrio | parahaemolyticus | 3259 | 3523-3530 |
| Vibrio | parahaemolyticus | 12310 | 3531-3537 |
| Vibrio | parahaemolyticus | EKP-008 | 3538-3545 |
| Vibrio | parahaemolyticus | CFSAN001612 | 3546-3552 |
| Vibrio | fluvialis | NCTC11327 | 3553-3560 |
| Vibrio | parahaemolyticus | EKP-028 | 3561-3568 |
| Vibrio | parahaemolyticus | EKP-026 | 3569-3576 |
| Vibrio | parahaemolyticus | HS-06-05 | 3577-3583 |
| Vibrio | parahaemolyticus | MAVP-112 | 3584-3590 |
| Vibrio | parahaemolyticus | Vp5 | 3591-3597 |
| Vibrio | alginolyticus | V1 | 3598-3604 |
| Vibrio | parahaemolyticus | CT4287 | 3605-3611 |
| Vibrio | anguillarum | JLL237 | 3612-3618 |
| Vibrio | parahaemolyticus | VP170801 | 3619-3626 |
| Vibrio | parahaemolyticus | Gxw_9143 | 3627-3634 |
| Vibrio | parahaemolyticus | CFSAN018752 | 3635-3641 |
| Vibrio | owensii | 1700302 | 3642-3648 |
| Photobacterium | piscicola | type strain: NCCB | 3649-3656 |
| | | 100098 | |
| Vibrio | parahaemolyticus | CFSAN025055 | 3657-3665 |
| Vibrio | parahaemolyticus | GCSL_R137 | 3666-3672 |
| Vibrio | parahaemolyticus | CFSAN001621 | 3673-3679 |
| Vibrio | parahaemolyticus | AN-5034 | 3680-3687 |
| Vibrio | parahaemolyticus | CFSAN006130 | 3688-3694 |
| Vibrio | parahaemolyticus | CFSAN007461 | 3695-3701 |
| Vibrio | parahaemolyticus | CFSAN007442 | 3702-3708 |
| Vibrio | parahaemolyticus | CFSAN007431 | 3709-3715 |
| Vibrio | parahaemolyticus | CFSAN007434 | 3716-3722 |
| Vibrio | parahaemolyticus | CFSAN001618 | 3723-3729 |
| Vibrio | parahaemolyticus | K1203 | 3730-3736 |
| Vibrio | parahaemolyticus | CFSAN007450 | 3737-3744 |
| Vibrio | parahaemolyticus | CFSAN006133 | 3745-3751 |
| Vibrio | parahaemolyticus | 97-10290 | 3752-3758 |
| Vibrio | parahaemolyticus | 3324 | 3759-3765 |
| Vibrio | parahaemolyticus | EN2910 | 3766-3772 |
| Vibrio | parahaemolyticus | 3644 | 3773-3779 |
| Vibrio | parahaemolyticus | 874301 | 3780-3786 |
| Vibrio | parahaemolyticus | 857865 | 3787-3793 |
| Vibrio | parahaemolyticus | 855673 | 3794-3800 |
| Vibrio | parahaemolyticus | 237135 | 3801-3808 |
| Vibrio | parahaemolyticus | 481270 | 3809-3815 |
| Vibrio | parahaemolyticus | 237865 | 3816-3823 |
| Vibrio | parahaemolyticus | 872109 | 3824-3830 |
| Vibrio | parahaemolyticus | MAVP-E | 3831-3837 |
| Vibrio | parahaemolyticus | FDAARGOS_51 | 3838-3844 |
| Vibrio | parahaemolyticus | S448-16 | 3845-3851 |
| Vibrio | parahaemolyticus | S487-4 | 3852-3858 |
| Vibrio | parahaemolyticus | PMC58.5 | 3859-3866 |
| Vibrio | parahaemolyticus | PMC14.7 | 3867-3874 |
| Vibrio | parahaemolyticus | K23 | 3875-3881 |
| Vibrio | parahaemolyticus | M59787 | 3882-3888 |
| Vibrio | parahaemolyticus | H64024 | 3889-3895 |
| Vibrio | parahaemolyticus | F4395 | 3896-3902 |
| Vibrio | parahaemolyticus | A3EZ136 | 3903-3910 |
| Vibrio | parahaemolyticus | T8994 | 3911-3917 |
| Vibrio | parahaemolyticus | 388802 | 3918-3925 |
| Vibrio | parahaemolyticus | A0EZ383 | 3926-3933 |
| Vibrio | parahaemolyticus | A3EZ770 | 3934-3940 |
| Vibrio | parahaemolyticus | A1EZ952 | 3941-3947 |
| Vibrio | parahaemolyticus | C146 | 3948-3954 |
| Vibrio | parahaemolyticus | A5Z652 | 3955-3961 |
| Vibrio | parahaemolyticus | A5Z860 | 3962-3968 |
| Vibrio | parahaemolyticus | A5Z878 | 3969-3975 |
| Vibrio | parahaemolyticus | CFSAN025063 | 3976-3984 |
| Vibrio | parahaemolyticus | CFSAN029653 | 3985-3993 |
| Vibrio | parahaemolyticus | CFSAN025064 | 3994-4002 |
| Vibrio | parahaemolyticus | CFSAN025056 | 4003-4011 |
| Vibrio | parahaemolyticus | CFSAN018767 | 4012-4018 |
| Vibrio | parahaemolyticus | GCSL_R10 | 4019-4025 |
| Vibrio | parahaemolyticus | GCSL_R16 | 4026-4032 |
| Vibrio | parahaemolyticus | GCSL_R33 | 4033-4043 |
| Vibrio | parahaemolyticus | GCSL_R76 | 4044-4050 |
| Vibrio | parahaemolyticus | GCSL_R144 | 4051-4057 |
| Vibrio | parahaemolyticus | CDC_K5433 | 4058-4064 |
| Vibrio | parahaemolyticus | CDC_K5306 | 4065-4071 |
| Vibrio | parahaemolyticus | CDC_K5429 | 4072-4078 |
| Vibrio | parahaemolyticus | CDC_K4558G | 4079-4081 |
| Vibrio | parahaemolyticus | CDC_K4763 | 4082-4088 |
| Vibrio | parahaemolyticus | CDC_K4775 | 4089-4096 |
| Vibrio | parahaemolyticus | CDC_K5058 | 4097-4104 |
| Vibrio | parahaemolyticus | CDC_K5278 | 4105-4111 |
| Vibrio | parahaemolyticus | CDC_K5345G | 4112-4118 |
| Vibrio | parahaemolyticus | CDC_K5456 | 4119-4125 |
| Vibrio | parahaemolyticus | CDC_K5528 | 4126-4133 |
| Vibrio | parahaemolyticus | CDC_K5620 | 4134-4140 |
| Vibrio | cholerae | VC22 | 4141-4143 |
| Vibrio | parahaemolyticus | GIMxtfL71-2011.05 | 4144-4150 |
| Vibrio | parahaemolyticus | PMA21.14 | 4151-4158 |
| Vibrio | parahaemolyticus | CFSAN001616 | 4159-4165 |
| Vibrio | parahaemolyticus | HS-13-1_100 | 4166-4172 |
| Vibrio | parahaemolyticus | Vp47 | 4173-4179 |
| Vibrio | parahaemolyticus | CFSAN001603 | 4180-4186 |
| Vibrio | parahaemolyticus | CFSAN001608 | 4187-4193 |
| Vibrio | parahaemolyticus | CFSAN001602 | 4194-4200 |
| Vibrio | parahaemolyticus | CFSAN001600 | 4201-4207 |
| Vibrio | parahaemolyticus | CFSAN018770 | 4208-4214 |
| Vibrio | parahaemolyticus | CFSAN022330 | 4215-4221 |
| Vibrio | parahaemolyticus | CFSAN022331 | 4222-4228 |
| Vibrio | parahaemolyticus | CFSAN022336 | 4229-4235 |
| Vibrio | parahaemolyticus | MAVP39 | 4236-4242 |
| Vibrio | parahaemolyticus | MAVP-Q | 4243-4249 |
| Vibrio | parahaemolyticus | G3578 | 4250-4256 |
| Vibrio | parahaemolyticus | MEVP14 | 4257-4263 |
| Vibrio | parahaemolyticus | CTVP34C | 4264-4270 |
| Vibrio | parahaemolyticus | CTVP27C | 4271-4277 |
| Vibrio | parahaemolyticus | VPD14 | 4278-4285 |
| Vibrio | parahaemolyticus | B-265 | 4286-4293 |
| Vibrio | parahaemolyticus | GCSL_R125 | 4294-4300 |
| Vibrio | parahaemolyticus | CDC_K4637W | 4301-4308 |
| Vibrio | cholerae | N2797 | 4309-4317 |
| Vibrio | parahaemolyticus | CDC_K5125 | 4318-4325 |
| Vibrio | parahaemolyticus | CFSAN001595 | 4326-4333 |
| Vibrio | parahaemolyticus | 09-3216_100 | 4334-4340 |
| Vibrio | natriegens | CCUG 16374 | 4341-4350 |
| Vibrio | parahaemolyticus | HZ18-100 | 4351-4359 |
| Vibrio | parahaemolyticus | CFSAN006135 | 4360-4366 |
| Vibrio | parahaemolyticus | ISF-01-07 | 4367-4374 |
| Vibrio | parahaemolyticus | ISF-25-6 | 4375-4382 |
| Vibrio | parahaemolyticus | CFSAN018757 | 4383-4390 |
| Vibrio | parahaemolyticus | PMA37.5 | 2476-2483 |
| Vibrio | parahaemolyticus | MAVP-14 | 4391-4397 |
| Vibrio | diabolicus | FDAARGOS_105 | 4398-4404 |
| Vibrio | parahaemolyticus | S107 | 4405-4412 |
| Vibrio | parahaemolyticus | S061 | 4413-4420 |
| Vibrio | parahaemolyticus | G2_2 | 4421-4428 |
| Vibrio | parahaemolyticus | G1_10 | 4429-4436 |
| Vibrio | parahaemolyticus | F8_5 | 4437-4443 |
| Vibrio | parahaemolyticus | F3_10 | 4444-4451 |
| Vibrio | parahaemolyticus | F3_3 | 4452-4459 |
| Vibrio | parahaemolyticus | F3_8 | 4460-4467 |
| Vibrio | parahaemolyticus | F2_10 | 4468-4475 |
| Vibrio | parahaemolyticus | E1_9 | 4476-4483 |
| Vibrio | parahaemolyticus | VP120036 | 4484-4491 |
| Vibrio | parahaemolyticus | VP150244 | 4492-4499 |
| Vibrio | parahaemolyticus | VP100044 | 4500-4507 |
| Vibrio | parahaemolyticus | VP160675 | 4508-4516 |
| Vibrio | parahaemolyticus | VP160483 | 4517-4524 |
| Vibrio | parahaemolyticus | VP140355 | 4525-4532 |
| Vibrio | parahaemolyticus | VP161902 | 4533-4540 |
| Vibrio | parahaemolyticus | HZ16-515 | 4541-4548 |
| Vibrio | parahaemolyticus | HZ15-046 | 4549-4556 |
| Vibrio | parahaemolyticus | HZ14-002 | 4557-4565 |
| Vibrio | parahaemolyticus | HZ13-043 | 4566-4573 |
| Vibrio | cholerae | N2825 | 4574-4583 |
| Vibrio | parahaemolyticus | VP161168 | 4584-4591 |
| Vibrio | parahaemolyticus | FDAARGOS_53 | 4592-4598 |
| Vibrio | cyclitrophicus | ZF99 | 4599-4605 |
| Vibrio | tasmaniensis | 10N.261.51.E11 | 4606-4612 |
| Vibrio | parahaemolyticus | 3256 | 4613-4619 |
| Photobacterium | iliopiscarium | ATCC 51760 | 4620-4633 |
| Vibrio | sp. | PID23_8 | 4634-4642 |
| Vibrio | parahaemolyticus | NIHCB0603 | 4643-4650 |
| Vibrio | parahaemolyticus | 949 | 4651-4658 |
| Vibrio | parahaemolyticus | VP2007-095 | 4659-4665 |
| Vibrio | parahaemolyticus | VP232 | 4666-4673 |
| Vibrio | parahaemolyticus | Peru-288 | 4674-4681 |
| Vibrio | parahaemolyticus | 861 | 4682-4689 |
| Vibrio | parahaemolyticus | V14/01 | 4690-4697 |
| Vibrio | parahaemolyticus | V-223/04 | 4698-4707 |
| Vibrio | parahaemolyticus | 10296 | 4708-4714 |
| Vibrio | parahaemolyticus | NCTC10884 | 4715-4722 |
| Vibrio | parahaemolyticus | FDA_R31 | 4723-4729 |
| Vibrio | parahaemolyticus | IDH02640 | 4730-4737 |
| Vibrio | parahaemolyticus | VP-48 | 4738-4745 |
| Vibrio | parahaemolyticus | VPTS-2010 | 4746-4748 |
| Vibrio | hangzhouensis | CGMCC 1.7062 | 4749-4753 |
| Vibrio | parahaemolyticus | HZ18-062 | 4754-4761 |
| Vibrio | cholerae | HE-45 | 4762-4771 |
| Vibrio | sp. | LJC006 | 4772-4779 |
| Vibrio | sp. | 10N.286.46.E4 | 4780-4787 |
| Vibrio | parahaemolyticus | 605 | 4788-4795 |
| Vibrio | anguillarum | J360 | 4796-4798 |
| Vibrio | parahaemolyticus | Peru-466 | 4799-4806 |
| Vibrio | parahaemolyticus | K5030 | 4807-4814 |
| Vibrio | parahaemolyticus | BB22OP | 4815-4822 |
| Vibrio | parahaemolyticus | VP250 | 4823-4830 |
| Vibrio | parahaemolyticus | IDH02189 | 4831-4838 |
| Vibrio | parahaemolyticus | CFSAN007460 | 4839-4845 |
| Vibrio | parahaemolyticus | CFSAN001611 | 4846-4852 |
| Vibrio | parahaemolyticus | CFSAN006129 | 4853-4859 |
| Vibrio | parahaemolyticus | CFSAN001619 | 4860-4865 |
| Vibrio | parahaemolyticus | 98-513-F52 | 4866-4872 |
| Vibrio | parahaemolyticus | CFSAN007451 | 4873-4880 |
| Vibrio | parahaemolyticus | CFSAN001614 | 4881-4887 |
| Vibrio | parahaemolyticus | 930 | 4888-4895 |
| Vibrio | parahaemolyticus | 12315 | 4896-4902 |
| Vibrio | parahaemolyticus | EN9701072 | 4903-4909 |
| Vibrio | parahaemolyticus | 876127 | 4910-4916 |
| Vibrio | parahaemolyticus | 872475 | 4917-4923 |
| Vibrio | parahaemolyticus | 857134 | 4924-4930 |
| Vibrio | parahaemolyticus | 480905 | 4931-4937 |
| Vibrio | parahaemolyticus | 926135 | 4938-4945 |
| Vibrio | parahaemolyticus | 860421 | 4946-4952 |
| Vibrio | parahaemolyticus | MAVP-45 | 4953-4959 |
| Vibrio | parahaemolyticus | S349-10 | 4960-4966 |
| Vibrio | parahaemolyticus | PMC58.7 | 4967-4974 |
| Vibrio | parahaemolyticus | PMC48 | 4975-4982 |
| Vibrio | parahaemolyticus | Gxw_7004 | 4983-4990 |
| Vibrio | parahaemolyticus | 450466 | 4991-4997 |
| Vibrio | parahaemolyticus | A0EZ608 | 4998-5004 |
| Vibrio | parahaemolyticus | A0EZ713 | 5005-5011 |
| Vibrio | parahaemolyticus | F30368 | 5012-5018 |
| Vibrio | parahaemolyticus | H18983 | 5019-5025 |
| Vibrio | parahaemolyticus | A3EZ711 | 5026-5032 |
| Vibrio | parahaemolyticus | A1EZ679 | 5033-5039 |
| Vibrio | parahaemolyticus | A3EZ799 | 5040-5046 |
| Vibrio | parahaemolyticus | C143 | 5047-5053 |
| Vibrio | parahaemolyticus | C148 | 5054-5060 |
| Vibrio | parahaemolyticus | A5Z853 | 5061-5068 |
| Vibrio | parahaemolyticus | A5Z905 | 5069-5075 |
| Vibrio | parahaemolyticus | CFSAN025062 | 5076-5084 |
| Vibrio | parahaemolyticus | CFSAN025066 | 5085-5093 |
| Vibrio | parahaemolyticus | CFSAN029654 | 5094-5102 |
| Vibrio | parahaemolyticus | CFSAN025060 | 5103-5111 |
| Vibrio | parahaemolyticus | CFSAN025052 | 5112-5120 |
| Vibrio | parahaemolyticus | CFSAN025057 | 5121-5129 |
| Vibrio | parahaemolyticus | CFSAN018754 | 5130-5137 |
| Vibrio | parahaemolyticus | GCSL_R12 | 5138-5144 |
| Vibrio | parahaemolyticus | GCSL_R30 | 5145-5151 |
| Vibrio | parahaemolyticus | GCSL_R32 | 5152-5158 |
| Vibrio | parahaemolyticus | GCSL_R57 | 5159-5167 |
| Vibrio | parahaemolyticus | GCSL_R77 | 5168-5174 |
| Vibrio | parahaemolyticus | GCSL_R138 | 5175-5181 |
| Vibrio | parahaemolyticus | GCSL_R149 | 5182-5188 |
| Vibrio | parahaemolyticus | CDC_K5324G | 5189-5195 |
| Vibrio | parahaemolyticus | CDC_K5437 | 5196-5202 |
| Vibrio | parahaemolyticus | CDC_K5280 | 5203-5209 |
| Vibrio | parahaemolyticus | CDC_K4558W | 5210-5216 |
| Vibrio | parahaemolyticus | CDC_K5009W | 5217-5224 |
| Vibrio | parahaemolyticus | CDC_K5067 | 5225-5231 |
| Vibrio | parahaemolyticus | CDC_K5346 | 5232-5238 |
| Vibrio | parahaemolyticus | CDC_K5345W | 5239-5245 |
| Vibrio | parahaemolyticus | CDC_K5579 | 5246-5252 |
| Vibrio | parahaemolyticus | CDC_K5629 | 5253-5259 |
| Vibrio | parahaemolyticus | CICESE-170 | 5260-5267 |
| Vibrio | parahaemolyticus | PMA12.14 | 5268-5275 |
| Vibrio | parahaemolyticus | VN-0293 | 5276-5282 |
| Vibrio | parahaemolyticus | xtf19 | 5283-5289 |
| Vibrio | parahaemolyticus | CFSAN001606 | 5290-5296 |
| Vibrio | parahaemolyticus | CFSAN001607 | 5297-5303 |
| Vibrio | parahaemolyticus | CFSAN001599 | 5304-5310 |
| Vibrio | parahaemolyticus | CFSAN018772 | 5311-5317 |
| Vibrio | parahaemolyticus | CFSAN018773 | 5318-5324 |
| Vibrio | parahaemolyticus | CFSAN018771 | 5325-5331 |
| Vibrio | parahaemolyticus | CFSAN022332 | 5332-5338 |
| Vibrio | parahaemolyticus | MAVP-L | 5339-5345 |
| Vibrio | parahaemolyticus | MAVP56 | 5346-5352 |
| Vibrio | parahaemolyticus | MAVP75 | 5353-5359 |
| Vibrio | anguillarum | S3 4/9 | 5360-5362 |
| Vibrio | parahaemolyticus | MAVP-46 | 5363-5369 |
| Vibrio | parahaemolyticus | MAVP-67 | 5370-5377 |
| Shewanella | sp. | 10N.286.48.A6 | 5378-5383 |
| Vibrio | lentus | 10N.261.55.E8 | 5384-5390 |
| Vibrio | diazotrophicus | 65.10M | 5391-5397 |
| Vibrio | crassostreae | 25_P_9 | 5398-5406 |
| Vibrio | parahaemolyticus | 16763 | 5407-5413 |
| Vibrio | parahaemolyticus | G1_8 | 5414-5421 |
| Vibrio | parahaemolyticus | S178 | 5422-5429 |
| Vibrio | parahaemolyticus | G1_4 | 5430-5437 |
| Vibrio | parahaemolyticus | F9_2 | 5438-5444 |
| Vibrio | parahaemolyticus | F3_9 | 5445-5452 |
| Vibrio | parahaemolyticus | F3_5 | 5453-5460 |
| Vibrio | parahaemolyticus | F3_2 | 5461-5468 |
| Vibrio | parahaemolyticus | F3_6 | 5469-5476 |
| Vibrio | parahaemolyticus | F2_8 | 5477-5484 |
| Vibrio | parahaemolyticus | VP900008 | 5485-5492 |
| Vibrio | parahaemolyticus | VP110008 | 5493-5500 |
| Vibrio | parahaemolyticus | VP840119 | 5501-5508 |
| Vibrio | parahaemolyticus | VP161407 | 5509-5516 |
| Vibrio | parahaemolyticus | VP170054 | 5517-5524 |
| Vibrio | parahaemolyticus | VP160968 | 5525-5532 |
| Vibrio | parahaemolyticus | VP830010 | 5533-5540 |
| Vibrio | parahaemolyticus | HZ13-102 | 5541-5549 |
| Photobacterium | sp. | CECT 9192 | 5550-5554 |

All of the bacteria described in Table A and Table B are accessible to those skilled in the art, as are their genomic sequences.

In embodiments, organisms that contain non-IF-3b systems are not expected to function with the described guide atypical guide RNAs, or at least it is considered that they would not exhibit enhanced transposition with the described atypical guide RNAs, which include atypical repeats and spacers from the organisms described in Table A.

In certain approaches of this disclosure expression vectors, such as plasmids, are used to produce one or more than one construct and/or component of the system, and any of their cloning steps or intermediates. A variety of suitable expression vectors known in the art can be adapted to produce components of this disclosure, including vectors that contain any desirable cargo, but in the context of other components described herein, and atypical repeats.

In embodiments, the compositions and methods of this disclosure are functional in a heterologous system. “Heterologous” as used herein means a system, e.g., a cell type, in which one or more of the components of the system are not produced without modification of the cells/system. A non-limiting embodiment of a heterologous system is any bacteria that is not Aeromonas salmonicida, including but not necessarily limited to Aeromonas salmonicida strain S44. In embodiments, a representative and non-limiting heterologous system is any type of E. coli.

In embodiments, any protein of this disclosure may be an Aeromonas salmonicida strain S44 protein, or a derivative thereof, with the exception that the TnsA protein is not produced by Aeromonas salmonicida strain S44, without modification, such as by recombinant engineering of the type described further herein. In embodiments, a described system is adapted from Aeromonas salmonicida S44 and exhibits greater transposition efficiency than a system adapted from Aeromonas hydrophila AFG_SD03.

In embodiments, the presently described systems that include gRNAs with atypical repeats and/or atypical spacers are used to direct blocks of genes to virtually any position in a bacterial genome, any episomal element, or a eukaryotic chromosome, in an orientation dependent fashion. In embodiments, the system is thus targeted to a sequence in a chromosome in a eukaryotic cell, or to a DNA extrachromosomal element in a eukaryotic cell, such as a DNA viral genome. Thus, the disclosure includes modifying eukaryotic chromosomes, and eukaryotic extrachromosomal elements. Accordingly, the type of extrachromosomal elements that can be modified according to the presently described compositions and methods are not particularly limited.

As known in the art, transposons are genetic elements that can move within a genome that appear to be found in all forms of life. In addition to the gRNAs discussed above, the present disclosure includes in part use of a version of the Tn7-like element where it has adapted the CRISPR-Cas system as a mechanism of targeting where the transposon moves, and further comprises mutations in certain Tn-related proteins that enhance CRISPR-Cas based editing using transposon proteins.

The present disclosure demonstrates that transposon and CRISPR-Cas systems can be used in cells to target insertion of the element into a single position adjacent to the match to the guide RNA in one orientation. This system has been recapitulated using recombinant approaches such that the transposon proteins and Cas proteins can be expressed in any position in the cell and they will act on the CRISPR array and transposon end-sequences found elsewhere in the cell.

Each set of genes described herein can also include a suitable xre gene that encodes a transcription regulator. Further, any of the tns genes, as further described herein, may comprise mutations such that tns genes encode proteins that are distinct from the proteins that are produced in nature, i.e., proteins that are produced by bacteria that have not been engineered to produce a modified Tns protein.

In particular, any cell of interest can be adapted to express the transposon and Cas proteins. For bacteria, this can be from an independently replicating plasmid or bacteriophage DNA or other element, or a vector that integrates into the genome, or an alternative delivery vector that is maintained or not maintained afterwards. In one embodiment, the user designs a guide RNA as described herein, such as a guide RNA that contains one, two, or more, atypical repeats, that contains a spacer that matches the sequences adjacent to the desired point of insertion. Designing guide RNAs according to this disclosure may take into account any sequence requirements that are dictated by any adjacent motifs (called PAM sequences). A sequence encoding the improved guide RNA is cloned into a delivery vector between repeats, at least one of which includes an atypical repeat (see, for example, FIGS. 3, 4, and 17).

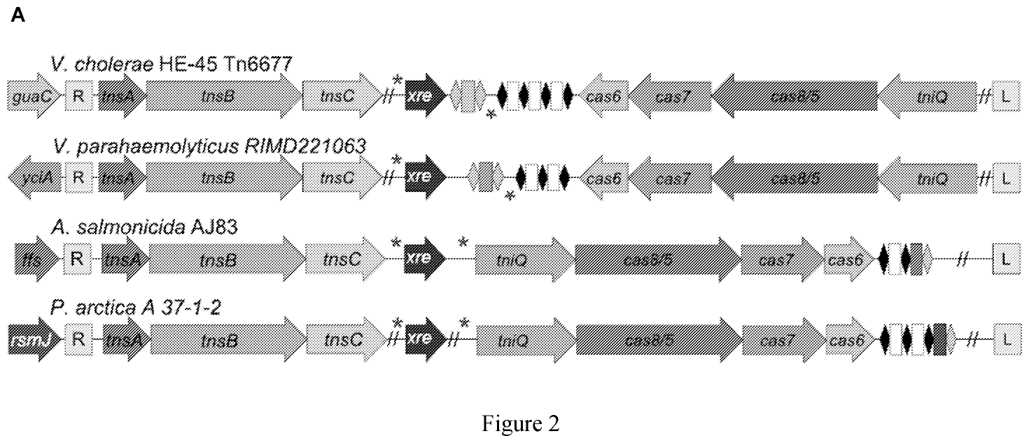

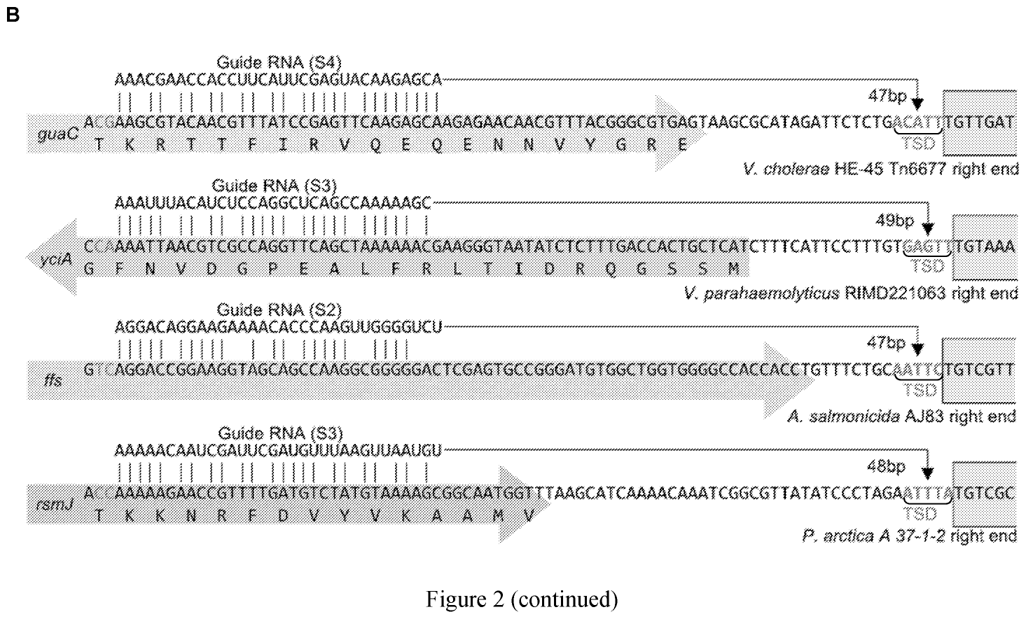

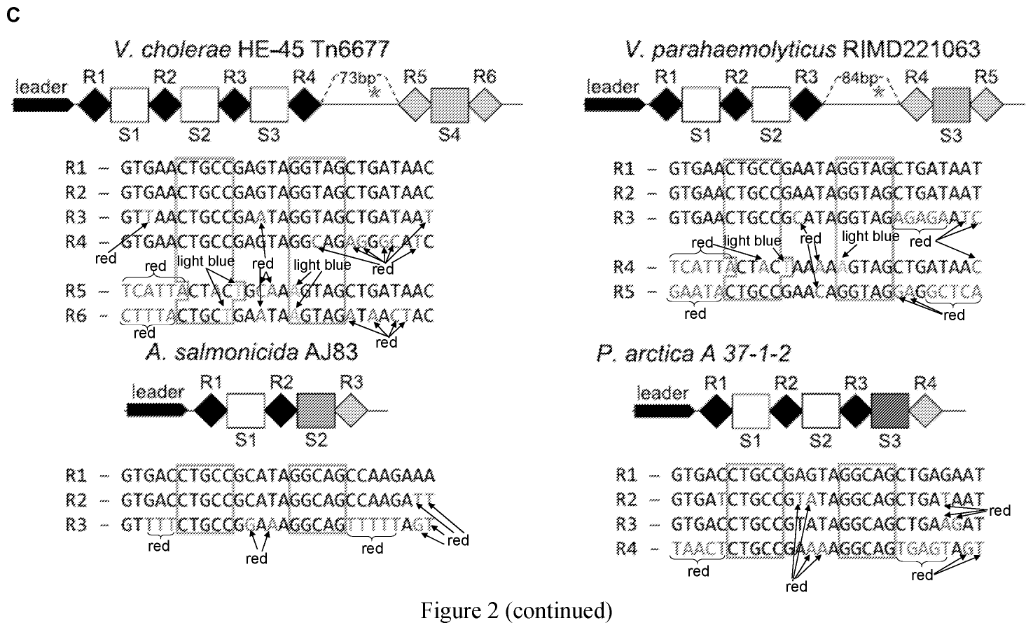

The disclosure includes using a least one tniQ gene, and accordingly two or more different tniQ genes may be used. tniQ genes produce a TniQ protein that is an optional part of the present system. Including this gene in the construct will direct transposition event into the one specific cognate site recognized by the TniQ protein. Without intending to be bound by any particular theory, it is considered that TniQ may also interact with the CRISPR/Cas and be required for guide RNA targeting. The genes of interest that are to be delivered into the bacterial strain or other suitable cell are cloned into a multicloning site (MCS) in the delivery vector using existing standard lab techniques (FIG. 2, panel B). The MCS is located between the left (L) and right (R) synthetic transposon end-sequences. If orientation of the final insertion is important for a particular embodiment, the right end of the element will be proximal to the match to the selected guide RNA. The delivery vector can be designed as a conditional vector that will not be maintained if desired. If desired, a selectable genetic marker can also be included in this vector. If the delivery vector will not be maintained, integration of the DNA by the targeted transposition process can be directly selected. If the efficiency is high enough, then this selectable marker is not needed.

This system can also be used to inactivate any gene in a prokaryotic or eukaryotic genome. Any one of many selectable markers can be included in the delivery vector to allow inactivation of a gene targeted by the guide RNA. This type of technology is broadly applicable to engineering new bacterial strains and eukaryotic cells for industry, research and therapeutic applications.

In contrast to existing CRISPR-based editing techniques, in addition to the presently described gRNAs, one advantage of the present systems is that no separate DNA break is used; instead the DNA fragment of interest is directly joined into the genome at one position determined by the user. Thus, in an embodiment, the disclosure provides for editing a target DNA without creating a double stranded DNA break.

In non-limiting demonstrations, the disclosure supports use of guide RNAs with atypical spacers in the systems described herein, and which include recombinantly produced proteins (the Cas proteins with or without TniQ are referred to in certain instances as ‘cascade”) can specifically recognize and bind to a DNA substrate that comprises a protospacer. As used in certain examples, cascade comprises Cas8-5 (encoding fused Cas proteins), Cas7, Cas6 and a guide RNA with or without one or more TniQ proteins. This combination illustrates cascade for variant I-F systems associated with Tn7-like elements.

It is expected that the results that embodiments of the disclosure described as follows can be produced with any guide RNA that is an aspect of this disclosure, e.g., the typical 5′ end or 3′ end that forms the guide RNA can be engineered to form a sequence that is the RNA equivalent of an atypical repeat, and exhibits of enhanced activity in the resulting guide RNA. It is considered that including sequences matching atypical repeats in such guide RNAs would improve the results in the following description.

For example, as shown in the examples and figures of this disclosure, in vitro binding of cascade occurs with specificity to a DNA substrate comprising a protospacer, to which the cascade complex is directed using a suitable guide RNA, which may be adapted to use the presently provided guide RNAs with atypical repeats. Likewise, the figures and examples demonstrate copurification of a complex comprising TniQ and cascade. Thus, the disclosure shows that recombinantly produced TinQ and cascade form a physical association. Moreover, as described in the foregoing description and figures, the disclosure demonstrates functionality of the system in a living heterologous system (illustrated using E. coli). In particular, the figures, show guided transposition that is specific for a particular location in a conjugal plasmid, and that this transposition is PAM specific. In particular, in endogenous Aeromonas, the insertion was 48 base pairs from the protospacer. Thus, the disclosure demonstrates functionality of the system using recombinant approaches in living cells that do not, without modification as described herein, produce a directed transposition event. Additionally, the disclosure demonstrates transposition from one location in a chromosome to another location in the chromosome, results which are also obtained in a heterologous system, using E. coli as a representative example.

In embodiments, systems of this disclosure include a DNA cargo for insertion into a eukaryotic chromosome or extrachromosomal element, or in the case of prokaryotes, a chromosome or a plasmid. Thus, instead of transposing an existing segment of a genome in the manner in which transposons ordinarily function, the disclosure provides for insertion of DNA cargo that can be selected by the user of the system. The DNA cargo may be provided, for example, as a circular or linear DNA molecule. The DNA cargo can be introduced into the cell prior to, concurrently, or after introducing a system of the disclosure into a cell. The sequence of the DNA cargo is not particularly limited, other than a requirement for suitable right and left ends that are recognized by proteins of the system. The right and left end sequences that are required for recognition are typically from about 90-150-bp in length. As is known in the art, such 90-150 bp length comprises multiple 22 bp binding sites for the TnsB transposase in the element in each of the ends that can be overlapping or spaced.

In embodiments, the transposable DNA cargo sequence is transposed into the chromosome or extrachromosomal element within a 5 nucleotide sequence that includes the nucleotide that is located 47 nucleotides 3′ relative to the 3′ end of the protospacer. In embodiments, a DNA cargo insertion comprises an insertion at the center of a 5 bp target site duplication (TSD). Thus, by providing a guide RNA as described herein that is cognate to the protospacer, precise and PAM specific integration of a DNA cargo can be achieved. In embodiments, the PAM comprises or consists of TACC or CC or variants of NC and CN, including any of CG, CA and TC, as illustrated in non-limiting embodiments in FIG. 2b.

The transposon and Cas genes can be expressed from any of a wide variety of existing mechanism that can replicate separately in the cell or be integrated into the host cell genome. Alternatively, they could be expressed transiently from an expression system that will not be maintained. In embodiments, the proteins themselves could be directly transformed into the host strain to allow their function. The disclosure allows for multiple copies of distinct transposon gene cassettes, multiple copies of Cas genes, CRISPR arrays, and multiple distinct cargo coding sequences to be introduced and to modify genetic material in the same cell. In embodiments a first set of transposon genes tnsA, tnsB, tnsC, and optionally one or more tniQ genes, Cas genes cas8f, cas5f cas7f, and cas6f and an xre gene, and a sequence encoding at least a first guide RNA, as described herein, that is functional with proteins encoded by the Cas genes, wherein at least one of the first set of transposon genes, the Cas genes, or the sequence encoding the first guide RNA are present within and/or are encoded by a recombinant polynucleotide that is introduced into bacteria, or eukaryotic cells. The disclosure thus includes second, third, fourth, fifth, or more copies of distinct transposon genes, Cas genes, and distinct cargo coding sequences

In one example, the disclosure provides a system for modifying a genetic target in bacteria and/or eukaryotic cells. The system comprises a first set of transposon genes tnsA, tnsB, tnsC, and optionally one or more tniQ, Cas genes cas8f cas5f cas7f and cas6f, and an xre gene encoding a transcription regulator, and a sequence encoding a first guide RNA, as described herein, that is functional with proteins encoded by the Cas genes, wherein at least one of the first set of transposon genes, the Cas genes, and/or or the sequence encoding the first guide RNA are present within and/or are encoded by a recombinant polynucleotide. Without intending to be constrained by any particular theory, it is considered that the xre gene, while annotated as a transcriptional regulator, can also make transposition complexes described herein more efficient.

In embodiments, one or more of the tns genes, and therefore the proteins they encode, are modified, as described in more detail below. From this disclosure, and other information known to those skilled in the art, homologous proteins can be recognized, aligned, and amino acid changes in the proteins can be made such that the proteins function in a manner similar to those described herein. All such homologous proteins and mutations thereof are included in this disclosure. The disclosure also includes combinations of naturally occurring genes and proteins, with the exception that one or more of the naturally occurring sequences may be expressed from one or more recombinant vectors. In embodiments, homologous proteins are from any bacteria, including but not limited to Proteobacteria.

Certain embodiments of mutations in proteins that are included in the disclosure are provided below. The mutations can be in any one or any combination of proteins encoded by the tnsA gene, the tnsB gene, and the tnsC gene.

In embodiments, the Tns proteins that are provided by this disclosure comprise mutations relative to a wild type sequence. A “wild type” sequence as used herein means a sequence that preexists in nature without experimentally engineering a change in the sequence. In embodiments, a wild type sequence is the sequence of a transposition element, a non-limiting example of which is the sequence of Aeromonas salmonicida strain S44 plasmid pS44-1, which can be accessed via accession no. CP022176 (Version CP022176.1), such as via www.ncbi.nlm.nih.gov/nuccore/CP022176.

In embodiments, the mutations described in i), ii) and iii) below provide for an increase in transposition frequency that is similar or greater than a value obtained from a control construct. In embodiments, the control construct comprises one or more tns genes in which a mutation described herein is not present, and/or the control comprises a guide RNA with one or more segments that recognize a typical repeat, wherein the increased transposition efficiency is achieved with a guide RNA of this disclosure which includes one or more sequences that recognize atypical repeats. In embodiments, a control transposition frequency is a frequency exhibited by a transposition element from Aeromonas hydrophila strain AFG_SD03, which can be identified from Accession PUTQ01000019 (Version PUTQ01000019.1), and which comprises representative amino acid sequences described below, except for the indicated mutations. The pertinent sequence of Aeromonas hydrophila strain AFG_SD03 can be accessed via, for example, www.ncbi.nlm.nih.gov/nuccore/1427716682. The Aeromonas salmonicida Cas8/5 amino acid sequence is available under accession number ASI25653, www.ncbi.nlm.nih.gov/protein/ASI25653.1; Aeromonas salmonicida Cas7 amino acid sequence is available under accession number ASI25654, www.ncbi.nlm.nih.gov/protein/ASI25654.1; Aeromonas salmonicida Cas6 amino acid sequence is available under accession number ASI25655, www.ncbi.nlm.nih.gov/protein/ASI25655.1. In an embodiment, the control comprises a system that that is present on the Tn6677 element, as further described below.

In embodiments, assuming only for illustration, a frequency of transposition of 0.0001% is a control value because transposition efficiency was not able to be measured in the representative assays, (e.g., hypothetically only one in 100,000 cells into which a presently described system using a wild type TnsA protein experience a transposition event). In this regard, the present disclosure provides for a 1 fold to 200 fold increase in transposition efficiency, inclusive, and including all numbers and ranges of to the first decimal point there between, relative to a control frequency of transposition. In embodiments, transposition efficiency can be equated to insertion of a user supplied DNA template that is inserted into a selected location in a DNA substrate.

In embodiments, the CRISPR guide RNAs and systems provided herein effect a modification in a DNA target sequence, for example, insertion of a sequence into the DNA target sequence via transposition. The DNA target sequence may comprise a DNA cargo sequence for insertion. In some embodiments, the guide RNAs facilitates increased efficiency of the modification as compared to efficiency of modification using a control guide RNA. In some embodiments, the guide RNA is an atypical guide RNA and the modification is effected with a type I-F3b CRISPR complex as described herein, and a control guide RNA is a guide RNA not comprising a diverged repeat as herein described (e.g., a “typical” guide RNA). In some embodiments, the modification (e.g., transposition) efficiency is at least 1.5 fold greater than a control modification efficiency using a control guide RNA. In some embodiments, the modification efficiency is at least 2 fold greater than a control modification efficiency using a control guide RNA. In some embodiments, the modification efficiency is at least 4 fold greater than a control modification efficiency using a control guide RNA. In embodiments, the disclosure facilitates an increase of transposition efficiency relative to a control, such as transposition from a chromosome to a plasmid, of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, fold greater than a control value. Similar transposition efficiency can be determined for transposition events where the transposition comprises transposing an element in cis, e.g., transposition from one location in a chromosome to a different location in the same chromosome.

i) In one embodiment of this disclosure, the tnsA gene comprises a change in sequence such that at least one amino acid in the TnsA protein encoded by the tnsA gene is changed relative to its wild type sequence. In an embodiment, the change in the TnsA protein comprises a change of Ala at position 125 of an Aeromonas salmonicida TnsA protein, wherein optionally the change is to an Asp, or is a homologous change in a homologous TnsA protein. A representative TnsA amino acid sequence is provided below. In this regard, we have demonstrated that this construct can introduce numerous insertions, but without the change insertions approximate background levels, or are undetectable.

ii) In embodiments, the disclosure includes a tnsB gene comprising a change in sequence such that at least one amino acid in the TnsB protein encoded by the tnsB gene is changed relative to its wild type sequence. In an embodiment, the change in the TnsB protein comprises a change of amino acid position 167 of an Aeromonas salmonicida TsnB protein, wherein optionally the change is a Ser, or is a homologous change in a homologous position of a homologous TnsB protein. Representative TnsB amino acid sequences are provided below.

iii) As with the TnsA and TnsB proteins, in embodiments, the disclosure includes a modified tnsC gene that comprises a change in sequence such that at least one amino acid in the TnsC protein encoded by the tnsC gene is changed relative to its wild type sequence. In embodiments, the change is optionally located in a TnsC Walker B motif. In embodiments, the change in a Walker B motif is, for example, in position 135, 136, 137, 138, 139, or 140 of the Aeromonas salmonicida TnsC protein, a representative example of which is shown below. In one embodiment, the change is to an amino acid at position 140 in the TnsC protein, wherein, for example, amino acid 140 is change to an Ala or Gln, or a homologous change in a homologous position of a homologous TnsC protein is made.

iii) the tnsC gene comprises a change in sequence such that at least one amino acid in the TnsC protein encoded by the tnsC gene is changed relative to its wild type sequence, wherein the change is optionally in a TnsC Walker B motif.

In embodiments, any composition, system, or method of this disclosure may be performed in the absence of any TnsE transposon protein. TnsE transposon proteins are known in the art. In a non-limiting embodiment, any composition, system, and/or method of this disclosure may be performed in the absence of, and/or without participation of, an E. coli TnsE protein that comprises or consists of the following amino acid sequence:

(SEQ ID NO: 5676)
MVRLATFNDNVQVVHIGHLFRNSGHKEWRIFVWFNPMQERKWTRFTHLP
LLSRAKVVNSTTKQINKADRVIEFEASDLQRAKIIDFPNLSSFASVRNK
DGAQSSFIYEAETPYSKTRYHIPQLELARSLFLINSYFCRSCLSSTALQ
QEFDVQYEVERDHLEIRILPSSSFPKGALEQSAVVQLLVWLFSDQDVMD
SYESIFRHYQQNREIKNGVESWCFSFDPPPMQGWKLHVKGRSSNEDKDY
LVEEIVGLEINAMLPSTTAISHASFQEKEAGDGSTQHIAVSTESVVDDE
HLQLDDEETANIDTDTRVIEAEPTWISFSRPSRIEKSRRARKSSQTILE
KEEATTSENSNLVSTDEPHLGGVLAAADVGGKQDATNYNSIFANRFAAF
DELLSILKTKFACRVLFEETLVLPKVGRSRLHLCKDGSPRVIKAVGVQR
NGSEFVLLEVDASDGVKMLSTKVLSGVDSETWRNDFEKIRRGVVKSSLN
WPNSLFDQLYGQDGHRGVNHPKGLGELQVSREDMEGWAERVVREQFTH.

In embodiments, any composition, system, and/or method of this disclosure may be performed in the absence of, and/or without participation of any TnsE protein that is a homologue of the foregoing sequence, but is from a type of bacteria that is not E. coli.

Non-limiting embodiments of amino acid sequences comprising mutations and/or locations of mutations are described herein, and by way of the following amino acid sequences and accession numbers. Enlarged, bold and italicized amino acids signify non-limiting examples of mutations that are encompassed by this disclosure. Enlarged sequences are locations where other mutations may be made, and are also included in this disclosure.

TnsA (A125D) Change from Aeromonas salmonicida Strain S44 Plasmid pS44-1 or TnsA Exact from Aeromonas Hydro Hila Strain AFG_SD03

(SEQ ID NO: 5677)
MYRRHLKHSRVKNLFKFVSAKMNTVFTVESALEFDTCFHLEYSPSVKFY
EAQPEGFYYEFAGRQCPYTPDFRLVDQNDSVSFLEIKPSDKVADPDFLH
RFPLKQQRAIELSSPLKLVTEKQIRI [symbol] PILGNLKLLHRYSGFQSFTPLH
MQLLGLVQKLGRVSLLRLSDSIDAPPEEVLASALSLIARGIMQSDLTVQ
KIGISSFVWAGGHSGIDHG

TnsB (from Aeromonas salmonicida Strain S44 Plasmid S44-1)

(SEQ ID NO: 5678)
MDKHNGGLFEDEFVIPQPSTSTSPIDAIQAVLPATVDSFPYVLKVEALH
RRDYILWVEKNLAGGWTEKNLTPLLADAALVLPPPTPNWRTLARWRKIY
IQHGRKLVSLIPKHQAKGNARSRLPPSDELFFEQAVHRYLVGEQPSIAS
AFQLYSDSIRIENLGVVEN [symbol] IKTISYMAFYNRIKKLPAYQVMKSRKGSY
IADVEFKAIASHKPPSRIMERVEIDHTPLDLLLLDDDLLVPLGRPSLTL
LIDAYSHCVVGFNLNFNQPSYESVRNALLSSISKKDYVKNKYPSIEHEW
PCYGKPETLVVDNGVEFWSASLAQSCLELGINIQYNPVRKPWLKPMIER
MFGIINRKLLEPIPGKTFSNIQEKGDYDPQKDAVMRFSTFLEIFHHWVI
DVYHYEPDSRYRYIPIISWQHGNKDAPPAPIIGDDLTKLEVILSLSLHC
THRRGGIQRYHLRYDSDELASYRMNYPDQTRGKRKVLVKLNPRDISYVY
VFLEDLGSYIRVPCIDPIGYTKGLSLQEHQINVKLHRDFINEQMDVVSL
SKARIYLNDRIKNELIEVRRNIRQRNVKGVNKIAKYRNVGSHAETSIVH
ELNHPATNEVISKMESASQPEHCDDWDNFTSGLEPY

TnsB (P167S) Change from Aeromonas salmonicida Strain S44 Plasmid pS44-1

(SEQ ID NO: 5679)
MDKHNGGLFEDEFVIPQPSTSTSPIDAIQAVLPATVDSFPYVLKVEALH
RRDYILWVEKNLAGGWTEKNLTPLLADAALVLPPPTPNWRTLARWRKIY
IQHGRKLVSLIPKHQAKGNARSRLPPSDELFFEQAVHRYLVGEQPSIAS
AFQLYSDSIRIENLGVVENSIKTISYMAFYNRIKKLPAYQVMKSRKGSY
IADVEFKAIASHKPPSRIMERVEIDHTPLDLLLLDDDLLVPLGRPSLTL
LIDAYSHCVVGFNLNFNQPSYESVRNALLSSISKKDYVKNKYPSIEHEW
PCYGKPETLVVDNGVEFWSASLAQSCLELGINIQYNPVRKPWLKPMIER
MFGIINRKLLEPIPGKTFSNIQEKGDYDPQKDAVMRFSTFLEIFHHWVI
DVYHYEPDSRYRYIPIISWQHGNKDAPPAPIIGDDLTKLEVILSLSLHC
THRRGGIQRYHLRYDSDELASYRMNYPDQTRGKRKVLVKLNPRDISYVY
VFLEDLGSYIRVPCIDPIGYTKGLSLQEHQINVKLHRDFINEQMDVVSL
SKARIYLNDRIKNELIEVRRNIRQRNVKGVNKIAKYRNVGSHAETSIVH
ELNHPATNEVISKMESASQPEHCDDWDNFTSGLEPY

TnsC (from Aeromonas salmonicida Strain S44 Plasmid pS44-1)

(SEQ ID NO: 5680)
MDLSCHDADKLRSFIECYVETPLLRAIQEDFDRLRFNKQFAGEPQCMLL
TGDTGTGKSSLIRHYAAKHPEQVRHGFIHKPLLVSRIPSRPTLESTMVE
LLKDLGQFGSSDRIHKSSAESLTEALIKCLKRCETE [symbol] FQELIEN
KTREKRNQIANRLKYISETAKIPIVLVGMPWATKIAEEPQWSSRLLIRR
SIPYFKLSDDRENFIRLIMGLANRMPFETQARLETKHTIYALFAACYGS
LRALKQLLDESVKQALAAHAETLKHEHIAVAYALFYPDQVNPFLQPIDE
IKACEVKQYSRYEIDAAGKEEVLNPLQFTDKIPISQLLKKR

TnsC (E140A) Change from Aeromonas salmonicida Strain S44 Plasmid pS44-1

(SEQ ID NO: 5681)
MDLSCHDADKLRSFIECYVETPLLRAIQEDFDRLRFNKQFAGEPQCMLL
TGDTGTGKSSLIRHYAAKHPEQVRHGFIHKPLLVSRIPSRPTLESTMVE
LLKDLGQFGSSDRIHKSSAESLTEALIKCLKRCETE [symbol] FQELIEN
KTREKRNQIANRLKYISETAKIPIVLVGMPWATKIAEEPQWSSRLLIRR
SIPYFKLSDDRENFIRLIMGLANRMPFETQARLETKHTIYALFAACYGS
LRALKQLLDESVKQALAAHAETLKHEHIAVAYALFYPDQVNPFLQPIDE
IKACEVKQYSRYEIDAAGKEEVLNPLQFTDKIPISQLLKKR

TnsC (E140Q) Change from Aeromonas salmonicida Strain S44 Plasmid pS44-1

(SEQ ID NO: 5682)
MDLSCHDADKLRSFIECYVETPLLRAIQEDFDRLRFNKQFAGEPQCMLL
TGDTGTGKSSLIRHYAAKHPEQVRHGFIHKPLLVSRIPSRPTLESTMVE
LLKDLGQFGSSDRIHKSSAESLTEALIKCLKRCETE [symbol] FQELIEN
KTREKRNQIANRLKYISETAKIPIVLVGMPWATKIAEEPQWSSRLLIRR
SIPYFKLSDDRENFIRLIMGLANRMPFETQARLETKHTIYALFAACYGS
LRALKQLLDESVKQALAAHAETLKHEHIAVAYALFYPDQVNPFLQPIDE
IKACEVKQYSRYEIDAAGKEEVLNPLQFTDKIPISQLLKKR

Xre (gene 91099.91428, Locus tag CE463_00475) from Aeromonas salmonicida strain S44 plasmid pS44-1. The disclosure includes homologous Xre sequences. The sequence below is identical to the Xre protein in Aeromonas hydrophila strain AFG_SD03.

(SEQ ID NO: 5683)
MTNPLPIRLKAARKATGLTQQQLGIRLGMEQSTASARMNQYEKGKHAPD
YQTMQRIAQELGYPVAYFYCDDELLAELICMMAKLSEEKQRELLQQLSV
TEYAESRDSAE

In addition to any of the foregoing mutations, the disclosure also includes additional amino acid changes, such as changes in TnsC, which may include gain-of-activity mutations, in canonical Tn7 (e.g., homologous proteins), including but not necessarily limited to TnsABC(A225V), TnsABC(E233K), TnsABC(E233A), and TnsABC(E233Q).

In one aspect the disclosure includes a kit comprising one or more expression vector(s) that encodes one or more Cas or other enzymes described herein. The expression vector in certain approaches includes a cloning site, such as a poly-cloning site, such that any desirable cargo gene(s) can be cloned into the cloning site to be expressed in any target cell into which the system is introduced or already comprises. The kit can further comprise one or more containers, printed material providing instructions as to how to use make and/or use the expression vector to produce suitable vectors, and reagents for introducing the expression vector into cells. The kits may further comprise one or more bacterial strains for use in producing the components of the system. The bacterial strains may be provided in a composition wherein growth of the bacteria is restricted, such as a frozen culture with one or more cryoprotectants, such as glycerol. In embodiments, the kit comprises a vector for expression of a guide RNA comprising a user selected spacer. The expression vector encodes at least a portion of a guide RNA that contains at least one atypical repeat. The expression vector can be configured such that a user selected spacer can be cloned into the expression vector adjacent to at least one atypical repeat. A cloning site can be configured such that a pair of atypical repeats will flank the spacer that is cloned into the expression vector.

In another aspect the disclosure comprises delivering to cells a DNA cargo via a system of this disclosure. The method generally comprises introducing one or more polynucleotides of this disclosure, or a mixture or proteins and polynucleotides encoding the proteins, which may be also provided with RNA polynucleotides, such as the presently described guide RNAs, into one or more bacterial or eukaryotic cells, whereby the Cas and transposon enzymes/proteins are expressed and editing of the chromosome or another DNA target by a combination of the Cas enzymes and the transposon occurs.

In non-limiting embodiments, this disclosure is considered to be suitable for targeting eukaryotic cells, and any microorganism that is susceptible to editing by a system as described herein. In embodiments the microorganism comprises bacteria that are resistant to one or more antibiotics, whereby the editing by the present system kills or reduces the growth of the antibiotic-resistant bacteria, and/or the system sensitizes the bacteria to an antibiotic by, for example, use of cargo that targets an antibiotic resistance gene, which may be present on a chromosome or a plasmid. The disclosure is thus suitable for targeting bacterial chromosomes or episomal elements, e.g., plasmids. In embodiments, a modification of a bacterial chromosome or plasmid causes the bacteria to change from pathogenic to non-pathogenic.

In embodiments, bacteria are killed. In embodiments, one or all of the components of a system described herein can be provided in a pharmaceutical formulation. Thus, in embodiments, DNA, RNA, proteins, and combinations thereof can be provided in a composition that comprises at least one pharmaceutically acceptable additive.

In embodiments, the method of this disclosure is used to reduce or eradicate bacterial cells, and may be used to reduce or eradicate persister bacteria and/or dormant viable but non-culturable (VBNC) bacteria from an individual or an inanimate surface, or a food substance.

In embodiments, and as noted above, the disclosure is considered suitable for editing eukaryotic cells. In embodiments, eukaryotic cells that are modified by the approaches of this disclosure are totipotent, pluripotent, multipotent, or oligopotent stem cells when the modification is made. In embodiments, the cells are neural stem cells. In embodiments, the cells are hematopoietic stem cells. In embodiments, the cells are leukocytes. In embodiments, the leukocytes are of a myeloid or lymphoid lineage. In embodiments, the cells are embryonic stem cells, or adult stem cells. In embodiments, the cells are epidermal stem cells or epithelial stem cells. In embodiments, the cells are cancer cells, or cancer stem cells. In embodiments, the cells are differentiated cells when the modification is made. In embodiments, the cells are mammalian cells. In embodiments, the cells are human, or are non-human animal cells. In embodiments, the non-human eukaryotic cells comprise fungal, plant or insect cells. In one approach the cells are engineered to express a detectable or selectable marker, or a combination thereof.

In embodiments, the disclosure includes obtaining cells from an individual, modifying the cells ex vivo using a CRISPR system as described herein, and reintroducing the cells or their progeny into the individual for prophylaxis and/or therapy of a condition, disease or disorder, or to treat an injury, trauma or anatomical defect. In embodiments, the cells modified ex vivo as described herein are used autologously.

In embodiments, cells modified according to this disclosure are provided as cell lines. In embodiments, the cells are engineered to produce a protein or other compound, and the cells themselves or the protein or compound they produce is used for prophylactic or therapeutic applications.

In various embodiments, the modification introduced into eukaryotic cells according to this disclosure is homozygous or heterozygous. In embodiments, the modification comprises a homozygous dominant or homozygous recessive or heterozygous dominant or heterozygous recessive mutation correlated with a phenotype or condition, and is thus useful for modeling such phenotype or condition. In embodiments a modification causes a malignant cell to revert to a non-malignant phenotype.

In certain aspects the disclosure includes a pharmaceutical formulation comprising one or more components of a system described herein. A pharmaceutical formulation comprises one or more pharmaceutically acceptable additives, many of which are known in the art. In some embodiments, the pharmaceutical compositions comprise a pharmaceutically acceptable carrier suitable for administration to humans. In some embodiments, the pharmaceutical compositions comprise a pharmaceutically acceptable carrier suitable for intraocular injection. In some embodiments, the pharmaceutical compositions comprise a pharmaceutically acceptable carrier suitable for topical application. In some embodiments, the pharmaceutical compositions comprise a pharmaceutically acceptable carrier suitable for intravenous injection. In some embodiments, the pharmaceutical compositions comprise and a pharmaceutically acceptable carrier suitable for injection into arteries. In some embodiments, the pharmaceutical composition is suitable for oral or topical administration. All of the described routes of administration are encompassed by the disclosure.

In embodiments, expression vectors, proteins, RNPs, polynucleotides, and combinations thereof, can be provided as pharmaceutical formulations. A pharmaceutical formulation can be prepared by mixing the described components with any suitable pharmaceutical additive, buffer, and the like. Examples of pharmaceutically acceptable carriers, excipients and stabilizers can be found, for example, in Remington: The Science and Practice ofPharmacy (2005) 21st Edition, Philadelphia, Pa. Lippincott Williams & Wilkins, the disclosure of which is incorporated herein by reference. Further, any of a variety of therapeutic delivery agents can be used, and include but are not limited to nanoparticles, lipid nanoparticle (LNP), exosomes, and the like. In embodiments, a biodegradable material can be used. In embodiments, poly(lactide-co-galactide) (PLGA) is a representative biodegradable material. In embodiments, any biodegradable material, including but not necessarily limited to biodegrable polymers. As an alternative to PLGA, the biodegradable material can comprise poly(glycolide) (PGA), poly(L-lactide) (PLA), or poly(beta-amino esters). In embodiments, the biodegradable material may be a hydrogel, an alginate, or a collagen. In an embodiment the biodegradable material can comprise a polyester a polyamide, or polyethylene glycol (PEG). In embodiments, lipid-stabilized micro and nanoparticles can be used.

In certain approaches, compositions of this disclosure, including the described systems, and cells modified using the described systems, are used for treatment of condition or disorder in an individual in need thereof. The term “treatment” as used herein refers to alleviation of one or more symptoms or features associated with the presence of the particular condition or suspected condition being treated. Treatment does not necessarily mean complete cure or remission, nor does it preclude recurrence or relapses. Treatment can be effected over a short term, over a medium term, or can be a long-term treatment, such as, within the context of a maintenance therapy. Treatment can be continuous or intermittent.

In embodiments, a system of this disclosure is administered to an individual in a therapeutically effective amount. In embodiments, a therapeutically effective amount of a composition of this disclosure is used. The term “therapeutically effective amount” as used herein refers to an amount of an agent sufficient to achieve, in a single or multiple doses, the intended purpose of treatment. The amount desired or required will vary depending on the particular compound or composition used, its mode of administration, patient specifics and the like. Appropriate effective amounts can be determined by one of ordinary skill in the art informed by the instant disclosure using routine experimentation. For example, a therapeutically effective amount, e.g., a dose, can be estimated initially either in cell culture assays or in animal models. An animal model can also be used to determine a suitable concentration range, and route of administration. Such information can then be used to determine useful doses and routes for administration in humans, or to non-human animals. A precise dosage can be selected by in view of the patient to be treated. Dosage and administration can be adjusted to provide sufficient levels of components to achieve a desired effect, such as a modification in a threshold number of cells. Additional factors which may be taken into account include the particular gene or other genetic element involved, the type of condition, the age, weight and gender of the patient, desired duration of treatment, method of administration, time and frequency of administration, drug combination(s), reaction sensitivities, and tolerance/response to therapy. In certain embodiments, a therapeutically effective amount is an amount that reduces one or more signs or symptoms of a disease, and/or reduces the severity of the disease. A therapeutically effective amount may also inhibit or prevent the onset of a disease, or a disease relapse. In embodiments, cells modified according to this disclosure are administered to an individual in need thereof in a therapeutically effective amount.

In embodiments, the disclosure comprises providing a treatment to an individual in need thereof by introducing a therapeutically effective amount a composition of this disclosure, or modified cells as described herein to the individual, wherein the cells comprising the DNA insertion treats, alleviates, inhibits, or prevents the formation of one or more conditions, diseases, or disorders. In embodiments, the cells are first obtained from the individual, modified according to this disclosure, and transplanted back into the individual. In embodiments, allogenic cells can be used. In embodiments, the modified eukaryotic cells can be provided in a pharmaceutical formulation, and such formulations are included in the disclosure.

In embodiments, a described system of this disclosure is introduced into one or more prokaryotic or eukaryotic cells. In embodiments, the prokaryotic cells comprise or consist of gram positive, or gram negative bacteria. The bacteria may be non-pathogenic, or pathogenic. In embodiments, a described system is introduced into prokaryotic cells (e.g., bacterial or archaeal cells) in the context of a host, e.g., a human, animal, or plant host, e.g., the bacteria are a component of a host's microbiome or are an abnormal component of a microbiome, e.g., a pathogen. In some embodiments, delivery of a system described herein results in the stable formation of a recombinant microorganism. In some embodiments, a recombinant microorganism as generated by a system described herein results in the production of an enzyme or metabolite that can alter the health or metabolism of a host, e.g., a human host. In some embodiments, delivery of a system described herein results in the inactivation of virulence determinants of a microorganism, e.g., antibiotic resistance or toxin production. In some embodiments, delivery of a system described herein results in killing of the recipient cell. The system may kill some or all of the cells, or render the cells non-pathogenic and/or sensitive to one or more antibiotics. In embodiments, the bacteria are used as a component of a food or beverage product, including but not limited to fermented food and beverages, and dairy products. In embodiments, such bacteria comprise Lactic acid bacteria. In embodiments, selective delivery to a specific type of bacteria is used by way of a bacteriophage or packaged phagemids that can express all or some of the described components, but wherein the bacteriophage exhibits a specific tropism for a particular type of bacteria. In some embodiments, a delivery vehicle provides only partial specificity towards targeting particular cells, and additional specificity is provided by the choice of DNA sequence being targeted.

In embodiments, the described systems are introduced into eukaryotic cells. Such cells include but are not necessarily limited to animal cells, fungi such as yeasts, protists, algae, and plant cells.

In embodiments, the disclosure provides one or more cells, wherein DNA in the cells comprises at least one inserted DNA insertion template. The described cells may be any prokaryotic or eukaryotic cells. Accordingly, the disclosure also provides one or more cells that comprise an inserted DNA sequence.

In embodiments, the eukaryotic cells comprise animal cells, which may comprise mammalian or avian cells, or insect cells. In embodiments, the mammalian cells are human or non-human mammalian cells. In embodiments, compositions of this disclosure are administered to avian animals, or to a canine, a feline, an equine animal, or to cattle, including but not limited to dairy cattle.

In embodiments, the cells that are modified by the approaches of this disclosure are totipotent, pluripotent, multipotent, or oligopotent stem cells when the modification is made. In embodiments, the cells are neural stem cells. In embodiments, the cells are hematopoietic stem cells. In embodiments, the cells are leukocytes. In embodiments, the leukocytes are of a myeloid or lymphoid lineage. In embodiments, the cells are embryonic stem cells, or adult stem cells. In embodiments, the cells are epidermal stem cells or epithelial stem cells. In embodiments, the cells are cancer cells, or cancer stem cells. In embodiments, the cells are differentiated cells when the modification is made.

In embodiments, the disclosure includes obtaining cells from an individual, modifying the cells ex vivo using a system as described herein, and reintroducing the cells or their progeny into the individual or a immunologically matched individual for prophylaxis and/or therapy of a condition, disease or disorder, or to treat an injury, trauma or anatomical defect. In embodiments, the cells modified ex vivo as described herein are autologous cells. In embodiments, the cells are provided as cell lines. In embodiments, the cells are engineered to produce a protein or other compound, and the cells themselves and/or the protein or compound they produce is used for prophylactic or therapeutic applications.

In embodiments, eukaryotic cells made according to this disclosure can be used to create transgenic, non-human organisms.

In embodiments, one or more modified cells according to this disclosure may be used to perform a gene-drive in a population of animals, including but not necessarily limited to insects.

In embodiments, the one or more cells into which a described system is introduced comprises a plant cell. The term “plant cell” as used herein refers to protoplasts, gamete producing cells, and includes cells which regenerate into whole plants. Plant cells include but are not necessarily limited to cells obtained from or found in: seeds, suspension cultures, embryos, meristematic regions, callus tissue, leaves, roots, shoots, gametophytes, sporophytes, pollen, and microspores. Plant cells can also be understood to include modified cells, such as protoplasts, obtained from the aforementioned tissues. Plant products made according to the disclosure are included.

In embodiments, the disclosure provides an article of manufacture, which may comprise a kit. In embodiments, the article of manufacture may comprise one or more cloning vectors. The one or more cloning vectors may encode any one or combination of proteins and polynucleotides described herein. The cloning vectors may be adapted to include, for example, a multiple cloning site (MCS), into which a sequence encoding any protein or polynucleotide, such as any desired targeting RNA, may be introduced. An article of manufacture may include one or more sealed containers that contain any of the aforementioned components, and may further comprise packaging and/or printed material. The printed material may provide information on the contents of the article, and may provide instructions or other indication of how the contents of the article may be used. In an embodiment, the printed material provides an indication of a disease or disorder that is to be treated using the contents of the article.

In embodiments, when polynucleotides are delivered, they may comprise modified polynucleotides or other modifications, such as phosphate backbone modifications, and modified nucleotides, such as nucleotide analogs. Suitable modifications and methods for making nucleic acid analogs are known in the art. Some examples include but are not limited to polynucleotides which comprise modified ribonucleotides or deoxyribonucleotides. For example, modified ribonucleotides may comprise methylations and/or substitutions of the 2′ position of the ribose moiety with an —O— lower alkyl group containing 1-6 saturated or unsaturated carbon atoms, or with an —O-aryl group having 2-6 carbon atoms, wherein such alkyl or aryl group may be unsubstituted or may be substituted, e.g., with halo, hydroxy, trifluoromethyl, cyano, nitro, acyl, acyloxy, alkoxy, carboxyl, carbalkoxyl, or amino groups; or with a hydroxy, an amino or a halo group. In embodiments modified nucleotides comprise methyl-cytidine and/or pseudo-uridine. The nucleotides may be linked by phosphodiester linkages or by a synthetic linkage, i.e., a linkage other than a phosphodiester linkage. Examples of inter-nucleoside linkages in the polynucleotide agents that can be used in the disclosure include, but are not limited to, phosphodiester, alkylphosphonate, phosphorothioate, phosphorodithioate, phosphate ester, alkylphosphonothioate, phosphoramidate, carbamate, carbonate, morpholino, phosphate triester, acetamidate, carboxymethyl ester, or combinations thereof. In embodiments, the DNA analog may be a peptide nucleic acid (PNA).

The following description and Examples are intended to illustrate but not limited the disclosure.

The description and Examples illustrate that Tn7-CRISPR-Cas elements evolved a system of guide RNA categorization to accomplish a two-pathway lifestyle. Multiple mechanisms allow functionally distinct guide RNAs for transposition, a conventional system capable of acquiring guide RNAs to new plasmid and phage targets and a second providing long-term memory for access to chromosomal sites upon entry into a new host. Guide RNAs are privatized to be recognized only by the transposon-adapted system via sequence-specialization, mismatch tolerance and selective regulation to avoid toxic self-targeting by endogenous CRISPR-Cas defense systems. This description and the Examples therefore support the foregoing approaches to engineering guide RNAs for enhanced CRISPR-Cas functionality for genome modification.

The present disclosure provides, among other aspects, bioinformatic analysis of I—F3 Tn7-CRISPR-Cas elements and reveals mechanisms that allowed the evolution of guide RNA-directed transposition involving categorization of guide RNAs. The disclosure illustrates that I—F3 Tn7-CRISPR-Cas insertion events are explained by guide RNAs encoded in CRISPR arrays within the element. A form of curation allows the I—F3 elements to maintain different classes of guide RNAs to mirror the two-pathway lifestyle found with prototypic Tn7, but with a guide-RNA-only system. Guide RNA-directed transposition into the chromosome occurs via CRISPR arrays that are under the control of a specialized transcriptional regulation system that directs pathway choice or using an atypical CRISPR repeat structure that allows the guide RNA to be private to the Tn7-CRISPR-Cas transposon, which can be exploited for genome modification, as described above. Guide RNAs encoded by the elements that recognize the chromosome also have mismatches that are tolerated for directing transposition, but not for interference by a canonical I-F1 system. The guide RNA attributes found in I—F3 Tn7-CRISPR-Cas elements help explain how they interact with related type I-F CRISPR-Cas systems, such as the ability to tolerate self-targeting guide RNAs that would otherwise cause canonical CRISPR-Cas systems to degrade the host chromosome. The disclosure takes advantage of these discoveries as described above to provide improved approaches to DNA editing, and as illustrated in the following Examples.

### Example 1

### I—F3 Tn7-CRISPR-Cas Element Targeting is Explained by Spacers in Atypical CRISPR Array Configurations

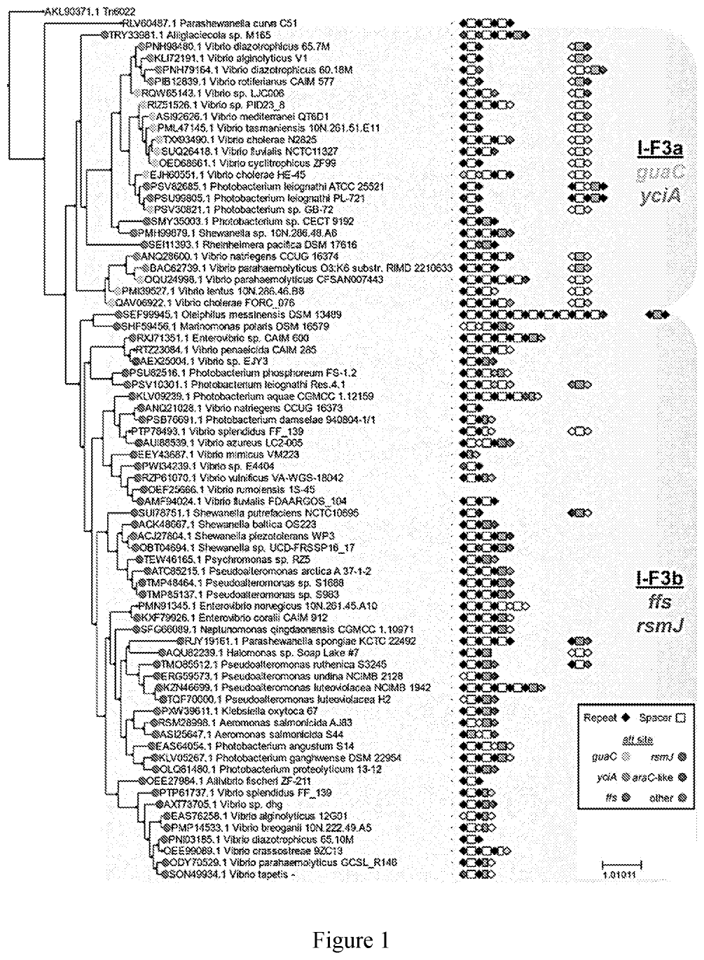

We conducted a bioinformatics analysis of the I—F3 family of Tn7-CRISPR-Cas elements. An analysis of over 53,000 genomes from gamma proteobacteria identified 802 Tn7-like elements that encode the type I—F3 CRISPR-Cas system found in two branches (FIG. 1). One branch, I-F3a, primarily uses attachment sites adjacent to the yciA and guaC (IMPDH) genes. Elements in a second branch, I-F3b, are primarily found in an attachment site downstream of the ffs gene encoding the RNA component of the signal recognition particle and a minor branch with elements residing downstream of the rsmJ gene. As part of this analysis we reexamined CRISPR arrays and made a striking finding that altered our understanding of how transposition is targeted across all of the I—F3 elements. The disclosure demonstrates, without intending to be bound by any particular theory, that the insertion position of all elements can be explained by guide RNA-directed transposition; essentially all of the I—F3 elements include a spacer within element-encoded CRISPR arrays that matches a region ˜48 bp from the right end of the element (FIGS. 1, 2a, and 2b), and as illustrated in the sequence listing. In each of these cases the spacer in the array matches the same protospacer in the yciA, guaC, ffs, or rsmJ genes (FIG. 2b). In addition to being at one of the ends of the gene to direct transposition just outside the reading frame, the spacers matching the yciA, guaC, and rsmJ genes are all found in the same reading frame register that aligns the variable wobble position of the codons with every sixth position in the guide RNA, a position known to flip out and not required to match the protospacer (Fineran et al., 2014; Jackson et al., 2014; Mulepati et al., 2014; Zhao et al., 2014). Around six percent of Tn7-CRISPR-Cas insertions identified in bacterial genomes are not located in one of the four major att sites. However, even with the insertions outside the major att sites we could still identify a spacer in the array that was specific to a protospacer ˜48 bp from the right end of the element (FIG. 1).

The spacers that recognize each one of the four major att sites are located in a specific position in the element-encoded CRISPR array(s). There are trends with this position and the configuration of the CRISPR arrays that differed in the two major branches of I—F3 elements. In the branch of I-F3a elements, the spacer that matched the yciA or guaC att sites was located after a 70-90 bp gap in the array found immediately downstream of the tniQ, cas8/5, cas7, cas6 operon (FIGS. 2a and 2c)(see below). In these cases where the CRISPR array was not contiguous it was not clear if the array was transcribed as a single pre-crRNA and/or if all of the spacers were capable of being matured into functional guide RNA complexes (addressed below). In the I-F3b branch of elements that recognizes att sites associated with the ffs and rsmJ genes the att-site specific spacer tended to be found in the single CRISPR array located downstream of the tniQ-cas operon, but always as the last spacer in the array (FIGS. 1 and 2a).

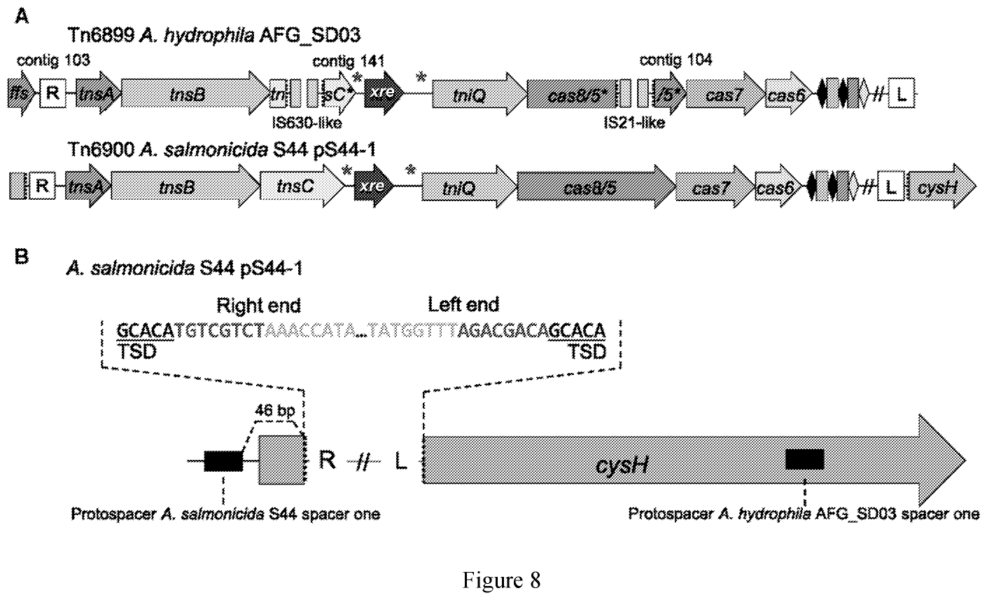

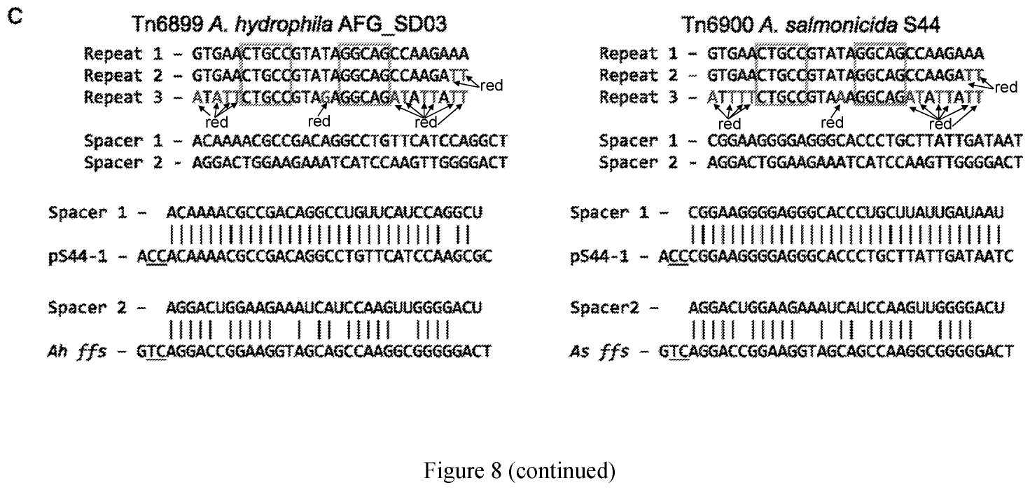

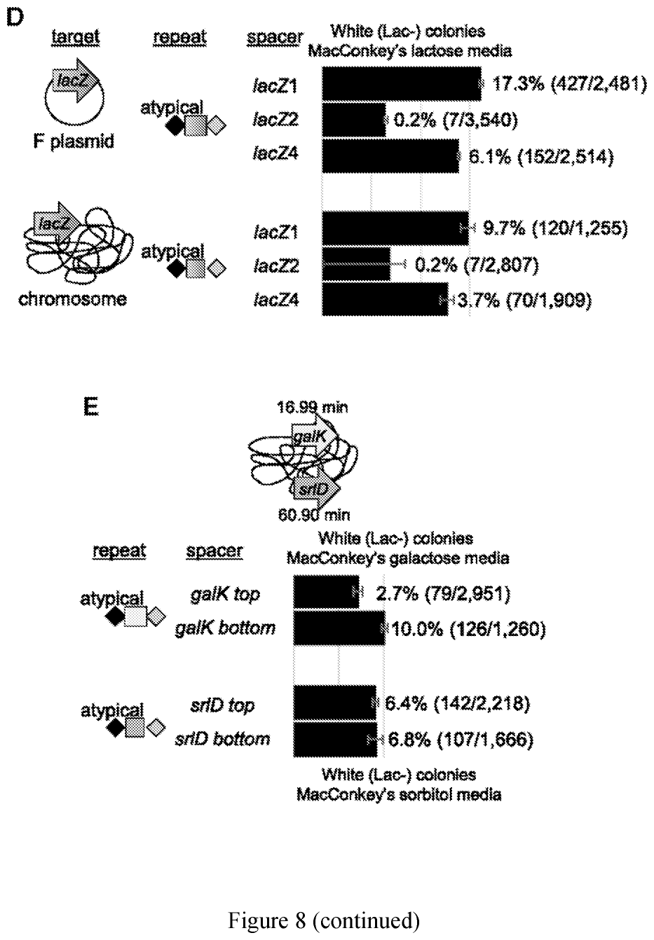

Only one transposition event was identified in a plasmid rather than in the chromosome in our analysis. The Tn7-CRISPR-Cas element in Aeromonas salmonicida S44, Tn6900, was on a large plasmid (pS44-1) that is predicted to be mobile based on the presence of genes with known roles in conjugal DNA transfer (tra genes). Transposition into the site on the plasmid could still be explained by a guide RNA encoded in the array, however, in this case the spacer was at the leader-proximal position in the array (FIG. 8a-c). Interestingly, a near-identical Tn7-CRISPR-Cas element, Tn6899, found in the ffs att site in Aeromonas hydrophila AFG_SD03 (Boehmer et al., 2018) had a spacer that recognized the same plasmid-encoded gene, but at a different position (FIG. 8b-c), suggesting a possible plasmid vector important for the dispersal of these elements within Aeromonas.

In addition to their distinct position in the CRISPR array, the att spacers were flanked by repeats with novel sequences. New spacers are added to a CRISPR array at the leader-proximal end of the array in a process that duplicates the leader-proximal repeat (Xiao et al., 2017). Therefore, although repeats can diverge over time, the first and second repeats start out identical in CRISPR arrays. In I—F3 Tn7-CRISPR-Cas elements the terminal spacer that was used for guide RNA-directed transposition into the chromosome was invariably flanked by repeats that were highly diverged from the leader-proximal repeat (FIG. 2c and the sequence listing). As discussed above, the present disclosure refers to the diverged repeats as “atypical” repeats, and a guide RNA formed from these sequences atypical guide RNAs.

### Example 2

### Highly Diverged Atypical Repeat-Spacer Units Form Functional Guide RNA Complexes

To analyze the unique nature of the CRISPR array structure found in I—F3 Tn7-CRISPR-Cas elements we established guide RNA-directed transposition in a heterologous and genetically tractable system, E. coli. Elements identified in a plasmid in Aeromonas salmonicida S44 and in the ffs attachment site in Aeromonas hydrophila AFG_SD03 were of particular interest because they were near-identical, but found in different species at distinct insertion points suggesting they were functional recently (FIG. 8a). For the transposition (Tns) and Cas proteins we utilized a coding sequence configuration we predicted to be active for transposition by looking for consensus across multiple elements found in Aeromonas.

Previous studies trying to establish Tn7-CRISPR-Cas transposition in a heterologous host relied heavily on indirect PCR-based techniques to assess transposition, techniques that are vulnerable to artifacts (Rice et al., 2020; Strecker et al., 2020). To develop a more complete picture of Tn7-CRISPR-Cas transposition, we used an assay that monitored full transposition events. A mini Tn7-CRISPR-Cas element was situated in the chromosome, the donor site for transposition in the described assays, constructed with cis-acting transposon end-sequences predicted by putative TnsB-binding sites (Peters, 2014) flanking an antibiotic resistance determinant. In this assay, candidate transposition targets resided on a conjugal F plasmid. After inducing expression of the components of the guide RNA-directed transposition system, full transposition events were detected by mating the conjugal plasmid into a tester strain and screening for the antibiotic resistance gene in the mini transposon (FIG. 9a). The tnsABC, tniQ-cas8/5, 7, 6, and the CRISPR array were expressed from three separate expression vectors.

Initially we analyzed candidate guide RNAs produced from the wild-type configuration of the CRISPR array found in Tn6900 in A. salmonicida 544. In this configuration the leader-proximal spacer was a perfect match to a mobile plasmid-encoded gene from the native host and the second/terminal spacer had a degenerate match to the ffs protospacer with 10 mismatches (FIG. 3a, 8c). Some of the mismatches between the spacer and the protospacer in the target were at every sixth position and therefore would not impact recognition of the ffs guide RNA target (FIG. 8c). Monitoring transposition following expression of the native array configuration confirmed that functional guide RNAs were produced both from the spacer with the canonical repeat structure at the leader-proximal position and the terminal spacer flanked by highly diverged atypical repeats (FIG. 3b). Interestingly, guide RNA-mediated transposition occurred at a higher frequency with the ffs-specific spacer even though it contained mismatches and was flanked by atypical repeats (FIG. 3b).

To test the individual contributions of the spacer, protospacer, and repeat sequences, we designed CRISPR array constructs with leader-proximal (typical) or terminal (atypical) flanking repeats as a single guide RNA expression construct and tested various native and synthetic spacer sequences individually. Not only were the guide RNAs with the atypical repeats functional, but also consistently allowed a higher frequency of transposition when compared to the typical repeat when tested with three different spacers (FIG. 3c). Additionally the ffs-specific spacer showed a higher frequency of transposition than the spacer directed at the plasmid target even though the plasmid spacer had a perfect match to its target and the ffs spacer had 10 mismatches to its target (several that were not at the sixth positions that are predicted to be flipped out) (FIG. 3c). Altering the native ffs-specific spacer so that it was a perfect match to the ffs protospacer consistently allowed a modestly higher frequency of transposition (FIG. 3c-d).

Guide RNA complexes were also designed using spacers matching different positions in lacZ (FIGS. 3d-e). We found that transposition frequency varied as much as 10-fold with different spacers, even though the sequences recognized all had the same candidate PAM sequence, a result that was not explained by the DNA strand that was targeted in the highly expressed lacZ gene (FIGS. 3d-e). However, regardless of the spacer tested, a modestly higher transposition frequency was consistently found with guide RNAs with the atypical repeats when compared with typical repeats in the Tn7-CRISPR-Cas system from A. salmonicida S44 (FIGS. 3c-d). These experiments confirmed that a functional guide RNA complex could be produced from atypical repeats and indicated that the functionality of these complexes show important differences from typical repeats. We found that multiple different positions could also be targeted in the E. coli chromosome using guide RNA-directed transposition supporting a view that this was not a plasmid-specific process (FIG. 8d-e).

### Example 3

Atypical Repeats Form Functionally Distinct Guide RNA Complexes with the Tn7-CRISPR-Cas System from A. salmonicida S44

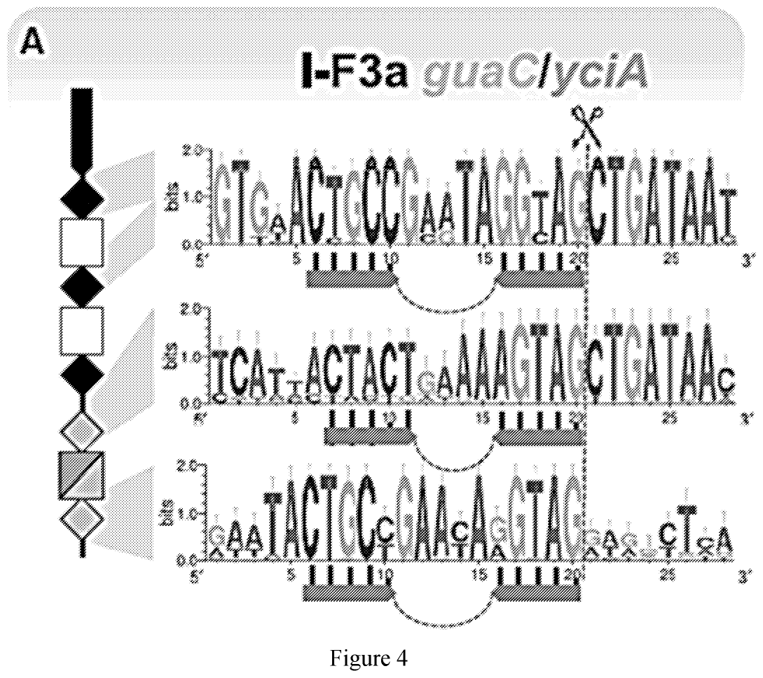

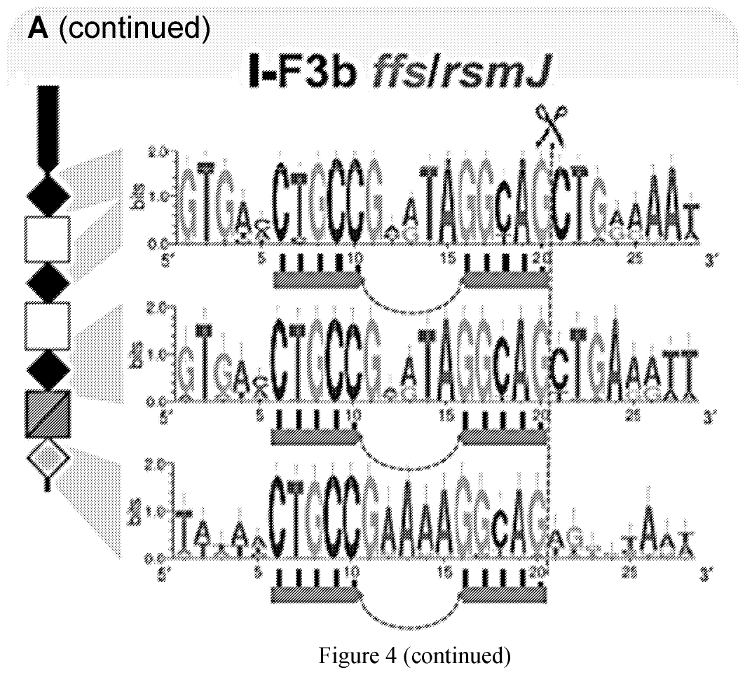

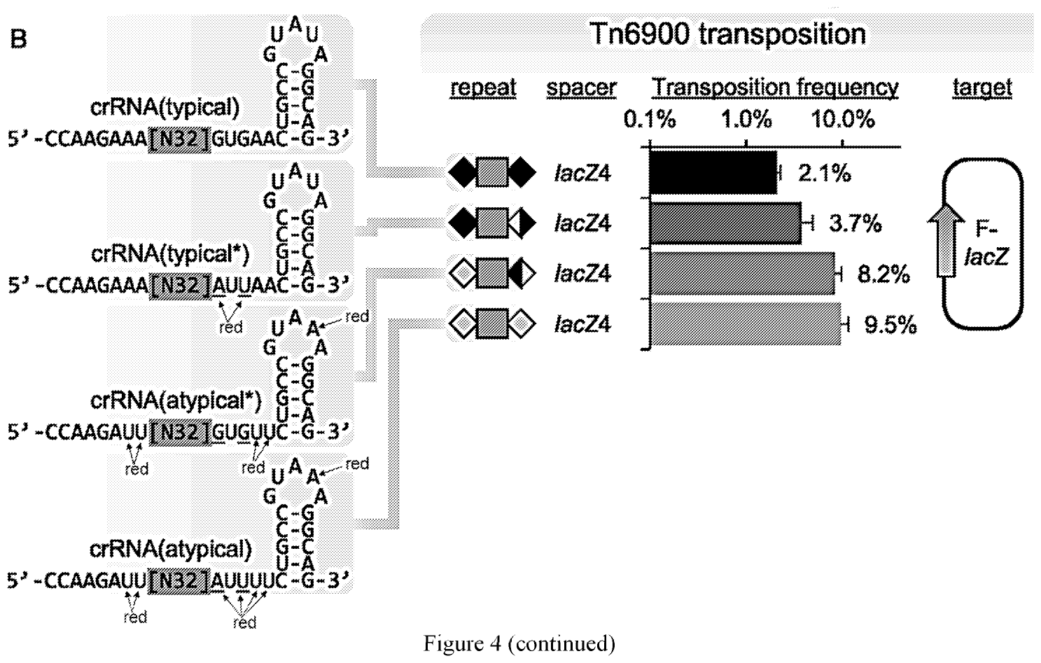

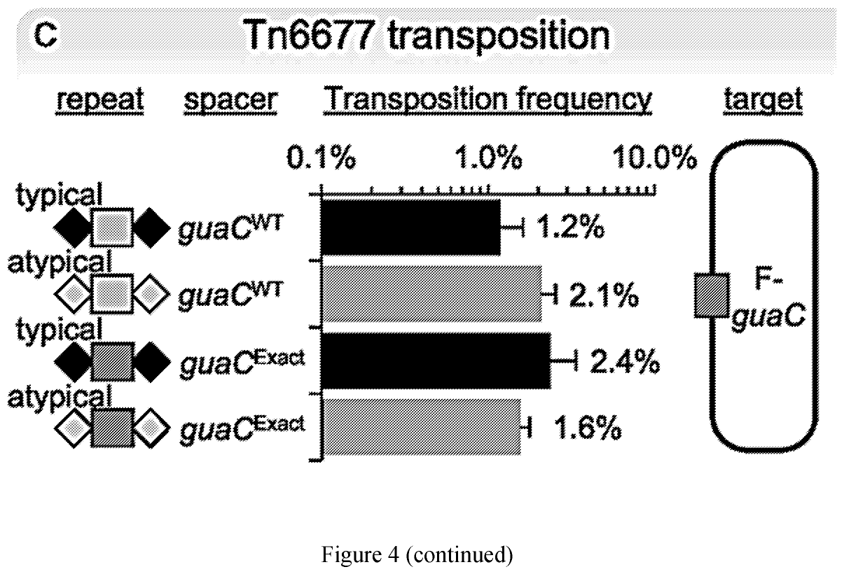

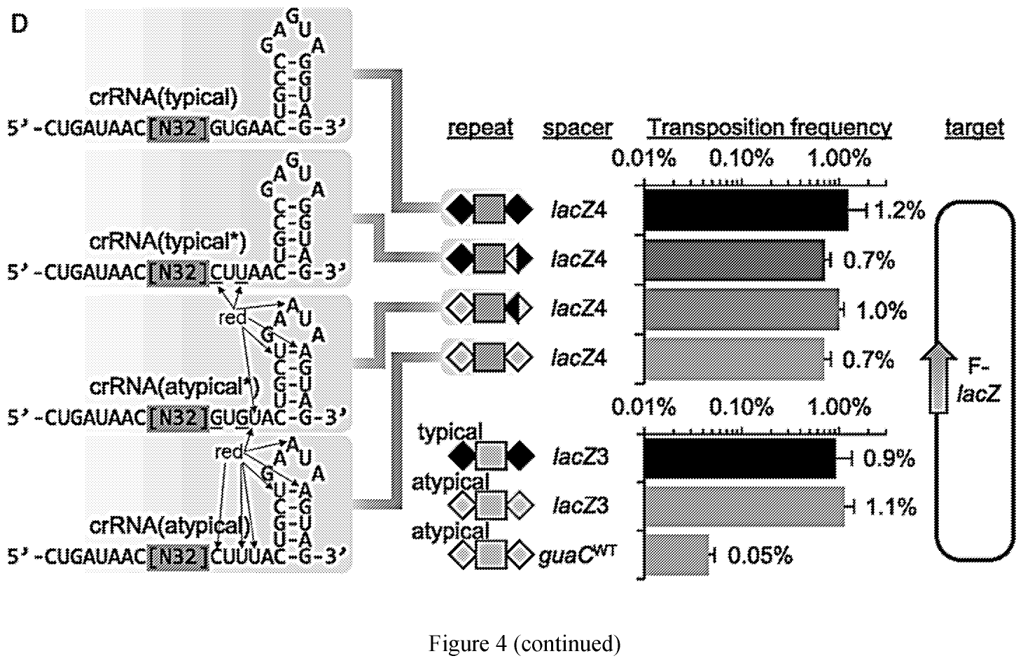

Our experiments suggested that the guide RNA produced from atypical repeats was functional and appeared to allow enhanced transposition activity with the system derived from the Tn6900 element in A. salmonicida S44. To get a better understanding of the relevance of differences in the repeat sequences, we compared the sequences of the leader-proximal typical repeats with the atypical repeats flanking the terminal spacers to look for common trends across the two branches (FIG. 4a). In both branches there were common trends in the final repeat encoding the 3′ handle of the guide RNA with a tendency to lose the typical GTG (positions 1-3), a loss of conservation of the region cleaved from the final guide RNA (positions 21-28), and a general enrichment for adenines in the loop (FIG. 4a). Functional differences from changes with the typical and atypical repeats were examined with Tn6900 by making changes to the repeat regions encoding the 5′ and 3′ handles of guide RNAs (FIG. 4b). Changing the GUG region to an AUU in the 3′ handle of a typical repeat (typical*) or changing the AUU to a GUG in the atypical repeat (atypical*) resulted in only small changes to the frequency of guide RNA-directed transposition (FIG. 4b), suggesting these conserved positions are not alone responsible for the atypical repeat frequency advantage and that a more complicated interdependency is at play.

Previous work with a different I—F3 Tn7-CRISPR-Cas systems found with the Tn6677 element from Vibrio cholerae HE-45 indicated that guide RNA complexes could be directed to programed target sites in E. coli using guide RNAs (Klompe et al., 2019). The Tn6677 element is in the I-F3a branch of elements and provided a good point of comparison for understanding differences between the two branches of I—F3 Tn7-CRISPR-Cas elements (FIG. 1). Tn6677 naturally resides in the att site downstream of guaC and consistent with the trends we identified above, this element carries the att site targeting spacer in a noncontiguous array with an atypical repeat structure (FIG. 2). The Tns, Cas, and CRISPR array modules from Tn6677 were constructed under lactose and arabinose expression systems and tested in the transposition assay used for the Tn6900 derivative above (FIG. 9a). We found that transposition into the native guaC attachment site used by Tn6677 required the guaC-specific guide RNA encoded in the array and that the atypical repeats in Tn6677 were also functional (FIG. 4c). However, unlike the Tn6900 derivative, a similar frequency of transposition was found with the typical and atypical arrays (FIG. 4c) or with modest changes in the typical and atypical repeats with the Tn6677 element (FIG. 4d). Naturally occurring Tn7-like and Tn7-CRISPR-Cas elements control the left to right orientation with which they insert. Tn6677 may be relaxed for orientation control (FIG. 9b) (Klompe et al., 2019). The Tn6900 derivative showed bias for one orientation found with canonical Tn7 and found naturally when 24 independent insertions were analyzed (FIG. 9b). Tn6900 derivative insertions were ˜48 bp from the protospacer and occurred with target site duplication (FIG. 9c).

### Example 4

Guide RNAs can be Made Private to I—F3 Transposition by Mismatch Tolerance and a Specialized Function with Atypical Guide RNAs

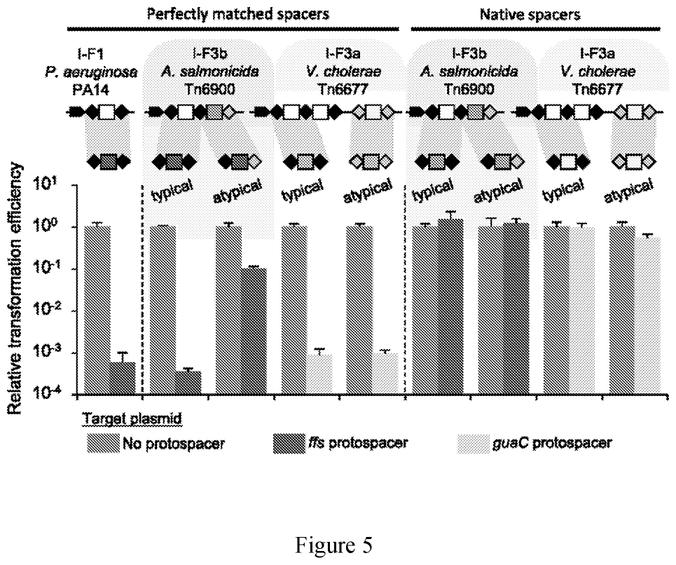

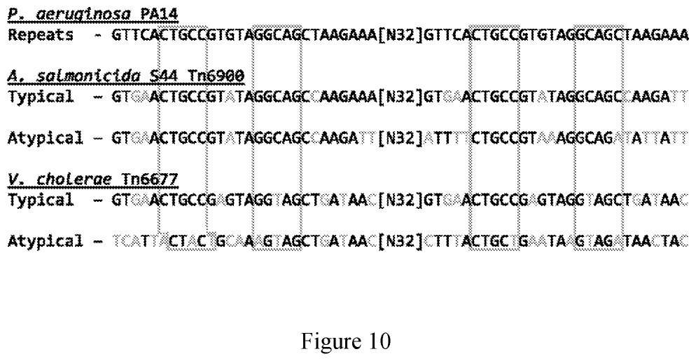

A question not previously addressed with Tn7-CRISPR-Cas systems involves possible cross-talk between CRISPR arrays with other type I-F CRISPR-Cas systems. If the CRISPR array from a Tn7-CRISPR-Cas element with the guide RNAs specific to the chromosome could be used by a standard I-F1 system, the chromosome att site would be a target for degradation. This could limit the spread of I—F3 Tn7-CRISPR-Cas elements if it entered a new host that encoded a standard I-F1 CRISPR-Cas system. We investigated if the typical and atypical guide RNAs encoded in I—F3 CRISPR arrays could be accessed by a I-F1 system (Chowdhury et al., 2017). In a P. aerigunosa system we co-expressed the Cas proteins and a single spacer CRISPR array with a T7 expression system (Vorontsova et al., 2015). Repeats from the type I-F1 system from P. aerigunosa, the type I-F3a V. cholerae Tn6677 system or the type I-F3b system derived from A. salmonicida Tn6900 were examined using a transformation efficiency assay examining plasmids with and without a protospacer. In control experiments we observed robust interference using the I-F1 CRISPR-Cas system from P. aeruginosa PA14 (FIG. 5). Transformation was decreased over three orders of magnitude with the plasmid encoding a protospacer compared to a plasmid that lacked the protospacer. Similarly, the typical repeats of the I—F3 systems from Tn6677 and Tn6900 also allowed robust interference with the plasmid transformation assay when they contained an exact match to the protospacer in the plasmid. The repeats from the canonical I-F1 and I—F3 Tn7-CRISPR-Cas systems are similar (FIG. 10) and it is likely that the I—F3 Tn7-CRISPR-Cas systems rely on standard I-F1 systems for spacer acquisition.

We also tested the tolerance for mismatches in the I-F1 interference system, based on the observation that mismatches were common in the att site guide RNAs found in the Tn7-CRISPR-Cas systems. While the native mismatches had little or no effect on the ability to function for guide RNA-directed transposition with the Tn6900 derivative with 10 mismatches (FIG. 3c) and Tn6677 element with 7 mismatches (FIG. 4c), these same guide RNAs had a profound effect on interference with the I-F1 CRISPR-Cas system from P. aeruginosa PA14, allowing no observable interference in the transformation assay (FIG. 5). This indicates a form of privatization, where mismatches with the guide RNA have minimal or no impact on guide RNA-directed transposition, they are rendered unusable by the I-F1 system tested in our work.

We also determined if the specialized atypical guide RNAs can be used by a canonical I-F1 system. When a spacer was situated with atypical repeats from the I-F3b system found with the Tn6900 derivative, guide RNA complexes formed with atypical repeats were drastically reduced in their ability to function for interference in the plasmid transformation assay, even with a perfect spacer-protospacer match (FIG. 5). The compromised use with the atypical repeats for interference was in contrast to the enhanced use we found for guide RNA-directed transposition with the I-F3b system (FIGS. 3 and 4). This result indicates a second mechanism that would allow chromosomal-targeting spacers to be tolerated in hosts with standard I-F1 CRISPR-Cas systems by allowing them to remain private to the I-F3b system. This privatization was absent in the Tn6677 I-F3a system from V. cholerae. With the I-F3a Vc system, robust interference was found with either the typical repeats or the highly diverged atypical array from this element. However, the results below suggest that I-F3a Tn7-CRISPR-Cas elements can use a separate transcription network to help tolerate self-targeting spacers.

### Example 5

### I—F3 Elements Utilize Xre-Family Transcriptional Regulators to Regulate CRISPR-Cas Components

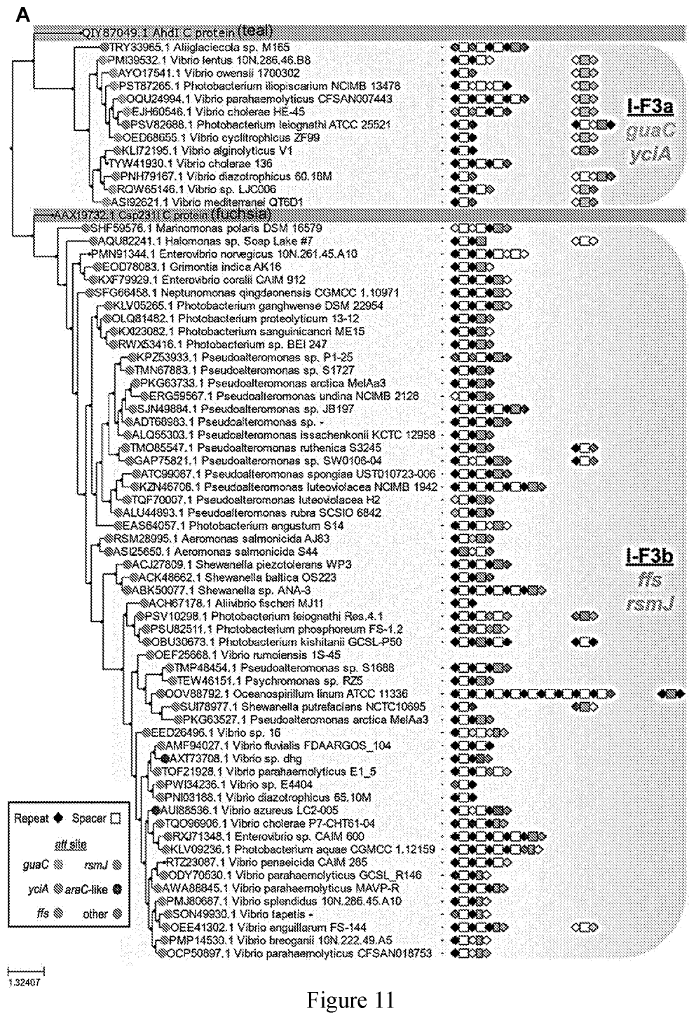

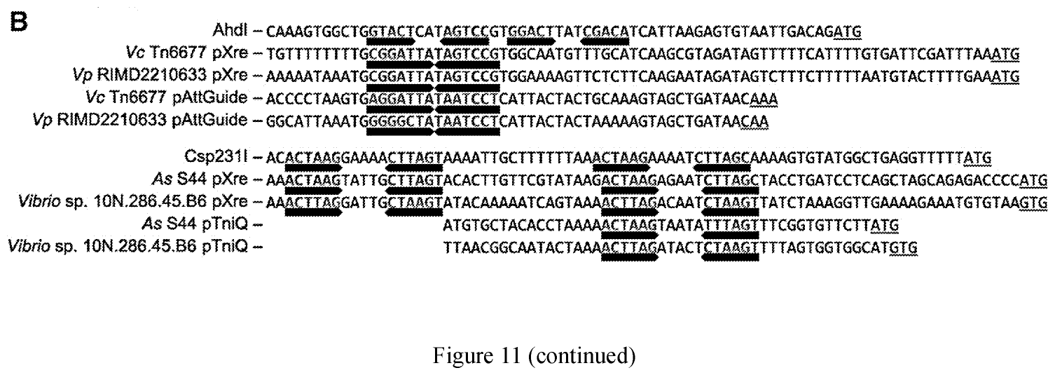

To better understand I—F3 Tn7-CRISPR-Cas element dissemination, we searched for genes conserved among diverse members of this group. One of the other genes found conserved across I—F3 Tn7-CRISPR-Cas elements were predicted Xre-family transcriptional regulators. The xre gene resides at a conserved position between the tnsABC and tniQ-cas8/5, 7,6 operons in nearly all I—F3 elements (FIG. 2a). While each of the two branches of I-F3 elements have xre genes, the predicted regulatory gene in each branch segregated with phylogenetically distinct families of controller (C) proteins associated with restriction-modification systems. I-F3a elements have a 68 amino acid Xre protein related to C.AhdI and I-F3b elements have a ˜100 amino acid Xre protein related to C.Csp231I (FIG. 11a). Candidate regulatory features could also be identified with the tniQ-cas and CRISPR arrays based on homology with the previously established systems (FIG. 11b, see below)(Streeter et al., 2004).

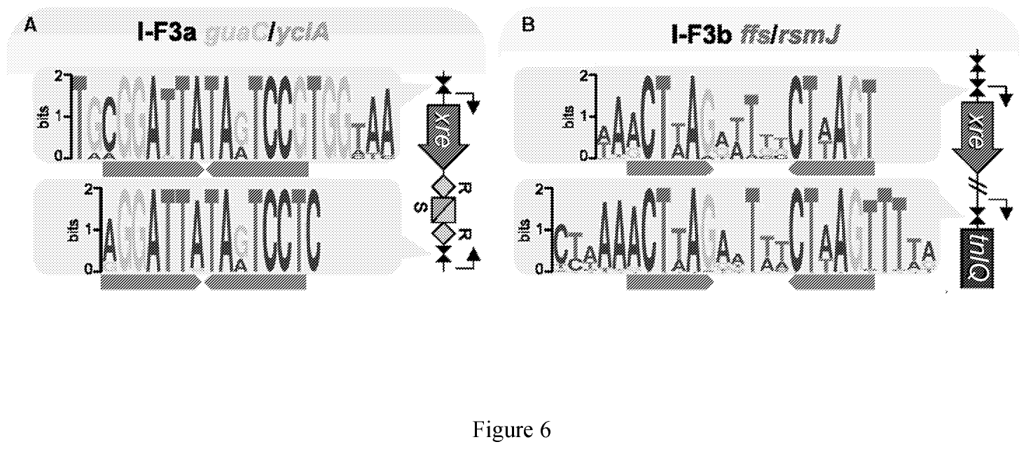

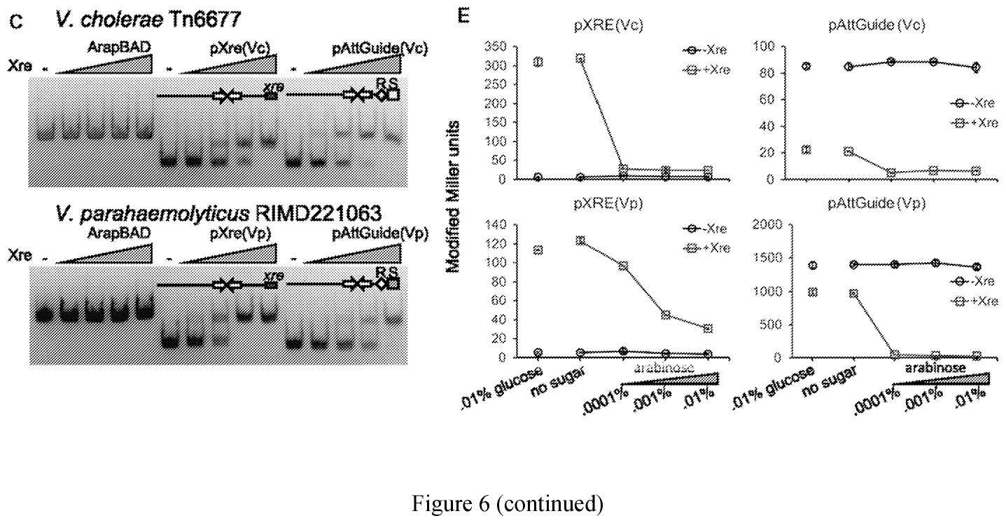

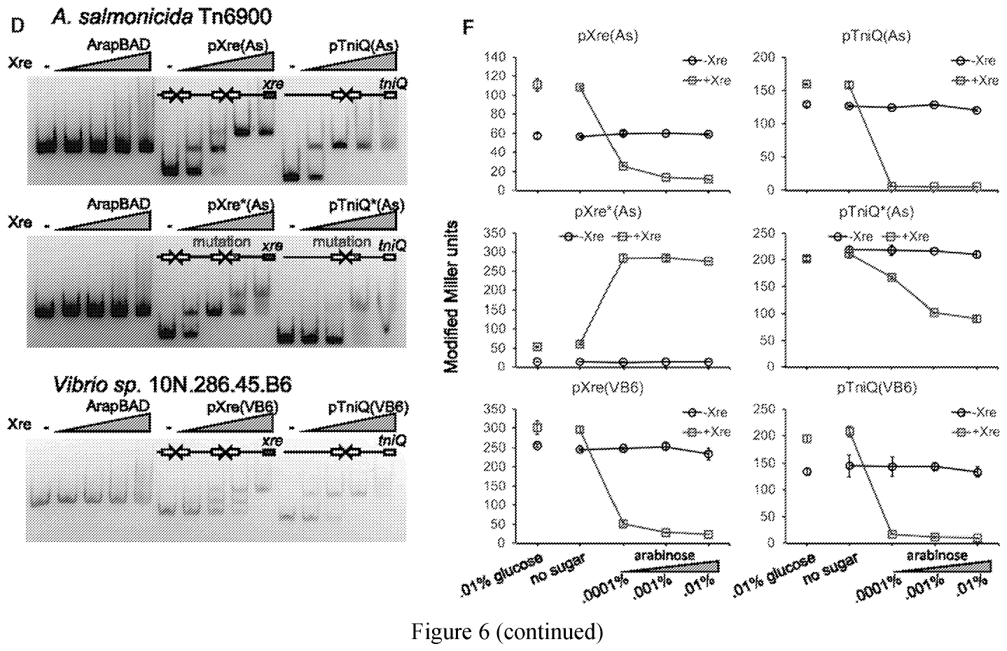

We analyzed putative promoter regions in I-F3a elements and discovered candidate sites for Xre-mediated regulation upstream of xre as well as directly upstream of the att-targeting spacer in Tn6677 and other members of this branch of elements (FIG. 6a). The regulatory regions were confirmed in vitro with two elements in the I-F3a branch, V. cholerae HE-45 Tn6677 (Vc) and V. parahaemolyticus RIMD221063 (Vp)(FIG. 6c). A functional role for this interaction was shown by a LacZ reporter assay. Xre was found to autoregulate its own pXre promoter, which allowed minimal transcription without Xre, was activated by low amounts of Xre, and repressed as the expression of Xre increased (FIG. 6e). Meanwhile the promoter identified for the att-targeting spacer (pAttGuide) was highly expressed when Xre was not present and increasingly repressed with increasing amounts of Xre induction (FIG. 6e). As shown below, this system provides a burst of the atypical guide RNA that is specific to the guaC or yciA att sites with I-F3a elements upon entry into a new host via zygotic induction.

I-F3b elements were also surveyed for inverted repeat motifs to investigate the functional role of the conserved C.Csp231I-like Xre regulator. Like I-F3a elements, conserved motifs were found in the promoter region of xre that were nearly identical to those used by C.Csp231I (FIG. 6b, FIG. 11b)(McGeehan et al., 2011). Unlike the I-F3a elements, the conserved motif could not be identified upstream of the CRISPR arrays with I-F3b elements, and instead we found a single copy of this motif upstream of the tniQ-cas8/5, 7,6 operon (FIGS. 2a and 6b). The regulatory regions were confirmed in vitro with two I-F3b elements from A. salmonicida S44 Tn6900 (As) and Vibrio sp. 10N.286.45.B6 (VB6). Binding to the two predicted motifs in the upstream region of Xre could be visualized as two separately migrating species (FIG. 6d). Mutating the xre-proximal regulatory motif weakened interaction as demonstrated by the higher concentration of protein required to achieve a full mobility shift (FIG. 6d). We additionally visualized interaction with the motif upstream of tniQ-cas8/5, 7, 6 and confirmed the sequence-specific nature of binding by utilizing a mutated motif which weakened interaction. LacZ reporter assays were again used to confirm a functional role in regulation. Xre regulator was shown to act as a repressor of its own pXre promoter (FIG. 6f). Interestingly, mutation of the proximal binding site which impaired binding in vitro resulted in Xre regulator instead acting as an activator, suggesting interaction with the distal site activates transcription while interaction with the proximal site represses it (FIG. 6f). Similar to the result with the I-F3a elements, the Xre regulator was able to repress tniQ-cas8/5, 7,6 expression and this repression was impaired by mutation of the conserved binding motif (FIG. 6f).

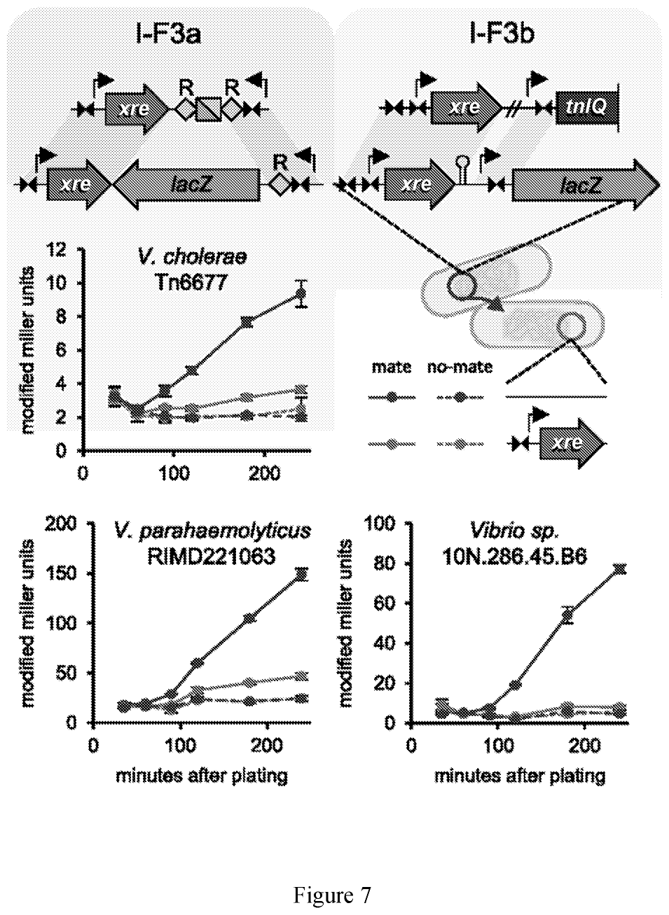

An additional assay was used to confirm zygotic induction following conjugal transfer of regulatory regions with examples from both the I-F3a and I-F3b systems. Consistent with the biochemistry and expression control data with the Xre proteins found in Tn7-CRISPR-Cas elements and previously literature with controller proteins, the Xre proteins allow tight repression in an established donor and a strong burst of expression when transmitted into a new recipient (FIG. 7). Recipient strains expressing Xre regulators are immunized from this expression burst following conjugation. To signify the Xre-dependent control demonstrated with CRISPR-Cas promoters in Tn7-CRISPR-Cas elements, the disclosure includes naming the xre genes rtaC and rtbC (RNA-guided transposon/transposition I-F3a or I-F3b controller).

### Discussion of Examples

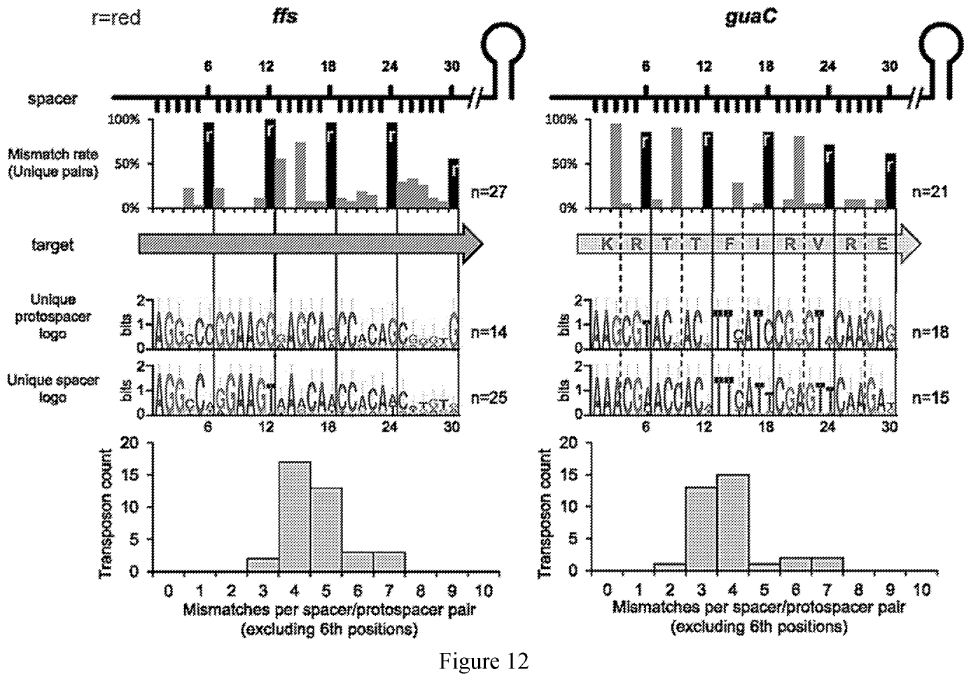

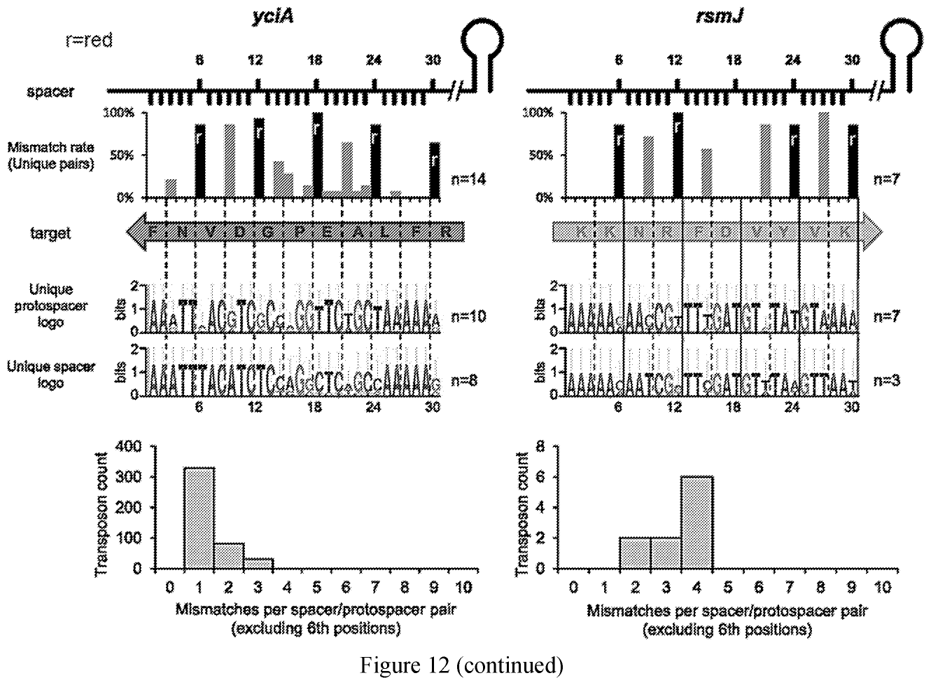

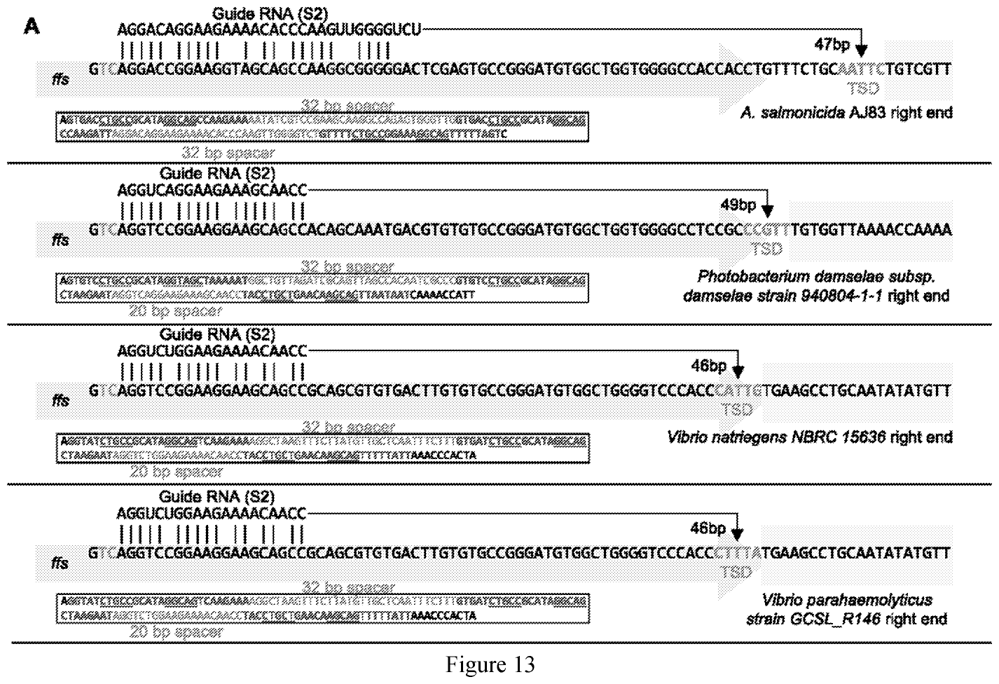

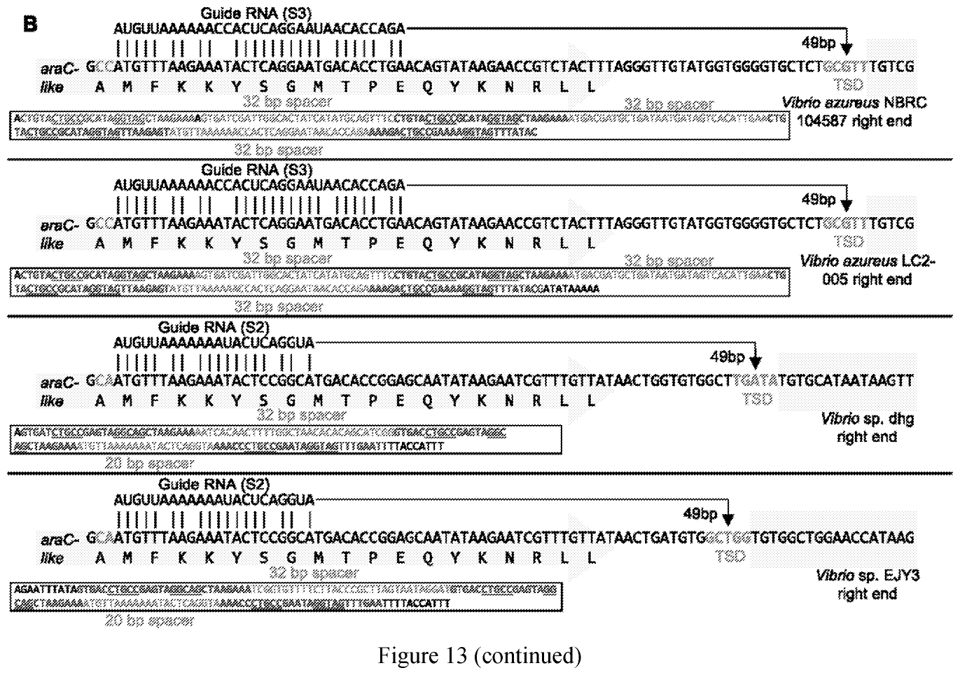

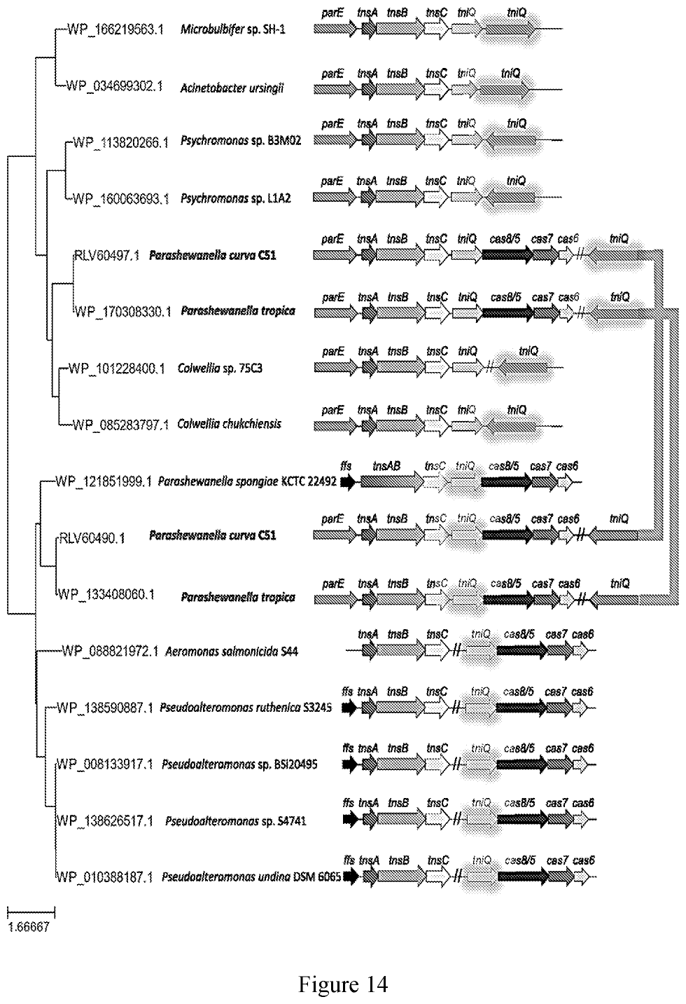

It will be recognized from the foregoing that the present disclosure demonstrates, among other items, that the spacers used to target chromosomal sites show certain characteristics; in addition to residing in the last position in the array (FIG. 1), they are flanked by highly diverged repeats (FIG. 2) and maintain mismatches that show little or no impact on guide RNA-directed transposition (FIGS. 3c and 4c), but that render them unusable for interference with a conventional 1-F1 system (FIG. 5). The repeat divergence appears to be specifically beneficial for a type I-F3b element from A. salmonicida S44, as these atypical guide RNA complexes are almost completely unusable for I-F1-mediated interference even when perfectly matched to a target (FIG. 5), while allowing for a higher level of transposition than found with the typical guide RNAs (FIG. 3). The transposon-encoded guide RNAs that allow long-term memory to direct transposition into chromosomal sites are therefore privatized to the transposon-adapted I—F3 system using mismatch tolerance, specialized atypical guide RNAs, and selective regulation to guard against toxic self-targeting by canonical CRISPR-Cas defense systems. Guide RNAs that target protein coding genes show a concentration for mismatches at the 3^{rd} positions coincident with the wobble positions (FIG. 12). In the case of the I-F3b system from A. salmonicida S44, the atypical repeat appears to be a specific adaptation that allows a higher frequency of guide RNA targeted transposition (FIGS. 3 and 4) and privatization from a canonical I-F1 interference system (FIG. 5). The type I-F3a Tn7-CRISPR-Cas system from Tn6677 did not show enhanced transposition with the atypical array found in this system; the frequency of transposition was the same with the typical and the highly diverged atypical repeat (FIGS. 4c and 4d). The disclosure demonstrates that one subbranch within the I-F3b elements the final spacer is truncated by 10 to 12 base pairs in length (FIG. 1 and FIG. 13). These smaller spacers produce functional guide RNAs as predicted by the commensurate natural repositioning of the insertion closer to the protospacer (FIG. 13). As noted above, previous work in closely related CRISPR-Cas systems suggests that guide RNAs of this length will not be functional for targeting transposition nor robust interference (Klompe et al., 2019; Kuznedelov et al., 2016). However, the ability of the I-F3b systems to accommodate shorter guide RNAs could provide another mechanism of privatization from other I-F CRISPR-Cas systems. Naturally occurring minimal type I-F2 CRISPR-Cas systems tested in the laboratory are not functional for interference with similarly truncated guide RNAs, but can still form complexes capable of forming R-loops to matching protospacers (Gleditzsch et al., 2016).

The following Materials and Method were used to produce the results described in the foregoing Examples.

### Experimental Model and Subject Details

Escherichia co/i strains were grown at 30 or 37° C. in lysogeny broth (LB) or on LB agar (unless stated otherwise in the Method Details) supplemented with the following concentrations of antibiotics when appropriate: 100 μg/mL carbenicillin, 10 μg/mL gentamicin, 30 μg/mL chloramphenicol, 8 μg/mL tetracycline, 50 μg/mL kanamycin, 100 g/mL spectinomycin.

### Method Details

### Identifying Type I-F CRISPR-Guided Tn7-Like Transposons

In total, there were 53,079 genomes were analyzed. Profile HMMs associated with TnsA (PF08722,PF08721), TnsB (PF00665), TnsC (PF11426,PF05621), TniQ(PF06527), Cas5f(PF09614), Cas6f(PF09618), Cas7f(PF09615) and Xre family proteins(PF01381), which can be downloaded from The European Bioinformatics Institute (EMBL-EBI) Pfam database, were used for detecting homologs with hmmsearch (HMMER3).

Candidate proteins were grouped into tnsABC operons and tniQ-cas operon based on their orientation and proximity. Then each tnsABC operon was grouped with its downstream tniQ-cas operon into one transposon functional unit. The Xre/HTH (helix turn helix) proteins situated between the two operons and are homologous to restriction controller proteins (blastp, identity >40%) were defined as candidate regulators.

### CRISPR Array Detection

Manually curated CRISPR repeats of Tn7-CRISPR-Cas elements were used to create a DNA sequence profile, which was used as a query for nhmmscan searches (HMMER3) to find CRISPR repeats in the downstream 20-kb region of cas6. Putative repeats were grouped into arrays by their distances to each other. The distance between repeats was required to be >55 bp and <65 bp, the bit-score threshold is −1. The distance between last repeat and previous repeat was allowed to be between 43 bp and 55 bp, but in such cases its bit-score had to be >=0.3. The sum of bit-scores of repeats in an array cannot be lower than 6.0. The longest non-overlapping arrays are collected as putative CRISPR arrays. All repeats besides the final repeat from the first array downstream of cas6 were used to create an updated repeat profile, and the CRISPR detection procedure was repeated with the new profile twice.

### Protospacer Detection

To detect protospacers that match the transposon-associated CRISPR spacers, each spacer was converted into position-specific scoring matrix (PSSM) and used to search upstream 1-kb DNA of tnsA for matches with Biopython (threshold=11.0). Because every 6th base of spacers is flipped out in type I CRISPR Cascade complex, all 6th positions of the matrix are set to have equal weight on all four bases.

Except for ffs (SRP-RNA), the major attachment site genes that containing the candidate protospacers are classified with the annotations provided in NCBI. The attachment site SRP-RNA gene (ffs) is often poorly annotated, so it was reannotated using cmsearch (Infernal) and SRP-RNA profile (RF00169) available on RFAM (//rfam.xfam.org/).

### Constructing Similarity Trees

The TnsA, TniQ and Xre proteins were clustered using Cd-hit with identity threshold set to 90%. Multiple alignments of the representatives were done with MUSCLE. Similarity trees were made with FastTree using WAG evolutionary model and the discrete gamma model with 20 rate categories as previously described (Peters et al., 2017). The visualization of the trees, major attachment sites, CRISPR arrays and matched spacers was done with ETEToolkit.

### Identifying Shared Promoter Motifs of Xre and CRISPR-Cas Genes

The transposons were classified into two groups based on associated xre lengths (68 a.a. for I-F3a or ˜100 a.a. for I-F3b) and similarities to C.AhdI and C.Csp231I. For each group, the 100 bp upstream of xre, second CRISPR array, and tniQ-cas operon were collected and deduplicated with dedupe.sh (BBTools) with threshold of 70% identity or 30 edit distance. The sequences were then sent to MEME for motif detection and comparison.

### Comparing Consensus CRISPR Repeat Sequences of Chromosome Targeting Spacers to Those of Other Spacers

To make consensus sequences of CRISPR repeats, the transposon representatives with non-redundant TniQ were selected with Cd-hit and separated into two groups based on their attachment sites being ffs/rsmJ or guaC/yciA. The upstream and downstream CRISPR repeats of the chromosome targeting spacers, and repeats not flanking chromosome targeting spacers were collected and sequence logos created using WebLogo 3.

### Transposition Assays

All transposition assays were performed in MTP1191, or one of MTP997 or MTP1196 with an F plasmid derivative.

For Tn6900 transposition, strains used to monitor transposition were made competent by standard chemical methods (Peters, 2007) and transformed with pMTP130, pMTP140, and a derivative of pMTP150, pMTP160, pMTP170, or pMTP190 onto LB agar supplemented with 100 μg/mL carbenicillin, 10 μg/mL gentamicin, 30 μg/mL chloramphenicol, and 0.2% w/v glucose. After 16 hours incubation at 37° C., several hundred transformants were washed up in M9 minimal media (Peters, 2007) supplemented with 0.2% w/v maltose and diluted to a calculated OD=0.2 in M9 supplemented with 100 μg/mL carbenicillin, 10 μg/mL gentamicin, 30 μg/mL chloramphenicol, 0.2% w/v arabinose, and 100 μM IPTG to induce transposition.

For experiments monitoring transposition frequency through loss of sugar metabolism on MacConkey's media, induction pools were incubated for 24 hours with shaking at 30° C. before being serially diluted in LB and plated on MacConkeys 1% w/v lactose, sorbitol, or galactose. Plates were incubated at 37° C. for 16 hours before colonies were counted.

For experiments monitoring transposition frequency by the mate out assay (FIG. 9a), after 24 hours incubation with shaking at 30° C., a portion of induced cultures were washed once and resuspended in LB supplemented with 0.2% w/v glucose. After 2 hours incubation at 37° C. induced pools were mixed with prepared mid-log CW51 recipient strain at a ratio of 1:5 donor:recipient and incubated with gentle agitation for 90 minutes at 37° C. to allow mating. After incubation cultures were vortexed, placed on ice, then serially diluted in LB 0.2% w/v glucose and plated on LB supplemented with 20 μg/mL nalidixic acid, 100 μg/mL rifampicin, 100 μg/mL spectinomycin, 50 μg/mL X-gal, with or without 50 μg/mL kanamycin to sample the entire transconjugant population or select for transposition respectively. Plates were incubated at 37° C. for 36 hours before colonies were counted.

Tn6677 transposition assays were performed as above with function plasmids pMTP230, pMTP240, and a derivative of pMTP250, pMTP260, or pMTP270 with the exception of 8 μg/mL tetracycline replacing gentamicin when present.

In all experiments, non-target controls where the spacer did not match the target F plasmid were used, with transposition frequency similar to non-target rate in FIG. 3B for A. salmonicida S44 transposition, or FIG. 4D for Tn6677 transposition.

### Screen Orientation of Transposition Events

Individually isolated CW51 transconjugants with mini-element insertions from the mate out assay were purified on LB supplemented with 20 μg/mL nalidixic acid, 100 μg/mL rifampicin, 100 μg/mL spectinomycin, 50 μg/mL X-gal, and 50 μg/mL kanamycin. Colony PCR was performed using primer set A (JEP1386+JEP1958) or primer set B (JEP1387+JEP1958) to capture position and orientation of insertion events.

P. aeruginosa CRISPR Interference Assays

All interference assays were performed in BL21-AI. BL21-AI made competent by standard chemical methods (Peters, 2007) and transformed with pOPO322, pCsy_complex, and a derivative of pCOLADuet-1 onto LB agar supplemented with 100 μg/mL carbenicillin, 100 μg/mL spectinomycin, 30μg/mL chloramphenicol, and 0.2% w/v glucose. Overnight cultures grown in LB agar supplemented with 100 μg/mL carbenicillin, 100 μg/mL spectinomycin, 30 μg/mL chloramphenicol were diluted 1:50 in LB supplemented with 100 g/mL carbenicillin, 100 μg/mL spectinomycin, 30 μg/mL chloramphenicol, 100 μM IPTG and 1 mM arabinose. Cultures were grown to OD=0.4 before electrocompetent cells were prepared by standard methods (Peters, 2007) and transformed with 1 ng pOPO275 or pOPO390. Cells were recovered in SOC at 37° C. for one hour before being serially diluted and plated on LB supplemented with 100 μg/mL carbenicillin, 50 μg/mL kanamycin, 30 g/mL chloramphenicol, and 100 μg/mL spectinomycin. Plates were incubated at 37° C. for 16 hours before colonies were counted.

### Xre Protein Purification

pOPO223, pOPO239, pOPO331 or pOPO360 were transformed into BL21 (DE3), which was cultured in Terrific Broth at 37° C. and induced with 0.1 mM IPTG during log-phase. Cells were cultured an additional 12-16 hours at 18° C. before being collected with centrifugation and lysed by sonication in nickel buffer (20 mM HEPES-NaOH (pH 7.5), 500 mM NaCl, 30 mM imidazole, 5% (v/v) glycerol, 5 mM β-mercaptoethanol) supplemented with 0.15 mg/mL lysozyme. Lysate was cleared by centrifugation and loaded on Nickel-NTA column, washed with nickel buffer, and eluted over a 30 mM to 500 mM imidazole gradient in nickel buffer. Selected purified fractions were pooled, dialyzed and buffer exchanged into storage buffer (20 mM HEPES-NaOH (pH 7.5), 100 mM KCl, 5% (v/v) glycerol, 1 mM DTT). The purified proteins were snap-frozen with liquid nitrogen and stored at −80° C.

### Electrophoretic Mobility Shift Assay (EMSA)

The promoter fragments of putative Xre regulated genes and their mutated variants were PCR amplified and purified. 100 nM DNA was incubated with different amounts of purified Xre proteins in equilibrium buffer (50 mM Tris-HCl (pH 8.0), 1 mM DTT, 10 mM MgCl2) at 25° C. for 20 minutes then mixed with glycerol (final concentration 6%). EMSAs were performed in 6% non-denaturing TBE PAGE (Polyacrylamide gel) with 0.5×TBE as running buffer, running at 80V for one hour at room temperature. The gels were EtBr stained and visualized with UV imager.

DNA substrates were produced as follows: ArapBAD was amplified from pBAD24 (JEP175+JEP1364), pXre(Vp) and pAttguide(Vp) were amplified from V. parahaemolyticus RIMD221063 (JEP1956+JEP1957, pXre(Vp); JEP1954+JEP1955, pAttguide(Vp)), pXre(Vc) and pAttguide(Vc) were amplified from gBlock11 (JEP29+JEP30, pXre(Vc); JEP1553+JEP82, pAttguide(Vc)), pXre(As) was amplified from pOPO08 (JEP1321+JEP81), pTniQ(As) was amplified from pOPO09 (JEP1322+JEP81), pXre*(As) was amplified from pOPO10 (JEP1321+JEP81), pTniQ*(As) was amplified from pOPO11 (JEP1322+JEP81), pXre(VB6) was amplified from pOPO06 (JEP1553+JEP81), and pTniQ(VB6) was amplified from pOPO07 (JEP1554+JEP81).

### In Vivo Promoter Assay

pOPO256, pOPO258, pOPO364, or pOPO345 and a derivative of pOPO221 were transformed into BW27783 made competent by standard chemical methods (Peters, 2007) on LB agar supplemented with 100 μg/mL carbenicillin and 30 μg/mL chloramphenicol. Overnight cultures grown in LB supplemented with 100 μg/mL carbenicillin and 30 μg/mL chloramphenicol were diluted 1:100 into LB supplemented with 100 μg/mL carbenicillin, 30 g/mL chloramphenicol and various concentrations of glucose or arabinose as indicated in FIG. 6 and cultured for an additional 20 hours at 30° C. The LacZ activities were measured with standard Miller unit assay (Malke, 1993).

### Zygotic Induction Assay

P0429 was made competent by standard chemical methods (Peters, 2007) and transformed with one of pOPO392, pOPO394, or pOPO435 on LB agar supplemented with 50 μg/mL kanamycin to produce donor strains. DH5α was made competent by standard chemical methods (Peters, 2007) and transformed with pETDuet-1, pOPO395, pOPO397 or pOPO438 on LB agar supplemented with 100 μg/mL carbenicillin to produce recipient strains. Overnight cultures of donors and recipients grown in LB supplemented with appropriate antibiotics were diluted 1:10 in the same media and grown for two hours, then washed with LB three times to remove antibiotics. Donors and recipient strains were mixed at a 1:2 ratio and spotted on LB agar for mating at 37° C. The LacZ activity of the mating cells at different time points were measured with standard Miller unit assay (Malke, 1993). The non-mating control was done by spotting donors and recipients separately on the same plate.

### Strain Construction

MTP997 and MTP1196 were constructed by transforming pMTP112 or pMTP113 into BW27783 made competent by standard chemical methods (Peters, 2007) on LB agar supplemented with 100 μg/mL carbenicillin grown at 30° C. Individual colonies were purified on LB agar supplemented with 50 μg/mL kanamycin grown at 42° C. to select for miniTn7 insertion into the chromosome while curing pMS26 derivatives. Individual colonies were purified at 30° C. on LB agar supplemented with carbenicillin or kanamycin to confirm loss of carbenicillin resistance.

MTP1191 was constructed by P1 transduction of MTP997 with bacteriophage grown on strain EMG2 to replace lacZ deletion with wild-type lac operon. Transductants were selected on M9 minimal media supplemented with 0.2% w/v lactose.

P0429 was constructed by using recombineering (Datsenko and Wanner, 2000) to replace wild-type lacZ with a lacZ::miniTn7(genR) allele PCR amplified from a miniTn7(genR) lacZ insertion library.

### Plasmid Construction

Standard molecular cloning techniques were used to make the vectors described below using vendor instructions.

pMTP112 was constructed by ligating gBlock1 into the NotI site of pMS26 following digestion with NotI. The clone used has A. salmonicida left end proximal to the Tn7 right end. pMTP113 was constructed by assembling two PCR products amplified from pSL0527 (pDonor) (JEP1858+JEP1859 and JEP1860+JEP1861), one PCR product amplified from gBlock1 (JEP1862+JEP1863) and pMS26 digested with NotI using NEBuilder Hifi (NEB). pMTP114 was constructed by assembling two PCR products amplified from F plasmid (JEP1398+1340 and JEP1341+1399, GenBank: AP001918.1), one PCR product amplified from pMTP150 (JEP1343+JEP1344), one PCR product amplified from pBAD322S (JEP1345+JEP1346, GenBank: DQ131584.1) and pTSC29 digested with EcoRV using NEBuilder Hifi. pMTP115 was constructed by inserting a PCR product amplified from EMG2 (JEP1663+JEP1664, GenBank: U00096.3) into pMTP114 following digestion with BsaI using golden gate cloning (Engler et al., 2008). pMTP116 was constructed by inserting annealed oligos (JEP1485+JEP1486) into pMTP114 following digestion with BsaI using golden gate cloning. pMTP117 was constructed by inserting annealed and extended oligos (JEP1481+JEP1482) into pMP114 following digestion with BsaI using golden gate cloning. pMTP118 was constructed by inserting annealed and extended oligos (JEP1878+JEP1879) into pMTP114 following digestion with BsaI using golden gate cloning. pMTP130 was constructed by assembling gBlock2, gBlock3 and a PCR product amplified from pTA106 (JEP1146+JEP1467) digested by DraII with 3,800 bp fragment gel purified using NEBuilder Hifi. pMTP140 was constructed by assembling gBlock4, gBlock5, gBlock6 and pBAD322G digested with NcoI and HindIII using NEBuilder Hifi. pMTP150 was constructed by assembling two PCR products amplified from pBAD33 (JEP1766+JEP1767 and JEP1768+JEP1769) with gBlock7 and gBlock8 using NEBuilder Hifi.pMTP151 was constructed by inserting annealed oligos (JEP1477+JEP1478) into pMTP150 following digestion with BsaI using golden gate cloning. pMTP160 was constructed by assembling two PCR products amplified from pBAD33 (JEP1766+JEP1767 and JEP1768+JEP1769) with gBlock7 and one PCR product amplified from gBlock8 (JEP1475+JEP1773) using NEBuilder Hifi. pMTP161-165 were constructed by ligating annealed oligos (JEP1477+JEP1478, pMTP161; JEP1776+JEP1777, pMTP162; JEP1778+JEP1779, pMTP163; JEP1669+JEP1670, pMTP164; JEP1671+JEP1672, pMTP165). pMTP170 was constructed by assembling two PCR products amplified from pBAD33 (JEP1766+JEP1767 and JEP1770+JEP1769) with one PCR product amplified from gBlock7 (JEP1774+JEP1474) and one PCR product amplified from gBlock8 (JEP1475+JEP1775) using NEBuilder Hifi. pMTP171-183 were constructed by inserting annealed oligos (JEP1784+JEP1785, pMTP171; JEP1780+1781, pMTP172; JEP1782+JEP1783, pMTP173; JEP1794+JEP1795, pMTP174; JEP1796+JEP1797, pMTP175; JEP1786+JEP1787, pMTP176; JEP1788+JEP1789, pMTP177; JEP1798+JEP1799, pMTP178; JEP1800+JEP1801, pMTP179; JEP1808+JEP1809, pMTP180; JEP1810+JEP1811, pMTP181; JEP1816+JEP1817, pMTP182; JEP1818+JEP1819, pMTP183) into pMTP170 following digestion with BsaI using golden gate cloning. pMTP190 was constructed by assembling two PCR product amplified from pBAD33 (JEP1766+JEP1767 and JEP1771+JEP1769) using NEBuilder Hifi.pMTP191 and pMTP192 were constructed by annealing four oligos (JEP1928, JEP1929, JEP1930, JEP1931: pMTP191; JEP1932, JEP1933, JEP1934, JEP1935: pMTP192) and ligating with pMTP190 digested with XmaI and BsaI.

pMTP230 was constructed by assembling one PCR product amplified from pBAD33 (JEP1864+JEP1865), one PCR product amplified from pMTP130 (JEP1866+JEP1867) and pSL0284 digested with NcoI and PflFI with 3,707 bp fragment gel purified using NEBuilder Hifi. pMTP240 was constructed by assembling a PCR product amplified from pBAD322 (JEP1868+JEP1869) with pSL0284 digested with NdeI and BglI with 5,152 bp fragment gel purified using NEBuilder Hifi. pMTP250 was constructed by assembling a PCR product amplified from pCDFDuet-1 (JEP1838+JEP1839), a PCR product amplified from pBAD322 (JEP1834+JEP1835) and a PCR product amplified from pBBR1MCS-3 (JEP1836+JEP1837) using NEBuilder Hifi. pMTP260 and pMTP270 were constructed by annealing four oligos (JEP1870, JEP1871, JEP1872, JEP1873: pMTP260; JEP1908, JEP1909, JEP1910, JEP1911: pMTP270) and ligating with pMTP250 digested with XmaI and BsaI. pMTP261-264 were constructed by inserting annealed oligos (JEP1914+JEP1915, pMTP161; JEP1912+JEP1913, pMTP162; JEP1880+JEP1881, pMTP163; JEP1882+JEP1883, pMTP164) into pMTP260 following digestion with BsaI using golden gate cloning. pMTP271-274 were constructed by inserting annealed oligos (JEP1914+JEP1919, pMTP271; JEP1912+JEP1917, pMTP272; JEP1880+JEP1916, pMTP273; JEP1882+JEP1917, pMTP27) into pMTP270 following digestion with BsaI using golden gate cloning. pMTP275 and pMTP276 were constructed by annealing four oligos (JEP1920, JEP1921, JEP1922, JEP1923: pMTP275; JEP1924, JEP1925, JEP1926, JEP1927: pMTP276) and ligating with pMTP250 digested with XmaI and BsaI.

All F derivatives were made by using recombineering (Datsenko and Wanner, 2000) to replace a large region of plasmid F from strain EMG2 (GenBank: AP001918.1) with PCR fragments amplified from pMTP114 derivatives (JEP1376+1386. pMTP115, FΔ(finO-fxsA)::lacZ specR; pMTP116, FΔ(finO-fxsA)::cysH^{As} specR; pMTP117, FΔ(finO-fxsA):ffs^{As} specR; pMTP118, FΔ(finO-fxsA)::guaC^{Vc} specR).

pOPO256 was constructed by ligating a PCR product amplified from gBlock9 (JEP1657+JEP1757) digested with NdeI and HindIII into pBAD33 digested with the same enzymes. The resulting construct was digested with NdeI and XbaI and ligated with phosphorylated annealed oligos (JEP1842+JEP1843). pOPO258 was constructed by assembling a PCR product amplified from gBlock10 (JEP1764+JEP1765) with pBAD33 digested with NdeI and HindIII using NEBuilder Hifi. The resulting construct was digested with NdeI and XbaI and ligated with phosphorylated annealed oligos (JEP1842+JEP1843). pOPO364 was constructed by ligating a PCR product amplified from gDNA of V. parahaemolyticus RIMD221063 (kindly provided by Tobias Doerr) (JEP1952+JEP1960) digested with NdeI and HindIII and phosphorylated annealed oligos (JEP1842+JEP1843) into pBAD33 digested with NdeI and HindIII. pOPO345 was constructed by ligating a PCR product amplified from gBlock11 (JEP1555+JEP1556) digested with SpeI and HindIII into pBAD33 digested with XbaI and HindIII. pOPO221 was constructed by ligating a PCR product amplified from pBAD24 (JEP1759+JEP1760) digested with BsaI and XhoI with a PCR product amplified from EMG2 (JEP1761+JEP1762) digested with the same enzymes. pOPO227-230, pOPO332, pOPO334, pOPO341, and pOPO337 were constructed by ligating fragments from gBlock10 or gBlock11 digested with XhoI and StuI (gBlock10. pOPO227-230; gBlock11: pOPO332, pOPO334, pOPO341, pOPO337) into pOPO221 digested with XhoI and SmaI. pOPO329 and pOPO330 were constructed by ligating PCR products amplified from gDNA of V. parahaemolyticus RIMD221063 (JEP1956+JEP1957, pOPO329; JEP1954+JEP1955, pOPO330) digested with XhoI and StuI into pOPO221 digested with XhoI and SmaI. pOPO223, pOPO239, pOPO331, pOPO360 were constructed by ligating PCR products amplified (from gBlock9, JEP1675+JEP1758, pMTP016; from gBlock10, JEP1556+JEP1764, pMTP017; from gDNA of V. parahaemolyticus RIMD221063, JEP1952+JEP1953, pMTP018; from gBlock11, JEP1950+1951, pMTP019) digested with NdeI and XhoI into pET22b(+) digested with the same enzymes. pOPO390 and pOPO275 was constructed by ligating annealed oligos (JEP2119, JEP2120, JEP1906+JEP1907 respectively) into a PCR product amplified from pCOLADuet-1 (JEP1902+JEP1903) digested with SapI. pOPO322 was constructed by assembling a PCR product amplified from pCas1_pCas2/3 (JEP1889+JEP1890) and pACYCDuet-1 digested with NcoI and AvrII using NEBuilder Hifi. pOPO392 was constructed by assembling PCR products amplified from gDNA of V. parahaemolyticus RIMD221063 (JEP2107+JEP2108) and pOPO330 (JEP2109+JEP2110) with pBBR1MCS-2 digested with NsiI and BamHI using NEBuilder Hifi. pOPO394 was constructed by assembling PCR products amplified from gBlock10 (JEP2111+JEP2112, JEP2113+JEP2114) and pOPO227 (JEP2115+JEP2116) with pBBR1MCS-2 digested with NsiI and BamHI using NEBuilder Hifi. pOPO435 was constructed by assembling PCR products amplified from gBlock11 (JEP2154+JEP2155, JEP2156+JEP2157) and pOPO337 (JEP2158+JEP2159) with pBBR1MCS-2 digested with NsiI and BamHI using NEBuilder Hifi. pOPO395 was constructed by assembling a PCR product amplified from gDNA of V. parahaemolyticus RIMD221063 (JEP2101+JEP2102) and pETDuet-1 digested with XbaI and AvrII using NEBuilder Hifi. pOPO397 was constructed by assembling PCR products amplified from gBlock10 (JEP2103+JEP2104, JEP2105+JEP2106) with pETDuet-1 digested with XbaI and AvrII using NEBuilder Hifi. pOPO438 was constructed by assembling PCR products amplified from gBlock11 (JEP2160+JEP2161, JEP2162+JEP2163) with pETDuet-1 digested with XbaI and AvrII using NEBuilder Hifi.

pOPO374 was constructed by ligating a PCR product amplified from pCDFDuet-1 (JEP1577+JEP1891) digested with BsaI and two pairs of phosphorylated annealed oligos (JEP1995+JEP1996, JEP1997+JEP1998). pOPO376 and pOPO378 were constructed with the same method, but with oligos (JEP2003+JEP2004, JEP2005+JEP2006) and (JEP2007+JEP2008, JEP2009+JEP2010).

pMTP281-286 were constructed by ligating a PCR product amplified from pCDFDuet-1 (JEP2032+JEP2033) digested with BsaI with four annealed oligos (JEP2063, JEP2064, JEP2065, JEP2066: pMTP281; JEP2078, JEP2079, JEP2080, JEP2081: pMTP282; JEP2035, JEP2036, JEP2037, JEP2038: pMTP283; JEP2049, JEP2050, JEP2051, JEP2052: pMTP284; JEP2067, JEP2068, JEP2069, JEP2066: pMTP285; JEP2082, JEP2083, JEP2084, JEP2081: pMTP286).

### Quantification and Statistical Analysis

Statistical details are listed in the Figure Legends. When stated, experiments were performed with three biological replicates (n=3).

Plasmids used in this disclosure are the following. Plasmid sequences are provided in the sequence listing.

Plasmid Table

| Plasmid | Description |
|---|---|
| pMS26 | Tn7 shuttle vector containing Tn7 end sequences flanking a cloning |
| | site and tnsABCD. |
| pMTP112 (pMS26 | pMS26 containing Tn6900 right (112 bp) and left (120 bp) end |
| miniTn6900(kanR)) | sequences flanking kanR cassette between Tn7 ends. |
| pMTP113 | pMS26 containing Tn6677 right (127 bp) and left (145 bp) end |
| | sequences flanking kanR cassette between Tn7 ends. |
| pKD46 | Temperature-sensitive plasmid containing RED recombination genes |
| | under arabinose inducible control. |
| pTSC29 | Temperature-sensitive plasmid with chloramphenicol resistance |
| | marker. |
| pBAD322S | Expression vector with pBR322 replicon, arabinose expression |
| | system and spectinomycin resistance marker (GenBank: DQ131584). |
| pMTP114 | pTSC29 containing two 1,000 bp fragments from F plasmid flanking |
| | a 747 bp BsaI-DHFR-BsaI cassette and 1,213 bp specR cassette. |
| pMTP115 | pMTP114 with 3,310 bp lacZ including promoter inserted over BsaI- |
| | DHFR-BsaI cassette. |
| pMTP116 | pMTP114 with 52 bp sequence upstream cysH from A. salmonicida |
| | S44 pS44-1 inserted over BsaI-DHFR-BsaI cassette. |
| pMTP117 | pMTP114 with 68 bp bp 3′ ffs sequence from A. salmonicida S44 |
| | inserted over BsaI-DHFR-BsaI cassette. |
| pMTP118 | pMTP114 with 70 bp 3′ guaC sequence from V. cholerae HE-45 |
| | inserted over BsaI-DHFR-BsaI cassette. |
| pTA106 | Expression vector with pSC101 replicon, lactose promoter, |
| | ampicillin resistance marker. |
| pMTP130 (pTA106 | pTA106 with codon-optimized tnsABC from Tn6900 (FIG. 3B, 3C, |
| TnsABC(Tn6900)) | 3D, 3E, 4B, FIG. 8, 8E). |
| pBAD322G | Expression vector with pBR322 replicon, arabinose expression |
| | system and gentamycin resistance marker (GenBank: DQ119284.1). |
| pMTP140 | pBAD322G with codon-optimized tniQ-cas8/5-cas7-cas6 operon |
| (pBAD322G TniQ- | from Tn6900 inserted (FIG. 3B, 3C, 3D, 3E, 4B, FIG. 8D, 8E). |
| Cascade(Tn6900)) | |
| pBAD33 | Expression vector with pACYC replicon, arabinose expression |
| | system and chloramphenicol resistance marker. |
| pMTP150 | pBAD33 with CRISPR array from Tn6900 with BsaI-DHFR-BsaI |
| | cassette replacing spacer one with additional BsaI downstream of |
| | MCS removed. |
| pMTP151 | ρMTP150 with first spacer from Tn6900 CRISPR array inserted, |
| | restoring wild-type CRISPR array (FIG. 3B). |
| pMTP160 | pBAD33 with CRISPR repeat one and CRISPR repeat two (typical |
| | configuration) from Tn6900 flanking BsaI-DHFR-BsaI cassette with |
| | additional BsaI downstream of MCS removed. |
| pMTP161 | pMTP160 with first spacer from Tn6900 CRISPR array targeting |
| | plasmid cysH (FIG. 3C). |
| pMTP162 | pMTP160 with second spacer from Tn6900 CRISPR array targeting |
| | chromosomal ffs (ffs^{WT}) (FIG. 3C, 3D). |
| pMTP163 | pMTP160 with second spacer from Tn6900 CRISPR array targeting |
| | chromosomal ffs, with mismatches to protospacer corrected (ffs^{Exact}) |
| | (FIG. 3C, 3D). |
| pMTP164 | pMTP160 with lacZ-targeting spacer #3 (with TACC PAM) (FIG. |
| | 3D). |
| pMTP165 | pMTP160 with lacZ-targeting spacer #4 (with TACC PAM) (FIG. |
| | 3D, 4B). |
| pMTP170 | pBAD33 with CRISPR repeat two and CRISPR repeat three |
| (pBAD33 | (atypical configuration) from Tn6900 flanking BsaI-DHFR-BsaI |
| CRISPR(Tn6900)- | cassette with additional BsaI downstream of MCS removed. |
| entry) | |
| pMTP171 | pMTP170 with first spacer from Tn6900 CRISPR array targeting |
| | plasmid cysH (FIG. 3C). |
| pMTP172 | pMTP170 with second spacer from Tn6900 CRISPR array targeting |
| | chromosomal ffs (ffs^{WT}) (FIG. 3C, 3D). |
| pMTP173 | pMTP170 with second spacer from Tn6900 CRISPR array targeting |
| | chromosomal ffs, with mismatches to protospacer corrected (ffs^{Exact}) |
| | (FIG. 3C, 3D). |
| pMTP174 | pMTP170 with lacZ-targeting spacer #1 (with TACC PAM) (FIG. |
| | 3E, FIG. 8D). |
| pMTP175 | pMTP170 with lacZ-targeting spacer #2 (with TACC PAM) (FIG. |
| | 3E, FIG. 8D). |
| pMTP176 | pMTP170 with lacZ-targeting spacer #3 (with TACC PAM) (FIG. |
| | 3D, 3E). |
| pMTP177 | pMTP170 with lacZ-targeting spacer #4 (with TACC PAM) (FIG. |
| | 3D, 3E, 4B, FIG. 8D). |
| pMTP178 | pMTP170 with lacZ-targeting spacer #5 (with TACC PAM) (FIG. |
| | 3E). |
| pMTP179 | pMTP170 with lacZ-targeting spacer #6 (with TACC PAM) (FIG. |
| | 3E). |
| pMTP180 | pMTP170 with galK top strand-targeting spacer (with TACC PAM) |
| | (FIG. 8E). |
| pMTP181 | pMTP170 with galK bottom strand-targeting spacer (with TACC |
| | PAM) (FIG. 8E). |
| pMTP182 | pMTP170 with srlD top strand-targeting spacer (with TTCC PAM) |
| | FIG. 8E). |
| pMTP183 | pMTP170 with srlD bottom strand-targeting spacer (with TACC |
| | PAM) (FIG. 8E). |
| pMTP190 | pBAD33 with additional BsaI site outside of MCS removed. |
| pMTP191 | pMTP190 with lacZ-targeting spacer #4 flanked by Tn6900 typical |
| | repeats with changes at 3 nucleotide positions to corresponding |
| | atypical base (typical*) (FIG. 4B). |
| pMTP192 | pMTP190 with lacZ-targeting spacer #4 flanked by Tn6900 atypical |
| | repeats with changes at 3 nucleotide positions to corresponding |
| | typical base (atypical*) (FIG. 4B). |
| pSL0527 (pDonor) | pUC19 with Tn6677 right end left ends flanking chloramphenicol |
| | resistance cassette. |
| pSL0283 | pCOLADuet-1 with Tn6677 tnsABC. |
| (pTnsABC) | |
| pSL0284 | pCDFDuet-1 with Tn6677 tniQ-cas8/5-cas7-cas6 and Tn6677 |
| (pQCascade_entry) | CRISPR repeat entry module. |
| pBAD322 | Expression vector with pBR322 replicon, arabinose expression |
| | system and ampicillin resistance marker (GenBank: DQ119282). |
| pMTP230 | pBAD33 with araC and lac promoter removed with lac promoter and |
| | Tn6677 tnsABC inserted (FIG. 4C, 4D). |
| pMTP240 | pBAD322 with Tn6677 tniQ-cas8/5-cas7-cas6 inserted (FIG. 4C, |
| | 4D). |
| pBBR1MCS-3 | Expression vector with pBBR replicon, lactose expression system |
| | and tetracycline resistance marker. |
| pCDFDuet-1 | Expression vector with pCDF replicon, tandem T7 promoters, and |
| | spectinomycin resistance marker. |
| pMTP250 | Expression vector with pCDF replicon, arabinose expression system |
| | and tetracycline resistance marker. |
| pMTP260 | pMTP250 with two copies of Tn6677 repeat one (typical |
| | configuration) flanking BsaI sites. |
| pMTP261 | pMTP260 with fourth spacer from Tn6677 CRISPR array targeting |
| | chromosomal guaC (guaC^{WT}) (FIG. 4C). |
| pMTP262 | pMTP260 with fourth spacer from Tn6677 CRISPR array targeting |
| | chromosomal guaC, with mismatches to protospacer corrected |
| | (guaC^{Exact}) (FIG. 4C). |
| pMTP263 | pMTP260 with lacZ-targeting spacer #3 (with TACC PAM) (FIG. |
| | 4D). |
| pMTP264 | pMTP260 with lacZ-targeting spacer #4 (with TACC PAM) (FIG. |
| | 4D). |
| pMTP270 | pMTP250 with Tn6677 repeat five and repeat six (atypical |
| | configuration) flanking BsaI sites. |
| pMTP271 | pMTP270 with fourth spacer from Tn6677 CRISPR array targeting |
| | chromosomal guaC (guaC^{WT}) (FIG. 4C, 4D). |
| pMTP272 | pMTP270 with fourth spacer from Tn6677 CRISPR array targeting |
| | chromosomal guaC, with mismatches to protospacer corrected |
| | (guaC^{Exact}) (FIG. 4C). |
| pMTP273 | pMTP270 with lacZ-targeting spacer #3 (with TACC PAM) (FIG. |
| | 4D). |
| pMTP274 | pMTP270 with lacZ-targeting spacer #4 (with TACC PAM) (FIG. |
| | 4D). |
| pMTP275 | pMTP250 with lacZ-targeting spacer #4 flanked by Tn6677 typical |
| | repeats with changes at 3 nucleotide positions to corresponding |
| | atypical base (typical*) (FIG. 4D). |
| pMTP276 | pMTP250 with lacZ-targeting spacer #4 flanked by Tn6677 atypical |
| | repeats with changes at 3 nucleotide positions to corresponding |
| | typical base (atypical*) (FIG. 4D). |
| FΔ(finO- | F plasmid (GenBank: AP001918.1) with a 44,962 bp section from |
| fxsA)::lacZ specR | finO to fxsA replaced with 3,310 bp lacZ including promoter |
| | (GenBank: U00096.3) and 1,213 bp specR cassette (GenBank: |
| | DQ131584.1) (FIG. 3B, 3D, 3E, 4B, 4D). |
| FΔ(finO- | F plasmid (GenBank: AP001918.1) with a 44,962 bp section from |
| fxsA)::cysH^{As} specR | finO to fxsA replaced with 52 bp upstream cysH sequence from |
| | A. salmonicida S44 pS44-1 including protospacer (GenBank: |
| | CP022176.1) and 1,213 bp specR cassette (GenBank: DQ131584.1) |
| | (FIG. 3B, 3C). |
| FΔ(finO-fxsA)::ffs^{As} | F plasmid (GenBank: AP001918.1) with a 44,962 bp section from |
| specR | finO to fxsA replaced with 68 bp 3′ ffs sequence from A. salmonicida |
| | S44 including protospacer (GenBank: CP022181.1) and 1,213 bp |
| | specR cassette (GenBank: DQ131584.1) (FIG. 3B, 3C, 3D). |
| FΔ(finO- | F plasmid (GenBank: AP001918.1) with a 44,962 bp section from |
| fxsA)::guaC^{Vc} | finO to fxsA replaced with 70 bp 3′ guaC sequence from V. cholerae |
| specR | HE-45 including protospacer (GenBank: ALED01000027.1) and |
| | 1,213 bp specR cassette (GenBank: DQ131584.1) (FIG. 4C). |
| pCsy_complex | Carries csy1, csy2, csy3, and csy4 from Pseudomonas aeruginosa |
| | PA14 under T7 promoter (FIG. 5). |
| pCas1_Cas2/3 | Carries cas1, cas2/3 from Pseudomonas aeruginosa PA14. cas1 has |
| | Strep-Tag II. |
| pCOLADuet-1 | Expression vector with pCOLA replicon, tandem T7 promoters, and |
| | kanamycin resistance marker. |
| pACYCDuet-1 | Expression vector with pACYC replicon, tandem T7 promoters, and |
| | chloramphenicol resistance marker. |
| pOPO390 | pCOLADuet-1 with 3′ guaC from V. cholerae HE-45 protospacer |
| | sequence with CC PAM (FIG. 5). |
| pOPO275 | pCOLADuet-1 with 3′ ffs from A. salmonicida S44 protospacer |
| | sequence with CC PAM (FIG. 5). |
| pOPO322 | pACYCDuet-1 with cas1 and cas2/3 from P. aeruginosa PA14 |
| | (FIG. 5). |
| pOPO374 | pCDFDuet-1 with two copies of CRISPR repeat one from |
| | P. aeruginosa PA14 flanking a spacer matching chromosomal ffs |
| | perfectly (ffs^{Exact}) (FIG. 5). |
| pOPO376 | pCDFDuet-1 with CRISPR repeat one and CRISPR repeat two |
| | (typical configuration) from Tn6900 flanking second spacer from |
| | Tn6900 CRISPR array targeting chromosomal ffs, with mismatches |
| | to protospacer corrected (ffs^{Exact}) (FIG. 5). |
| pOPO378 | pCDFDuet-1 with CRISPR repeat two and CRISPR repeat three |
| | (atypical configuration) from Tn6900 flanking second spacer from |
| | Tn6900 CRISPR array targeting chromosomal ffs, with mismatches |
| | to protospacer corrected (ffs^{Exact}) (FIG. 5). |
| pMTP281 | pCDFDuet-1 with two copies of CRISPR repeat one (typical |
| | configuration) from Tn6677 flanking fourth spacer from Tn6677 |
| | CRISPR array targeting chromosomal guaC, with mismatches to |
| | protospacer corrected (guaC^{Exact}) (FIG. 5). |
| pMTP282 | pCDFDuet-1 with CRISPR repeat five and CRISPR repeat six |
| | (atypical configuration) from Tn6677 flanking fourth spacer from |
| | Tn6677 CRISPR array targeting chromosomal guaC, with |
| | mismatches to protospacer corrected (guaC^{Exact}) (FIG. 5). |
| pMTP283 | pCDFDuet-1 with CRISPR repeat one and CRISPR repeat two |
| | (typical configuration) from Tn6900 flanking second spacer from |
| | Tn6900 CRISPR array targeting chromosomal ffs (ffs^{WT}) (FIG. 5). |
| pMTP284 | pCDFDuet-1 with CRISPR repeat two and CRISPR repeat three |
| | (atypical configuration) from Tn6900 flanking second spacer from |
| | Tn6900 CRISPR array targeting chromosomal ffs (ffs^{WT}) (FIG. 5). |
| pMTP285 | pCDFDuet-1 with two copies of CRISPR repeat one (typical |
| | configuration) from Tn6677 flanking fourth spacer from Tn6677 |
| | CRISPR array targeting chromosomal guaC (guaC^{WT}) (FIG. 5). |
| pMTP286 | pCDFDuet-1 with CRISPR repeat five and CRISPR repeat six |
| | (atypical configuration) from Tn6677 flanking fourth spacer from |
| | Tn6677 CRISPR array targeting chromosomal guaC (guaC^{WT}) |
| | (FIG. 5). |
| pOPO256 | pBAD33 with modified weaker RBS (iGEM BBa_B0033) and xre |
| | gene from Tn6900 inserted (FIG. 6F). |
| pOPO258 | pBAD33 with modified weaker RBS (iGEM BBa_B0033) and xre |
| | gene from Vibrio sp. 10N.286.45.B6 inserted (FIG. 6F). |
| pOPO364 | pBAD33 with modified weaker RBS (iGEM BBa_B0033) and xre |
| | gene from V. parahaemolyticus RIMD221063 inserted (FIG. 6E). |
| pOPO345 | pBAD33 with modified weaker RBS (iGEM BBa_B0033) and xre |
| | gene from Tn6677 inserted (FIG. 6E). |
| pBAD24 | Expression vector with pBR322 replicon lacking rop gene copy |
| | control, arabinose expression system and ampicillin resistance |
| | marker (GenBank: DQ119282). |
| pOPO221 | pBAD24 with a promoterless, start codon-lacking lacZ inserted. |
| pOPO228 | pOPO221 with xre promoter from Vibrio sp. 10N.286.45.B6 fused to |
| | lacZ (FIG. 6F). |
| pOPO227 | pOPO221 with tniQ promoter from Vibrio sp. 10N.286.45.B6 fused |
| | to lacZ (FIG. 6F). |
| pOPO229 | pOPO221 with xre promoter from Tn6900 fused to lacZ (FIG. 6F). |
| pOPO230 | pOPO221 with tniQ promoter from Tn6900 fused to lacZ (FIG. |
| | 6F). |
| pOPO332 | pOPO221 with xre promoter from Tn6900 with proximal putative |
| | Xre binding site mutated fused to lacZ (FIG. 6F). |
| pOPO334 | pOPO221 with tniQ promoter from Tn6900 with putative Xre |
| | binding site mutated fused to lacZ (FIG. 6F). |
| pOPO329 | pOPO221 with xre promoter from V. parahaemolyticus |
| | RIMD221063 fused to lacZ (FIG. 6E). |
| pOPO330 | pOPO221 with atypical guide RNA promoter from |
| | V. parahaemolyticus RIMD221063 fused to lacZ (FIG. 6E). |
| pOPO341 | pOPO221 with xre promoter from Tn6677 fused to lacZ (FIG. 6E). |
| pOPO337 | pOPO221 with atypical guide RNA promoter from Tn6677 fused to |
| | lacZ (FIG. 6E). |
| pET22b(+) | Expression vector with pBR322 replicon, T7 expression system, and |
| | ampicillin resistance marker. |
| pOPO223 | pET22b(+) with C-terminal His6-tagged (SEQ ID NO: 5806) codon- |
| | optimized xre from Tn6900 (FIG. 6D). |
| pOPO239 | pET22b(+) with C-terminal His6-tagged (SEQ ID NO: 5806) xre |
| | from Vibrio sp. 10N.286.45.B6 (FIG. 6D). |
| pOPO331 | pET22b(+) with C-terminal His6-tagged (SEQ ID NO: 5806) xre |
| | from V. parahaemolyticus RIMD221063 (FIG. 6C). |
| pOPO360 | pET22b(+) with C-terminal His6-tagged (SEQ ID NO: 5806) xre |
| | from Tn6677 (FIG. 6C). |
| pETDuet-1 | Expression vector with ColE1 replicon, tandem T7 promoters, and |
| | ampicillin resistance marker. |
| pBBR1MCS-2 | Expression vector with pBBR replicon, lactose expression system |
| | and kanamycin resistance marker. |
| pOPO392 | pBBR1MCS-2 with xre from V. parahaemolyticus RIMD221063 and |
| | pAttGuide(VP) fused to lacZ (FIG. 7). |
| pOPO394 | pBBR1MCS-2 with xre from Vibrio sp. 10N.286.45.B6 and |
| | pTniQ(VB6) fused to lacZ (FIG. 7). |
| pOPO435 | pBBR1MCS-2 with xre from V. cholerae Tn6677 and |
| | pAttGuide(Vc) fused to lacZ (FIG. 7). |
| pOPO395 | pETDuet-1 with xre from V. parahaemolyticus RIMD221063 under |
| | native promoter control (FIG. 7). |
| pOPO397 | pETDuet-1 with xre from Vibrio sp. 10N.286.45.B6 under native |
| | promoter control (FIG. 7). |
| pOPO438 | pETDuet-1 with xre from V. cholerae HE-45 Tn6677 under native |
| | promoter control (FIG. 7). |

The following oligonucleotides were used in this disclosure.

Oligonucleotide Table.

| | |
|---|---|
| JEP2033 | TGGTCTCACCATAGGCTGCTGCCACC (SEQ |
| | ID NO: 5684) |
| JEP2035 | ccgggtgaactgccgtataggcagccaagaa |
| | aAGG (SEQ ID NO: 5685) |
| JEP2036 | ccaacttggatgatttcttccagtccttttc |
| | ttggctgcctatacggcagttcac (SEQ |
| | ID NO: 5686) |
| JEP2037 | actggaagaaatcatccaagttggggactgt |
| | gaactgccgtataggcagccaagatt (SEQ |
| | ID NO: 5687) |
| JEP2038 | atggaatcttggctgcctatacggcagttca |
| | cagtcc (SEQ ID NO: 5688) |
| JEP2049 | ccgggtgaactgccgtataggcagccaagat |
| | tagg (SEQ ID NO: 5689) |
| JEP2050 | ccaacttggatgatttcttccagtcctaatc |
| | ttggctgcctatacggcagttcac (SEQ |
| | ID NO: 5690) |
| JEP2051 | actggaagaaatcatccaagttggggactat |
| | tttctgccgtaaaggcagatattatt (SEQ |
| | ID NO: 5691) |
| JEP2052 | atggaataatatctgcctttacggcagaaaa |
| | tagtcc (SEQ ID NO: 5692) |
| JEP2063 | ccggGTGAACTGCCGAGTAGGTAGCTGATAA |
| | CAAG (SEQ ID NO: 5693) |
| JEP2064 | TTGAACTCGGATAAACGTTGTACGCTTGTTA |
| | TCAGCTACCTACTCGGCAGTTCAC (SEQ |
| | ID NO: 5694) |
| JEP2065 | CGTACAACGTTTATCCGAGTTCAAGAGCAGT |
| | GAACTGCCGAGTAGGTAGCTGATAAC (SEQ |
| | ID NO: 5695) |
| JEP2066 | atggGTTATCAGCTACCTACTCGGCAGTTCA |
| | CTGCTC (SEQ ID NO: 5696) |
| JEP2067 | ccggGTGAACTGCCGAGTAGGTAGCTGATAA |
| | CAAA (SEQ ID NO: 5697) |
| JEP2068 | TTGTACTCGAATGAAGGTGGTTCGTTTGTTA |
| | TCAGCTACCTACTCGGCAGTTCAC (SEQ |
| | ID NO: 5698) |
| JEP2069 | CGAACCACCTTCATTCGAGTACAAGAGCAGT |
| | GAACTGCCGAGTAGGTAGCTGATAAC (SEQ |
| | ID NO: 5699) |
| JEP2078 | ccggTCATTACTACTGCAAAGTAGCTGATAA |
| | CAAG (SEQ ID NO: 5700) |
| JEP2079 | TTGAACTCGGATAAACGTTGTACGCTTGTTA |
| | TCAGCTACTTTGCAGTAGTAATGA (SEQ |
| | ID NO: 5701) |
| JEP2080 | CGTACAACGTTTATCCGAGTTCAAGAGCACT |
| | TTACTGCTGAATAAGTAGATAACTAC (SEQ |
| | ID NO: 5702) |
| JEP2081 | atggGTAGTTATCTACTTATTCAGCAGTAAA |
| | GTGCTC (SEQ ID NO: 5703) |
| JEP2082 | ccggTCATTACTACTGCAAAGTAGCTGATAA |
| | CAAA (SEQ ID NO: 5704) |
| JEP2083 | TTGTACTCGAATGAAGGTGGTTCGTTTGTTA |
| | TCAGCTACTTTGCAGTAGTAATGA (SEQ |
| | ID NO: 5705) |
| JEP2084 | CGAACCACCTTCATTCGAGTACAAGAGCACT |
| | TTACTGCTGAATAAGTAGATAACTAC (SEQ |
| | ID NO: 5706) |
| JEP2101 | GAGCGGATAACAATTCCCCTTCAACTAGAGT |
| | AAATGAACTC (SEQ ID NO: 5707) |
| JEP2102 | TGCTCAGCGGTGGCAGCAGCTTAGCAACTCT |
| | CGACAGTAG (SEQ ID NO: 5708) |
| JEP2103 | GAGCGGATAACAATTCCCCTTTAACTTCTCA |
| | GCTTTTCCAAC (SEQ ID NO: 5709) |
| JEP2104 | CCCAGCGAGGCTCAAAGCTGCGCGTAAAAAA |
| | G (SEQ ID NO: 5710) |
| JEP2105 | CAGCTTTGAGCCTCGCTGGGATGGGGTT |
| | (SEQ ID NO: 5711) |
| JEP2106 | TGCTCAGCGGTGGCAGCAGCTGACTCTTACA |
| | GGCAATAAAAACTTAGGATTG (SEQ ID |
| | NO: 5712) |
| JEP2107 | CGGCCGCTCTAGAACTAGTGTTAGCAACTCT |
| | CGACAGTAG (SEQ ID NO: 5713) |
| JEP2108 | TCAAAAATAATCAACTAGAGTAAATGAACTC |
| | (SEQ ID NO: 5714) |
| JEP2109 | CTCTAGTTGATTATTTTTGACACCAGACC |
| | (SEQ ID NO: 5715) |
| JEP2110 | ATTACAACAGTTTTTATGCAAGTTTAAAAAA |
| | TCAATACTATTACCATC (SEQ ID NO: |
| | 5716) |
| JEP2111 | CGGCCGCTCTAGAACTAGTGTGACTCTTACA |
| | GGCAATAAAAACTTAGGATTG (SEQ ID |
| | NO: 5717) |
| JEP2112 | CAGCTTTGAGCCTCGCTGGGATGGGGTT |
| | (SEQ ID NO: 5711) |
| JEP2113 | CCCAGCGAGGCTCAAAGCTGCGCGTAAAAAA |
| | G (SEQ ID NO: 5710) |
| JEP2114 | TTAATAAACTAGAGTTTGTAGAAACGCAAAA |
| | AG (SEQ ID NO: 5718) |
| JEP2115 | TACAAACTCTAGTTTATTAAAGTTCACAATT |
| | TGG (SEQ ID NO: 5719) |
| JEP2116 | ATTACAACAGTTTTTATGCATTATTTTTGAC |
| | ACCAGACC (SEQ ID NO: 5720) |
| JEP2119 | CTGAACCAAGCGTACAACGTTTATCCGAGTT |
| | CAAGAGCA (SEQ ID NO: 5721) |
| JEP2120 | GGCTGCTCTTGAACTCGGATAAACGTTGTAC |
| | GCTTGGTT (SEQ ID NO: 5722) |
| JEP2154 | CGGCCGCTCTAGAACTAGTGTTAGATATCCA |
| | TTGGTCACATTC (SEQ ID NO: 5723) |
| JEP2155 | CTAAGTCTTTCATTTAAATCGAATCACAAAA |
| | TG (SEQ ID NO: 5724) |
| JEP2156 | GATTTAAATGAAAGACTTAGCGAAACGG |
| | (SEQ ID NO: 5725) |
| JEP2157 | CTTTACTGCTGAATAAGTAGATAACTACGGA |
| | AGAAGCTCTCTAAC (SEQ ID NO: |
| | 5726) |
| JEP2158 | GTTATCTACTTATTCAGCAGTAAAGTTATTT |
| | TTGACACCAGACC (SEQ ID NO: 5727) |
| JEP2159 | ATTACAACAGTTTTTATGCACACACATCAAC |
| | ACCGTTAC (SEQ ID NO: 5728) |
| JEP2160 | GAGCGGATAACAATTCCCCTTTACGGAAGAA |
| | GCTCTCTAAC (SEQ ID NO: 5729) |
| JEP2161 | GATTTAAATGAAAGACTTAGCGAAACGG |
| | (SEQ ID NO: 5725) |
| JEP2162 | CTAAGTCTTTCATTTAAATCGAATCACAAAA |
| | TG (SEQ ID NO: 5724) |
| JEP2163 | TGCTCAGCGGTGGCAGCAGCTTAGATATCCA |
| | TTGGTCACATTC (SEQ ID NO: 5730) |

Synthesized gene fragments (gBlocks) used in this disclosure are referred to by the following names. Sequences are provides in the sequence listing.

| Gene fragments | Description |
|---|---|
| gBlock1 | miniTn6900(kanR) |
| gBlock2 | tnsABC(Tn6900) fragment one |
| gBlock3 | tnsABC(Tn6900) fragment two |
| gBlock4 | tniQ(Tn6900) |
| gBlock5 | Cascade(Tn6900) fragment one |
| gBlock6 | Cascade(Tn6900) fragment two |
| gBlock7 | CRISPR array Tn6900 fragment one |
| gBlock8 | CRISPR array Tn6900 fragment two |
| gBlock9 | xre(As) |
| gBlock10 | xre(VB6), pXre(VB6), pTniQ(VB6), |
| | pXre(As), pTniQ(As) |
| gBlock11 | xre(Vc), pXre(Vc), pAttguide(Vc), |
| | pXre*(As), pTniQ*(As), |

The following list of cited references is not an indication that any of the references are material to patentability.
Bainton, R., Gamas, P., and Craig, N. L. (1991). Tn7 transposition in vitro proceeds through an excised transposon intermediate generated by staggered breaks in DNA. Cell 65, 805-816.
Bainton, R. J., Kubo, K. M., Feng, J.-N., and Craig, N. L. (1993). Tn7 transposition: target DNA recognition is mediated by multiple Tn7-encoded proteins in a purified in vitro system. Cell 72, 931-943.
Boehmer, T., Vogler, A. J., Thomas, A., Sauer, S., Hergenroether, M., Straubinger, R. K., Birdsell, D., Keim, P., Sahl, J. W., Williamson, C. H. D., et al. (2018). Phenotypic characterization and whole genome analysis of extended-spectrum beta-lactamase-producing bacteria isolated from dogs in Germany. PLoS One 13, e0206252.
Borges, A. L., Davidson, A. R., and Bondy-Denomy, J. (2017). The Discovery, Mechanisms, and Evolutionary Impact of Anti-CRISPRs. Annu Rev Virol 4, 37-59.
Chowdhury, S., Carter, J., Rollins, M. F., Golden, S. M., Jackson, R. N., Hoffmann, C., Nosaka, L., Bondy-Denomy, J., Maxwell, K. L., Davidson, A. R., et al. (2017). Structure Reveals Mechanisms of Viral Suppressors that Intercept a CRISPR RNA-Guided Surveillance Complex. Cell 169, 47-57.e11.
Datsenko, K. A., and Wanner, B. L. (2000). One-step inactivation of chromosomal genes in Escherichia coli K-12 using PCR products. Proc Natl Acad Sci USA 97, 6640-6645.
Engler, C., Kandzia, R., and Marillonnet, S. (2008). A one pot, one step, precision cloning method with high throughput capability. PloS one 3, e3647-e3647.
Faure, G., Makarova, K. S., and Koonin, E. V. (2019a). CRISPR-Cas: Complex Functional Networks and Multiple Roles beyond Adaptive Immunity. J Mol Biol 431, 3-20.
Faure, G., Shmakov, S. A., Yan, W. X., Cheng, D. R., Scott, D. A., Peters, J. E., Makarova, K. S., and Koonin, E. V. (2019b). CRISPR-Cas in mobile genetic elements: counter-defence and beyond. Nat Rev Microbiol 17, 513-525.
Fineran, P. C., Gerritzen, M. J., Suarez-Diez, M., Kunne, T., Boekhorst, J., van Hijum, S. A., Staals, R. H., and Brouns, S. J. (2014). Degenerate target sites mediate rapid primed CRISPR adaptation. Proc Natl Acad Sci USA 111, E1629-1638.
Gleditzsch, D., Muller-Esparza, H., Pausch, P., Sharma, K., Dwarakanath, S., Urlaub, H., Bange, G., and Randau, L. (2016). Modulating the Cascade architecture of a minimal Type I-F CRISPR-Cas system. Nucleic Acids Res 44, 5872-5882.
Hoyland-Kroghsbo, N. M., Paczkowski, J., Mukherjee, S., Broniewski, J., Westra, E., Bondy-Denomy, J., and Bassler, B. L. (2017). Quorum sensing controls the Pseudomonas aeruginosa CRISPR-Cas adaptive immune system. Proc Natl Acad Sci USA 114, 131-135.
Jackson, R. N., Golden, S. M., van Erp, P. B., Carter, J., Westra, E. R., Brouns, S. J., van der Oost, J., Terwilliger, T. C., Read, R. J., and Wiedenheft, B. (2014). Structural biology. Crystal structure of the CRISPR RNA-guided surveillance complex from Escherichia coli. Science 345, 1473-1479.
Klompe, S. E., Vo, P. L. H., Halpin-Healy, T. S., and Sternberg, S. H. (2019). Transposon-encoded CRISPR-Cas systems direct RNA-guided DNA integration. Nature 571, 219-225.
Kuznedelov, K., Mekler, V., Lemak, S., Tokmina-Lukaszewska, M., Datsenko, K. A., Jain, I., Savitskaya, E., Mallon, J., Shmakov, S., Bothner, B., et al. (2016). Altered stoichiometry Escherichia coli Cascade complexes with shortened CRISPR RNA spacers are capable of interference and primed adaptation. Nucleic Acids Res 44, 10849-10861.
Makarova, K. S., Wolf, Y. I., Iranzo, J., Shmakov, S. A., Alkhnbashi, O. S., Brouns, S. J. J., Charpentier, E., Cheng, D., Haft, D. H., Horvath, P., et al. (2020). Evolutionary classification of CRISPR-Cas systems: a burst of class 2 and derived variants. Nat Rev Microbiol 18, 67-83.
Malke, H. (1993). Jeffrey H. Miller, A Short Course in Bacterial Genetics—A Laboratory Manual and Handbook for Escherichia coli and Related Bacteria. Cold Spring Harbor 1992. Cold Spring Harbor Laboratory Press. ISBN: 0-87969-349-5. Journal of Basic Microbiology 33, 278-278.
Martynov, A., Severinov, K., and Ispolatov, I. (2017). Optimal number of spacers in CRISPR arrays. PLoS Comput Biol 13, e1005891.
McGeehan, J. E., Streeter, S. D., Thresh, S. J., Taylor, J. E., Shevtsov, M. B., and Kneale, G. G. (2011). Structural analysis of a novel class of R-M controller proteins: C.Csp231I from Citrobacter sp. RFL231. J Mol Biol 409, 177-188.
Mitra, R., McKenzie, G. J., Yi, L., Lee, C. A., and Craig, N. L. (2010). Characterization of the TnsD-attTn7 complex that promotes site-specific insertion of Tn7. Mobile DNA 1, 18.
Mulepati, S., Heroux, A., and Bailey, S. (2014). Structural biology. Crystal structure of a CRISPR RNA-guided surveillance complex bound to a ssDNA target. Science 345, 1479-1484.
Parks, A. R., Li, Z., Shi, Q., Owens, R. M., Jin, M. M., and Peters, J. E. (2009). Transposition into replicating DNA occurs through interaction with the processivity factor. Cell 138, 685-695.
Patterson, A. G., Jackson, S. A., Taylor, C., Evans, G. B., Salmond, G. P. C., Przybilski, R., Staals, R. H. J., and Fineran, P. C. (2016). Quorum Sensing Controls Adaptive Immunity through the Regulation of Multiple CRISPR-Cas Systems. Mol Cell 64, 1102-1108.
Peters, J. E. (2014). Tn7. Microbiology Spectrum 2, 1-20.
Peters, J. E. (2019). Targeted transposition with Tn7 elements: safe sites, mobile plasmids, CRISPR/Cas and beyond. Mol Microbiol 112, 1635-1644.
Peters, J. E., and Craig, N. L. (2001). Tn7 recognizes target structures associated with DNA replication using the DNA binding protein TnsE. Genes & Dev 15, 737-747.
Peters, J. E., Makarova, K. S., Shmakov, S., and Koonin, E. V. (2017). Recruitment of CRISPR-Cas systems by Tn7-like transposons. Proceedings of the National Academy of Sciences 114, E7358.
Rice, P. A., Craig, N. L., and Dyda, F. (2020). Comment on “RNA-guided DNA insertion with CRISPR-associated transposases”. Science 368.
Rodic, A., Blagojevic, B., Zdobnov, E., Djordjevic, M., and Djordjevic, M. (2017). Understanding key features of bacterial restriction-modification systems through quantitative modeling. BMC Syst Biol 11, 377.
Shi, Q., Straus, M. R., Caron, J. J., Wang, H., Chung, Y. S., Guarne, A., and Peters, J. E. (2015). Conformational toggling controls target site choice for the heteromeric transposase element Tn7. Nucleic Acids Res.
Stellwagen, A. E., and Craig, N. L. (1998). Mobile DNA elements: controlling transposition with ATP-dependent molecular switches. Trends Biochem Sci 23, 486-490.
Strecker, J., Ladha, A., Gardner, Z., Schmid-Burgk, J. L., Makarova, K. S., Koonin, E. V., and Zhang, F. (2019). RNA-guided DNA insertion with CRISPR-associated transposases. Science 365, 48-53.
Strecker, J., Ladha, A., Makarova, K. S., Koonin, E. V., and Zhang, F. (2020). Response to Comment on “RNA-guided DNA insertion with CRISPR-associated transposases”. Science 368.
Streeter, S. D., Papapanagiotou, I., McGeehan, J. E., and Kneale, G. G. (2004). DNA footprinting and biophysical characterization of the controller protein C.AhdI suggests the basis of a genetic switch. Nucleic Acids Res 32, 6445-6453.
Vorontsova, D., Datsenko, K. A., Medvedeva, S., Bondy-Denomy, J., Savitskaya, E. E., Pougach, K., Logacheva, M., Wiedenheft, B., Davidson, A. R., Severinov, K., et al. (2015). Foreign DNA acquisition by the I-F CRISPR-Cas system requires all components of the interference machinery. Nucleic Acids Res 43, 10848-10860.
Waddell, C. S., and Craig, N. L. (1988). Tn7 transposition: two transposition pathways directed by five Tn7-encoded genes. Genes Dev 2, 137-149.
Westra, E. R., Pul, U., Heidrich, N., Jore, M. M., Lundgren, M., Stratmann, T., Wurm, R., Raine, A., Mescher, M., Van Heereveld, L., et al. (2010). H—NS-mediated repression of CRISPR-based immunity in Escherichia coli K12 can be relieved by the transcription activator LeuO. Mol Microbiol 77, 1380-1393.
Wiegand, T., Karambelkar, S., Bondy-Denomy, J., and Wiedenheft, B. (2020). Structures and Strategies of Anti-CRISPR-Mediated Immune Suppression. Annu Rev Microbiol.
Xiao, Y., Ng, S., Nam, K. H., and Ke, A. (2017). How type II CRISPR-Cas establish immunity through Cas1-Cas2-mediated spacer integration. Nature 550, 137-141.
Zhao, H., Sheng, G., Wang, J., Wang, M., Bunkoczi, G., Gong, W., Wei, Z., and Wang, Y. (2014). Crystal structure of the RNA-guided immune surveillance Cascade complex in Escherichia coli. Nature 515, 147-150.

While the disclosure has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A recombinant RNA polynucleotide comprising contiguously in a 5′ to 3′ direction:
i) a 5′ end segment comprising a first CRISPR repeat sequence;
ii) a spacer sequence that comprises a targeting sequence that is complementary to a protospacer in a DNA target sequence; and
iii) a 3′ end segment comprising a second CRISPR repeat sequence;
wherein the 5′ end segment or the 3′ end segment comprises one or more nucleotide changes relative to a first reference repeat sequence, and wherein when contacted with type I—F3 CRISPR-Cas proteins, the recombinant RNA polynucleotide interacts with the type I—F3 CRISPR-Cas proteins to form a functional type I—F3 CRISPR-Cas complex that effects a modification in the DNA target sequence.

2. The recombinant RNA polynucleotide of claim 1 wherein the 3′ end segment or the 5′ end segment comprises one or more nucleotide changes relative to a second reference repeat sequence.

3. The recombinant RNA polynucleotide of claim 1, wherein the 5′ end segment and the 3′ end segment each comprises one or more nucleotide changes relative to the first and second reference repeat sequences, respectively.

4. The recombinant RNA polynucleotide of claim 1, wherein the type I—F3 CRISPR-Cas proteins comprise type I-F3b CRISPR-Cas proteins to form a functional type I-F3b CRISPR-Cas complex, and wherein the CRISPR repeat sequence optionally comprises three contiguous nucleotides at a 5′ end that are not GUG.

5. The recombinant RNA polynucleotide of claim 1, wherein the engineered guide polynucleotide exhibits more efficient modification of the DNA target sequence when contacted with the DNA target sequence along with a Type IF-3b CRISPR protein complex as compared to a control guide RNA that does not comprise the one or more nucleotide changes.

6. The recombinant RNA polynucleotide of claim 5, wherein the modification comprises insertion of a DNA cargo into the DNA target sequence.

7. The recombinant RNA polynucleotide of claim 5, wherein the 5′ end segment comprises or consists of 8 nucleotides, and/or wherein the 3′ end segment comprises or consists of 20 nucleotides, and wherein the 3′ end of the 20 nucleotides is a G.

8. The RNA polynucleotide of claim 5, wherein the 3′ end segment forms a stem loop, the stem loop comprising palindromic sequences.

9. The recombinant RNA polynucleotide claim 8, wherein the first reference repeat sequence is encoded by a first occurring repeat sequence that is 3′ to a Cas6 coding sequence in an endogenous prokaryotic CRISPR array, and/or wherein the second reference repeat sequence is encoded by a second occurring repeat sequence that is 3′ to the Cas6 coding sequence in the endogenous prokaryotic CRISPR array, and wherein the endogenous prokaryotic CRISPR array is optionally a gammaproteobacteria CRISPR array.

10. The recombinant RNA polynucleotide of claim 9, wherein the gammaproteobacteria CRISPR array comprises an A. salmonicida CRISPR array.

11. The recombinant RNA polynucleotide of any one of claims 1-10, wherein the RNA polynucleotide is present in a ribonucleoprotein complex.

12. The recombinant RNA polynucleotide of claim 11, wherein proteins in the ribonucleoprotein are selected from Cas5, Cas6, Cas7, Cas8, and combinations thereof.

13. The recombinant RNA polynucleotide of claim 12, wherein the ribonucleoprotein comprises the Cas6 and wherein a stem loop comprising at least a portion of the 3′ end segment is recognized by the Cas6 in the ribonucleoprotein complex.

14. The recombinant RNA polynucleotide of claim 11, wherein the targeting sequence is selected for inclusion in the RNA polynucleotide such that the RNA polynucleotide is suitable for use in CRISPR-based modification of a known DNA target sequence comprising the protospacer.

15. The recombinant RNA polynucleotide of claim 11, wherein the spacer is not more than 29 nucleotides in length.

16. The recombinant RNA polynucleotide of any one of claims 1-11, wherein the first and/or second reference repeat sequence is the same as a repeat sequence present in a bacterium or archaea, wherein the repeat sequence in the bacterium or archaea is contiguous with a spacer in a CRISPR array that is not the most recently acquired spacer acquired by the bacterium.

17. An expression vector encoding the engineered guide polynucleotide of any one of claims 1-11.

18. An isolated RNA polynucleotide transcribed from the expression vector of claim 17.

19. A cell comprising the expression vector of claim 17.

20. A system for modifying a genetic target in one or more cells, the system comprising a first set of transposon genes tnsA, tnsB, tnsC, and tniQ, Cas genes cas8f cas5f cas7f and cas6f and optionally an xre gene encoding a transcription regulator, or optionally one or more proteins encoded by one or more of said genes, and wherein optionally at least two of said proteins are within a fusion protein, and a sequence encoding the recombinant RNA polynucleotide of any one of claims 1-11, and optionally a DNA cargo that can be introduced into DNA in a location that is proximal to the protospacer.

21. The system of claim 20, wherein the tnsA gene comprises a change in sequence such that at least one amino acid in the TnsA protein encoded by the tnsA gene is changed relative to its wild type sequence, or if the protein is used the protein comprises said change.

22. The system of claim 20, wherein:
i) the tnsB gene comprises a change in sequence such that at least one amino acid in the TnsB protein encoded by the tnsB gene is changed relative to its wild type sequence or if the protein is used the protein comprises said change; or
ii) the tnsC gene comprises a change in sequence such that at least one amino acid in the TnsC protein encoded by the tnsC gene is changed relative to its wild type sequence or if the protein is used the protein comprises said change.

23. The system of claim 20, wherein:
a) the change in the TnsA protein comprises a change of Ala at position 125 of an Aeromonas salmonicida TnsA protein, wherein optionally the change is to an Asp, or is a homologous change in a homologous TnsA protein;
b) the change in the TnsB protein comprises a change of amino acid position 167 of an Aeromonas salmonicida TsnB protein, wherein optionally the change is to Ser, or is a homologous change in a homologous position of a homologous TnsB protein; or
c) the change in the TnsC protein comprises a change in at least one amino acid in position 135, 136, 137, 138, 139, or 140 of the Aeromonas salmonicida TnsC protein, wherein optionally the change is to an amino acid at position 140 in the TnsC protein, wherein optionally the change to amino acid 140 is a change to an Ala or Gln, or a is a homologous change in a homologous position of a homologous TnsC protein.

24. A method comprising expressing the recombinant RNA polynucleotide of any one of claims 1-11 in cells comprising first transposon genes tnsA, tnsB, tnsC, and optionally at least one tniQ, Cas genes cas8f cas5f cas7f, and cas6f, and optionally xre, wherein optionally at least one of the first set of transposon genes or the Cas genes are present within a recombinant polynucleotide.

25. The method of claim 24, wherein the targeting sequence is targeted to a protospacer in a chromosome or a plasmid in the cells.

26. The method of claim 24, wherein the cells are prokaryotic cells.

27. The method of claim 24, wherein the cells are eukaryotic cells and the targeting sequence targets the chromosome.

28. The method of claim 24, further comprising introducing a cargo DNA in the cells, wherein the DNA cargo is inserted into the chromosome or plasmid in a position that is proximal to the protospacer.

29. The method of claim 28, wherein the DNA cargo comprises transposon left and right ends.

30. The method of claim 29, wherein the DNA cargo is inserted into the chromosome or the plasmid at a position that is 48 nucleotides from an end of the protospacer.

31. A method comprising analyzing CRISPR arrays from a plurality of organisms, determining repeat sequences flanking spacers in the CRISPR arrays, comparing repeat sequences flanking earlier acquired spacers to repeat sequences flanking later acquired spacers, determining differences between repeat sequences flanking the earlier and later acquired spacers, and designating the repeat sequences flanking the earlier acquired spacers that are different from the repeat sequences flanking the later acquired spacers as candidates for use in designing a guide RNA for use in CRISPR-based DNA modification, wherein optionally the CRISPR-based modification is improved relative to a CRISPR-based DNA modification using a guide RNA that is transcribed from sequences flanking the later acquired spacers.

32. The method of claim 31, further comprising producing an RNA polynucleotide comprising 5′ end and/or 3′ end sequences that are transcribed from the repeat sequences flanking the earlier acquired repeat sequences.

33. The method of claim 32, further comprising using the RNA polynucleotide in a CRISPR-based DNA modification.

34. The method of claim 33, wherein the RNA polynucleotide comprises a substitution of a spacer in analyzed CRISPR arrays with a different spacer sequence that is targeted to a predetermined DNA sequence present in a chromosome or plasmid, and wherein said spacer is optionally not longer than 29 nucleotides in length.

35. An RNA polynucleotide produced according to the method of claim 31.

36. A library of expression vectors encoding RNA polynucleotides identified by the method of claim 31.

37. A database comprising a plurality of entries, the entries comprising or consisting of repeat sequences flanking earlier acquired spacers identified according to the method of claim 31.

38. A method comprising selecting one or more repeat sequences from the database of claim 37, and producing an expression vector encoding the one or more repeat sequences.

39. A kit for producing an expression vector for use in CRISPR-based DNA modification, the kit comprising a vector comprising one or more restriction endonuclease recognition sites configured for cloning a desired spacer such that the spacer is contiguous with one or more repeat sequences identified according to the method of claim 31.

40. The kit of claim 39, further comprising one or more expression vectors encoding a first set of transposon genes tnsA, tnsB, tnsC, and tniQ, Cas genes cas8f, cas5f cas7f, and cas6f and optionally an xre gene, or optionally one or more proteins encoded by one or more of said genes.

41. A method for modifying a DNA target sequence, the method comprising contacting the DNA target sequence with
i) a guide polynucleotide comprising a spacer sequence and a CRISPR repeat sequence, and
ii) a type I-F CRISPR-Cas protein,
wherein the spacer sequence comprises a targeting sequence that is complementary to a protospacer sequence the DNA target sequence, wherein the CRISPR repeat sequence comprises a nucleotide change relative to a reference repeat sequence, and wherein guide polynucleotide directs the type I-F CRISPR-Cas protein to effect a modification in the DNA target sequence.

42. The method of claim 41, wherein the guide polynucleotide further comprises a second CRISPR repeat sequence, wherein the second CRISPR repeat sequence comprises a nucleotide change relative to a second reference repeat sequence.

43. The method of claim 41 or 42, wherein the first reference repeat sequence is encoded by a first occurring repeat sequence that is 3′ to a Cas6 coding sequence in an endogenous prokaryotic CRISPR array, and/or wherein the second reference repeat sequence is encoded by a second occurring repeat sequence that is 3′ to the Cas6 coding sequence in the endogenous prokaryotic CRISPR array, and wherein the endogenous prokaryotic CRISPR array is optionally a gammaproteobacteria CRISPR array.

44. The method of any one of claims 41-43, wherein the CRISPR repeat sequence comprises three contiguous nucleotides at a 5′ end that are not GTG or GUG.

45. The method of any one of claims 41-44, wherein the modification is more efficient as compared to a modification induced by the type I-F CRISPR-Cas protein and a reference guide RNA that comprises the first or second reference repeat sequence and does not comprise the nucleotide change.

46. The method of any one of claims 41-45, wherein the type I-F CRISPR-Cas protein comprises Cas8, Cas5, Cas7, or Cas6.

47. The method of claim 46, comprising contacting the DNA target sequence with Cas8, Cas5, Cas7 and Cas6.

48. The method of claim 47, wherein two or more of the Cas8, Cas5, Cas7 and Cas6 proteins are connected by a linker.

49. The method of any one of claims 41-48, further comprising contacting the DNA target sequence with a transposon protein selected from the group consisting of tnsA, tnsB, tnsC, tniQ, and tnsD.

50. The method of claim 49, wherein the TnsA protein comprises a A125D amino acid substitution as referenced in the TnsA reference sequence.

51. The method of claim 49, wherein the TnsB protein comprises a P167S amino acid substitution as referenced in the TnsB reference sequence.

52. The method of claim 49, wherein the TnsC protein comprises a L135, I136, I137, I138, D139, E140A or E140Q amino acid substation in the TnsC reference sequence.

53. The method of any one of claims 41-52, wherein the modification comprises inserting a DNA cargo into the DNA target sequence.

54. The method of claim 53, wherein the modification does not result in a double stranded break in the DNA target sequence.

55. The method of claim 53 or, wherein the DNA target sequence is in a eukaryotic chromosome.

56. The method of any one of claims 41-55, wherein the DNA target sequence is in a cell.

57. The method of claim 56, wherein the cell is a mammalian cell, optionally wherein the cell is a human cell.

58. The method of any one of claims 52-57, wherein the DNA target sequence is in a subject.

59. The method of claim 58, wherein the subject has a disease, and wherein the DNA cargo comprises a DNA sequence that encodes a protein, wherein expression of the protein in the subject treats or ameliorates the disease.

60. A method for treatment of a disease in a subject in need thereof, the method comprising administering the engineered polynucleotide of any one of claims 1-11, the vector of claim 17, the cell of claim 19, or the system of any one of claims 20-23 to the subject, wherein the modification treats or ameliorates a symptom of the disease in the subject.
